SEQUENCE LISTING (79 sequences)

SEQ ID NO: 1 - protein, 149 aa, synthetic construct
MSTSTSQIAVEYPIPVYRFIVSVGDEKIPFNSVSGLDISYDTIEYRDGVGNWFKMPGQSQSTNITLRKGVFPGKTELFDWINSIQLNQVEKKDITISLTNDAGTELLMTWNVSNAFPTSLTSPSFDATSNDIAVQEITLMADRVIMQAV

SEQ ID NO: 2 - DNA, 450 bp, synthetic construct
  source 1..450: mol_type = other DNA
atgtctacaagtacatctcaaattgcggttgaatatcctattcctgtctatcgctttattgtttctgtcggagatgagaaaattccatttaatagtgtttcaggattagatattagttatgacaccattgaataccgagatggtgttggtaattggttcaaaatgccgggtcagagtcagagcactaatatcaccttgcgtaaaggcgttttcccggggaaaacagaactgtttgattggattaactctattcagcttaatcaggtagagaaaaaggatattaccatcagtttaactaatgatgcaggtaccgaattattaatgacctggaatgtttctaatgcttttcccacttcattgacttcaccttcatttgatgccaccagtaatgatattgcagtacaggaaattacgctgatggcagatcgggtgattatgcaggctgtttga

SEQ ID NO: 3 - protein, 152 aa, synthetic construct
MNDYYTPVVSHRFMASFIFNRIPDPLDIRFQRISGLSRELQVTQYSEGGENARNNYLAEKIQHGTLTLERGVMTVSPLTWMFDRVLSGEKIAYADVVVMLLNENSLPLSSWTLSNALPVRWQTSDFDANSNAILVNTLELRYQDMRWLGVKI

SEQ ID NO: 4 - DNA, 459 bp, synthetic construct
  source 1..459: mol_type = other DNA
atgaacgattattacacacccgtggtatcccatcgttttatggcgagttttatttttaaccgcattcccgatccgctggatattcgttttcagcgtatctctggccttagtcgggaactacaggtgactcagtacagtgagggaggagaaaatgcccgtaataactatttagctgagaaaatccaacacggtacgttgactttggaacggggcgtgatgacagtctcgccattgacctggatgtttgatcgggtattgagtggtgaaaaaatcgcttatgccgatgtggtggtgatgctactgaatgaaaattcactgccattgtccagttggacgttgagcaatgcgctgccggtacgctggcaaaccagcgactttgacgctaacagcaatgccatattggtgaatacccttgaattgcgttaccaggatatgcgctggcttggagtcaaaatatga

SEQ ID NO: 5 - protein, 229 aa, synthetic construct
MSLIERGLAKLTINAYKDREGKIRAGTLQAMYNPDSLQLDYQTDYQQSQAINSEKQSSIYVQAKPAGLSLELIFDATMPGNKTPIEEQLMQLKQLCSVDATSNETRFLQVKWGKMRWESRGYFAGRAKSLSVNYTLFDRDATPLRVRVILALVADESLVLQETEQNLQSPAKIALRIQDGVSLALMAASTASTLSGGVDYLTLAWQNGLDNLNGFVPGEILQATRGDES

SEQ ID NO: 6 - DNA, 690 bp, synthetic construct
  source 1..690: mol_type = other DNA
atgagtctgattgaacgtggtttagctaagctgacaattaatgcttataaggatagggaagggaagatacgggcaggaacgttgcaggccatgtataaccctgactccttgcaactggattaccaaacggattatcagcaatcccaagcgattaatagcgaaaagcaaagtagcatttatgtacaggccaagcccgcagggttatcacttgaattaatttttgatgccacgatgccgggtaacaaaacccccattgaagagcagctcatgcagctcaagcaactgtgcagtgtggatgcaaccagtaacgagacgcgattcctgcaagttaaatggggcaaaatgcgttgggaaagtcggggttactttgctggcagggccaagagtttgtctgtgaattacactttgtttgatcgtgatgcgactcccttgagggtacgggtaatattggcattagtggctgatgaaagtctggtgttgcaggagactgaacaaaatctgcaatctccggcaaaaatcgcattacgcatacaggatggggtatctctggctctgatggcagccagtacggcatcaacattgtcaggcggtgtggattatctgacgctggcctggcaaaacggtctggataatctcaatgggttcgttccgggtgaaatattgcaggccaccaggggagacgaatcatga

SEQ ID NO: 7 - protein, 355 aa, synthetic construct
MTTVTSYPGVYIEELNSLALSVSNSATAVPVFAVDEQNQYISEDNAIRINSWMDYLNLIGNFNNEDKLDVSVRAYFANGGGYCYLVKTTSLEKIIPTLDDVTLLVAAGEDIKTTVDVLCQPGKGLFAVFDGPETELTINGAEEAKQAYTATPFAAVYYPWLKADWANIDIPPSAVMAGVYASVDLSRGVWKAPANVALKGGLEPKFLVTDELQGEYNTGRAINMIRNFSNTGTTVWGARTLEDKDNWRYVPVRRLFNSVERDIKRAMSFAMFEPNNQPTWERVRAAISNYLYSLWQQGGLAGSKEEDAYFVQIGKGITMTQEQIDAGQMIVKVGLAAVRPAEFIILQFTQDVEQR

SEQ ID NO: 8 - DNA, 1068 bp, synthetic construct
  source 1..1068: mol_type = other DNA
atgacaaccgttaccagttatcctggcgtttatattgaagaattaaatagcctggccttgtcagtttcaaatagcgccacagcggttcctgtttttgctgtggacgaacaaaaccaatatattagtgaagataatgcaatccgtattaattcgtggatggattatcttaatctgattggcaattttaataatgaagacaaattagatgtttctgtgcgtgcttattttgccaatggaggtggatattgttatctcgtcaaaacaacgagtttagaaaaaattattccaaccttggatgatgtaaccttattggttgctgcgggcgaagatattaaaacgacagtagatgttttatgtcagccaggaaaagggttattcgcagtctttgatggccctgaaacagagttgactatcaacggtgcggaagaggcaaaacaagcctataccgccacaccattcgctgcggtttattatccttggttgaaagcggattgggctaacatagatattccacccagtgcagtgatggcgggagtttatgcatcggtggatttatcccgtggtgtatggaaagcgcctgccaatgttgcgttgaaagggggcctggaacctaaatttttagtcacggatgaattgcagggtgaatataacactggccgcgctatcaatatgattcgtaatttcagtaacacaggtactacggtttggggtgcaagaaccctggaagataaagacaattggcgttatgttccagtgcgacgcttgtttaattctgtggagcgggatatcaagcgtgccatgagctttgctatgttcgagcctaataatcagcctacttgggagcgggtacgggcggcgattagcaactacctttatagcctgtggcaacaggggggattagctggcagcaaagaagaagacgcttattttgtgcaaattggtaaaggtataacgatgacacaggagcagattgatgcagggcaaatgattgttaaagtcggtttggctgctgtacggcctgcggaatttatcattctccagtttacgcaagatgtagaacagcgttaa

SEQ ID NO: 9 - protein, 440 aa, synthetic construct
MSAILKAPGVYIEEDASLALSVSNSATAVPVFIGKFTPTVVDSIQVCTRISNWLEFTSSFSLAPTVEIVVQSNTESESESETYHYIETINLSPAVEALRLYFQNGGGACYIYPLNDAEDELVLAAIPEVIEQKGDITLLVCPELDLDYKTKIYGAVSSLLNDNKVGYFLIADSNDGESVSGVWNSAKAAAYYPQLETNLKFSTLPGDKDIRISGYQDDDETHKPKNLDELRTINEALAQDIDARLLEEKQRAVIIPPSAAIAGIYCQTDNRRGVWKAPANVALTGIGSLLDKVDDERQGEMNDKGINVIRSFTDRGFMVWGARTCVDAANISWRYIPVRRLFNSVERDIRQALRAVLFETNSQPTWVRAKAAVDQYLYTLWQKNALMGARPEEAYFVQIGQDITMSEADIKQGKMIMTVGLAAVRPAEFIILQFTQDVVQ

SEQ ID NO: 10 - DNA, 1323 bp, synthetic construct
  source 1..1323: mol_type = other DNA
atgtctgctattctgaaagcgcctggcgtttatattgaagaagacgcttccctagcgttgtctgtcagtaacagcgcgactgccgtgcctgtttttatcggaaaatttactccgacagtggttgattcaatccaagtctgtacccgtatcagcaactggcttgaattcacttcctctttttccctagctccaacagttgagattgttgtccaatctaacactgaatctgaatctgaatctgaaacttaccactatattgagacaatcaatttatctccagctgtggaagcattgcgactctattttcaaaatggcggaggagcttgctatatctacccattaaatgatgctgaagatgaattggttctggcggccataccagaagtcattgaacagaaaggtgatattactctgttggtttgcccggaactcgatctggattacaaaactaagatctatggcgcagtgagctcactgttgaatgataacaaagtgggctatttcctgattgcggatagcaatgatggagaatctgtgtcaggagtatggaatagtgctaaggccgccgcctattatccccagttggaaactaacctaaaattttccacgttgcctggggataaggacattcgtatcagcggttatcaggatgatgatgaaacacataaaccgaaaaacttggatgagctcaggacaatcaacgaggcgttggcacaggatattgatgcaagattgctcgaggagaaacaacgtgctgtcatcattccgccaagtgctgccattgcgggcatttattgccaaacggataatcgtcgcggtgtttggaaagcgccagccaacgttgcgctcacagggatcgggagtttgcttgataaggtagacgatgaacggcagggagagatgaatgacaagggaatcaatgtcatccgttcatttaccgaccgtggttttatggtctggggagcccgtacttgtgtggacgctgccaacatcagctggcgttatattcctgttcgtcgcctgttcaattccgttgaacgagatatccgccaggcgctgcgcgctgtgttgtttgaaactaatagtcagcctacctgggtacgtgctaaggctgccgttgatcaatatctttataccctttggcagaaaaatgcattgatgggtgctcgcccggaagaagcttattttgtgcaaattggtcaggatatcaccatgtccgaggctgatattaaacagggtaagatgatcatgactgttggtttggcagcagtgcggccagctgagttcatcattctgcaatttacgcaggatgttgttcagtaa

SEQ ID NO: 11 - protein, 424 aa, synthetic construct
LTVPTLTILEEVMMMERLQPGVTLTESIITMGQQEIPSAVPVFIGYTVRYPEQSEASVRIDSLAEYTSLFGDDHVMMFAVRHYFDNGGQQAFVLPLKDNMPSVEMTTAEAENLIAALRSATVSEAIGGHSQITLILVPDMARLNDSDIDDSSTQVSLWSQGWEALLQLSQVRPNLFVLLDAPDNVEQAQKCMTTLSSDYRQWGAAYWPRLETTYQKEISGKDNESQGIFQGTVLSPTAAVAAVIQRTDNDAGVWKAPANIALSQVIRPVKSYLQGSVLFNSSGTSLNVIRSFPGKGIRVWGCRTLENTDNTQWRYLQTRRLVSYVTAHLTQLARMYVFEPNNELTWMKLKGQSYNWLRQLWLQGGLYGSQEDEAFNILLGVNETMTEDDVRAGKMIMKVELAVLFPAEFIEISLVFNTQTEALS

SEQ ID NO: 12 - DNA, 1275 bp, synthetic construct
  source 1..1275: mol_type = other DNA
ttgacagtgcctactctaaccatcttggaggaggtgatgatgatggagagactccaaccgggtgtgactttaacagaaagtataatcacgatgggtcagcaagagatacccagtgctgtgccggtgtttattggttacaccgttcgttatccggaacaatcggaagcatcagtccgtatcgacagtttggccgagtataccagcctgtttggtgacgaccatgtgatgatgtttgctgtcaggcactattttgataatggcgggcaacaggcatttgttttacccctgaaggacaatatgccatcagtggagatgaccacagctgaagcggaaaatctgatagccgcattgcgctctgctacggttagcgaagccattggtgggcatagtcagattacactgattttggtaccggatatggctcggcttaatgacagtgatattgatgactcctcaacccaggtaagcctgtggtcccaaggctgggaggcgctgctgcaattgagtcaggttaggcccaacctctttgtgctgttagatgcgccggataatgttgaacaggcgcagaagtgtatgacaacgctatcgtcagattatcgtcaatggggggcagcatattggcctcgtctggaaactacctatcagaaagaaatatctggcaaggacaatgaatctcagggaattttccaggggactgttctgtcacccacagccgcggtcgcagcggtaattcaacgcacggataacgacgcgggtgtttggaaagcaccggccaatattgccttatcccaggttattcgacctgttaaatcttatcttcagggaagtgtactgtttaacagcagcggcacttcgctcaatgtgatccgcagtttcccaggtaagggcatacgggtatggggatgccgcactctggaaaacacggataatacgcagtggcgctatctgcaaacacgtcggctggtttcctatgtaacagcgcatttgacccaattggctcgcatgtatgtctttgagccaaataatgaacttacctggatgaagttaaaaggacaaagttacaactggttacggcaattatggttgcagggtggcttgtatggttcacaggaggatgaggcatttaacattctgttaggcgtaaacgagacgatgactgaggatgatgttcgtgcaggaaaaatgatcatgaaagttgagttggctgtgttgtttcctgccgaatttattgagatcagtttggtgtttaatacccaaacagaggcgctgtcttaa

SEQ ID NO: 13 - protein, 728 aa, synthetic construct
MELNELTNKLSNLVPMTDFKLDNRASLQLLKYIEAYTKIIPFNSGDKYWNDFFFMSGNTPEKLAKLYQKEIEPNGELLPQQAFLLAVLRLLETPISLLNVLPAAHRELYYRELLGLSSHAAQPDQVALSMELNSTVMEQLLPEGTLFEAGQDEQGNALQYALDASLLANRGYISDLRWLRNDGEKQWVTSAPWDLQAQVSLPSDGIRLFGKTNSDQQVFGGVLITSSLLAMEAGIRKIIVTFEQEMNTQELVAQVSSGNQWLTLTSEVNKKEVTLTLSDKEPAISAPEDLDNLFFTQPVLRLQGKDSQALPEVTGISVSEKDDTKDTSFEMYHLTPFGYSSDIEPLEENPALYLGFTDVKPGQTLALYWKLKSPQQPTVSWYYLDQHNQWAELDSWVSDGTQNLYQDGTWHVELPVDASNQAEQMPVGRYWLRAVVEVPAHEGALGKAPWLYGLIYNAMTATLVNVDSISDSHFLTPLPASSIQRPVEPIIVLASVNQPWASWGGRIPESYSAFFERIAQNLSHRNRSLTWGNMVTLLKERYVSIFDVKYPGNDELTRVPALEQQQLTVIPANRYNDSDDSLRPVLNPARLQEMADWLQQKDSPWASIEVRNPEYLDVKIHYEVIFKPDVNEDFGYRQLQQQLCEVYMPWSIDEQRPVVLNNSINYFQLLATIQQQPLVERVTRLTLHRADSSDESDGTASVEAKDNEVLILVWEEDDNLQYRGNDYE

SEQ ID NO: 14 - DNA, 2187 bp, synthetic construct
  source 1..2187: mol_type = other DNA
atggaattaaatgagttaactaacaaattgtcaaatttggtgccaatgaccgattttaaattagataatcgagccagtttgcaattgcttaaatatattgaagcgtatacgaagataataccctttaattctggcgataaatattggaatgactttttctttatgtcaggaaatacgccagagaaacttgcaaaattatatcagaaagaaatagaacccaatggggagttattacctcagcaggcttttttgttggcggttttgcgtttattggaaacaccaatatccttattaaatgtattacctgctgctcatcgtgagctctattatcgggagcttttaggcttgtcttcccatgcggcacagcctgatcaggttgctttatctatggaactgaattcgacagtgatggaacagctgctccctgaaggaaccctgtttgaggctggtcaggatgaacaaggcaatgcattgcaatatgccctggatgccagtttgctggctaatcgtggatatatcagtgacttgcgctggttacggaatgacggggaaaagcaatgggttacttctgctccatgggatttacaggcacaggtgtcactgccgtctgatgggatacgattatttggtaagacaaatagtgatcagcaggtatttggtggggtgttgataacgtcatcacttctggcgatggaagcggggataaggaagatcattgttacttttgagcaggagatgaacacccaagaactggtggcacaggtcagcagtggaaatcaatggctaacattgacgtctgaggtaaataagaaagaggtcacactgacactgtcagacaaagaaccggcaatcagtgcgccagaggatctggataatctctttttcacgcaaccggtactcaggctacagggaaaggatagtcaggcactgccggaggtgacgggtatcagcgtttcggaaaaggatgatactaaggatacctcttttgagatgtatcacttaacaccatttggttatagcagtgatatagagccattggaggaaaatccagcgttatatttaggctttactgatgtaaagccagggcaaacactggcgctgtattggaaattaaaatccccgcagcaaccaaccgtttcctggtattacctggatcaacataatcaatgggctgaattggattcatgggtcagtgatggaacccagaatctgtatcaggatggtacttggcacgttgagttgcctgtggatgcatccaatcaggcagagcagatgccagttggacgctattggttgcgggcagtggtggaggtacccgctcatgagggggcgttggggaaggctccttggctatatggtctaatctataacgccatgacggcaaccttggttaatgtagatagcatcagtgacagccatttcttaacccctttgcctgccagcagcatacagcggcccgttgaacccatcattgtgttggcatcggtcaaccagccttgggcatcatggggtggacgtatacctgaatcctacagtgccttttttgaacggatagctcaaaacctgtctcatcgaaaccggtccttaacctggggaaatatggtgacattactcaaagagcgttatgtcagcatctttgatgttaagtatccaggtaatgatgaactcaccagagtgccagcattggagcagcagcaactaacagtgattccagcaaaccggtacaacgatagcgatgattctctgcgtccggtactgaatcctgctcgtctgcaagagatggctgattggttgcagcagaaagactctccctgggcctctattgaggtcaggaatccagaatacttggatgtgaaaatccattacgaggtgatttttaaacctgatgtgaacgaagattttggctatcgccagctacagcagcaactgtgtgaggtgtatatgccttggagcatagatgagcagcggcccgttgtattgaataacagcattaattatttccagttgttagccactattcaacagcaaccgctggttgagcgagtcactcgtctgacactacatcgggctgattcttctgatgagagtgatggtacagcatctgtggaagccaaagataatgaagtgcttattttagtctgggaagaggacgataatctgcaataccgaggaaatgactatgagtaa

SEQ ID NO: 15 - protein, 950 aa, synthetic construct
MSNQDALFHSVKDDIHFDTLLEQAHQVIEKQAEKLWSDTAEHDPGITFLQGISYGVSDLAYRHTLPLKDLLTPAPDEQQQEGIFPAEFGPHNTLTCGPVTADDYRKALLDLHSSDSLDGTQQDEGDFLFRSVQLVREPEKQRYTYWYDATKREYSFVNSEGAKEFTLRGNYWLYLEPTRWTQGNIAAATRQLTEFLTKNRNIGESVSNIIWLQPVDLPLLLDVELDDDVGAQDVPGIFAAVYSTAEQYLMPGAQRYRTEVLQNAGMSNDQIFEGPLLEHGWIPELPAARDYTQRLTLNLSRLVNSLLEIEGIKHVNRLRLDDSFDKTAIEPVKGDTWSWSIKEGYYPRLWGEDPLNQLAQQNGPLRVIAKGGISVSVSKEQIQASLPSQSLIQNEPVILAYGQHRDVGSYYPVSDTLPPCYGLQHSLSESEHLLPLHQFMLPFEQLLACGCQQIAMLPRLLAFQREGYEVWGDQWPFKSGSVNDDAHQDYAPALKDLLGQIALDSDHELDIINYLLGYFGTQRAPRTFTTQLDDFRAVQQGYLAQQPTLTYHRSNIRIDQVSSLQKRIAARMGLGGELFKPQPDLSQLPFYLIEHRALLPVKPNSQFDKEQKPASVTEEGGSQTGQHYVVIEQKGIDGKLTQGQVINLILYEGEQGETQFTIRGQMVFKTEGDKFWLDVNNSAQLEYNLARVMTAAKASKLFWQNSPVWMEDMGYRLAYASDQSSLPVNQRRLTRTVQTPFPPMVVVGSEITLLKQVGIVNLKKAESEKLYAKVVSFDRIEGTLIIERLGNSTLAFPTSEEAWRYSWYFSGEKYERTDRFSFVISVVVNSDLIKLPGVDPYKLEEWVKETILTEFPAHISMIIHWMDREAFLNFANTYQRWQNNGTPLGDAAYSILESLTLGKLPSALKGVGTMRIATSSQREEVVGSNGDQWNTDGITQNELFYVPKES

SEQ ID NO: 16 - DNA, 2853 bp, synthetic construct
  source 1..2853: mol_type = other DNA
atgagtaatcaggatgcactgtttcatagcgttaaagacgatattcactttgataccttgctggaacaagctcatcaggtgattgaaaaacaggctgaaaaactgtggagtgatacggcagagcatgatccgggtatcacatttttgcagggaatcagttacggtgtgtcagatttggcttaccgacatacattacccctgaaagatttactgactccggcgccggatgagcagcagcaagagggaatttttcctgccgaatttggcccgcataatacactgacttgtgggccggtgacagcggatgattatcgcaaggcattgttagatctacacagcagcgacagcctggatggtactcagcaggatgagggggattttctgttccggagtgtgcaactggtgcgtgaaccggaaaaacagcgttatacctattggtatgatgcaaccaagagggaatatagctttgtcaacagtgaaggggctaaagagtttaccttgcgggggaattactggttgtatctggaaccaacccgttggactcagggtaatattgccgctgctaccagacaactgacagaatttttgactaaaaatcgcaatattggtgaatctgtcagcaacattatctggctacaaccggttgatctgccactgttgctggatgttgaactggatgatgatgtaggtgcacaggatgtccccggtatttttgcggcggtgtatagcaccgcagagcagtatctgatgcctggagcacagcgttaccgtacggaagtactgcaaaatgctgggatgagcaatgatcaaatcttcgaaggtccattattggaacatggctggataccagagctgccggcagcccgtgattatactcaaaggctcactctcaatcttagccggttggtaaatagtctgcttgagattgagggcattaaacatgtgaatcgtcttcgtctggatgatagcttcgataaaactgctattgaacccgttaagggggatacctggtcgtggtcgatcaaagagggctattatccacgtctttggggagaagacccacttaaccaattggcgcaacaaaatggcccgcttagggtgatagccaaaggagggattagcgtcagtgtgagtaaagagcaaatccaggccagtttacccagtcaatcactgattcaaaatgagccggtaatattggcttacggccagcaccgtgacgttggcagctattatcccgtcagtgatactttgccgccttgctatggactacaacattctttgtctgaaagtgaacacttattgccacttcatcaatttatgttgccatttgaacaattattggcctgtggttgtcaacagatagccatgctcccgcggttactggcttttcagcgcgaaggttatgaggtttggggtgatcagtggccctttaagtcaggctcagtgaatgatgacgcccatcaagattatgcccctgcattaaaggatttgttaggacagattgcgctggatagtgatcatgaattggatattattaattacttgctgggttactttggcacacagcgggcaccgcgtacctttacgacacaactcgatgattttcgtgcggtccaacagggttatctggcccagcaaccgacattgacttaccaccgctccaatattcgtatcgatcaggtatcgtcgctacaaaaacgtattgctgctcgcatggggctgggcggtgagttgtttaaacctcaaccggatctgagccaactgcctttttatttgattgaacatcgagcgttgctgccagtcaaacccaatagtcagtttgataaggaacagaaaccagcctcggtgacagaggaggggggcagccaaacaggtcaacattatgtggtcattgaacagaagggcattgatggcaagctgacacaggggcaagtgatcaatttaattctgtatgaaggagagcagggagaaacccaatttacgatacgcggtcagatggtattcaaaaccgagggggataagttttggttggatgtgaataatagtgcgcaactggaatataatctggcgcgggtaatgacagcagccaaggcgagtaaactcttttggcaaaacagcccggtatggatggaggatatgggctatcgtctggcctatgctagtgaccaatcctcattgcctgtgaatcaacggcgcttgacccgcacagtgcaaactccattcccgccgatggttgttgtaggtagcgaaatcaccctgttaaagcaggtggggatagtcaatttaaaaaaagcggagtcagaaaaactttatgcaaaagttgttagctttgatcgcattgaagggaccttgattattgagcgtttgggtaattccactctggcttttcctacctcggaagaggcgtggcggtatagttggtatttttcgggggagaaatatgaaaggactgaccgcttttcatttgtgattagcgtagtagtgaacagtgacttaattaaattgcccggtgttgatccctataaattggaagaatgggtgaaagaaacgattcttaccgaatttccagctcatatttctatgattatccattggatggatcgggaagcctttttaaatttcgccaatacctatcagcgttggcaaaataatggtacgccactgggggatgcggcttattccattctagaaagtttgacacttggtaaattgccatctgccttaaaaggtgttggcacaatgcgtattgccacatctagtcaaagagaagaagtggtgggtagtaatggtgatcaatggaatacagatggaataacccagaatgaattattctatgttcctaaagagagctag

SEQ ID NO: 17 - protein, 508 aa, synthetic construct
MNETRYNATVQEQQTLSNPKAVGPDIDKLKDKFKEGSIPLQTDFNELIDIADIGRKACGQAPQQNGPGEGLKLADDGTLNLKIGTFSNKDFSPLILKDDVLSVDLGSGLTNETNGICVGQGDGITVNTSNVAVKQGNGISVTSSGGVAVKVSANKGLSVDSSGVAVKVNTDKGISVDGNGVAVKVNTSKGISVDNTGVAVIANASKGISVDGSGVAVIANTSKGISVDGSGVAVIANTSKGISVDNTGVAVIANASKGISVDGSGVAVIANTSKGISVDGSGVAVIANTSKGISVDSSGVAVKVKANGGIKVDANGVAIDPNNVLPKGVIVMFSGSTAPTGWALCDGNNGTPNLIDRFILGGKGTDINGVSTNTASGTKNSKLFDFSSDEATLTIDGKTLGRALSLQQIPNHAHFSGIIMDTEKVNYYGSKKITTNVWGVTTGDNTSVRYIYKSSGVLDSNNNVSNSTLGGNSLQTHDHDIKITGTGKHSHKNKVTVPYYILAFIIKL

SEQ ID NO: 18 - DNA, 1527 bp, synthetic construct
  source 1..1527: mol_type = other DNA
atgaacgaaactcgttataatgcaactgtacaagaacaacaaacattatctaatccaaaagctgttggacctgacatcgataaattaaaggataaatttaaagagggcagtattcccctgcaaaccgatttcaatgagttaattgatattgccgatattggacgtaaagcctgtggtcaagcgccacaacaaaatggcccaggagaaggattgaaattggctgatgacggtacgcttaatttaaaaataggcactttttccaataaagacttttctccattaatattaaaagatgatgttttatctgtagatcttggtagtggtctgactaatgaaaccaatggaatctgtgtcggtcagggcgatggtattacagttaacactagcaatgtagctgtaaaacaaggtaacggaattagcgttactagtagtggtggtgttgccgttaaagttagtgctaataagggacttagcgttgatagtagtggtgttgcagttaaagttaatactgataagggaattagcgttgatggtaatggtgttgcagttaaagttaatactagtaaaggaattagcgttgataatacaggtgttgcagttatagctaatgctagtaagggaattagcgttgatggtagtggtgttgcagttatagctaatactagtaaaggaattagcgttgatggtagtggtgttgcagttatagctaatactagtaaaggaattagcgttgataatacaggtgttgcagttatagctaatgctagtaagggaattagcgttgatggtagtggtgttgcagttatagctaatactagtaaaggaattagcgttgatggtagtggtgttgcagttatagctaatactagtaaaggaattagcgttgatagtagtggtgttgcagttaaagttaaagctaatggcggaattaaagtagatgctaatggtgttgcaattgatcctaataatgtactccccaagggagtgattgtaatgttctctggcagtactgcaccaactggttgggcgttatgtgatggcaataatggtacaccaaatttaatcgatcgatttattttaggtgggaaagggactgatattaatggagtgagtactaatacagcttcaggtactaaaaatagtaagttattcgatttcagttctgatgaagctacattaactattgatggtaaaacactggggagagcattatcgttacagcaaatacctaatcatgcacactttagtggaataattatggatacagagaaagttaattattatggaagtaaaaaaatcacaacaaatgtgtggggtgtaacaacaggagataatacttcagtacgatatatttataagtcatcaggtgtacttgactctaacaataatgtctccaacagtaccttaggcggaaacagtctgcagacgcacgatcatgatattaagataacgggcacaggaaaacattctcacaaaaacaaagtaacagtcccttattatattctggctttcatcataaagctttaa

SEQ ID NO: 19 - protein, 538 aa, synthetic construct
MSHQLKIIADGKALSLLAAVDVDTCYRVNSIPSATLKLSVPDRPLSSFSQTDVQTELAHCQVGKTLRLELIDGSKKWVLFNGLITRKALRIKNKQLLLTLVVKHRLQLMVDTQHSQLFKDKSEKAILSTLLNQTGINARFGKIAALDQKHEQMVQFRCSDWHFLLCRLSATGAWLLPAIEDVQFVQPDALKSNSAYTLKSRGDENKDIVVKDAYWQFDNQINPALLEVSGWDISKQQVQSGGRYGKIALGKAALSPDGLASLNKTGWDICYSSPLTTQESGYLAQGLLLNQRISGVTGEFLLKGDGRYQLGDNIQLTGFGSQLDGTASITEVRHRLNRRIDWETTVSIGLQHEYLPILPDAPELHIATVAKYQQDSAVLNRIPIILPVLNRPNEFLWARLGKPYASHESGFCFYPEPGDEVIIGFFENDPRYPVILGAMHNPKNKAPFEPTQDNREKVLIVKKGEAQQQLVIDGKEKMIRINAGENQIMLQQDKDISLSTKKELTLKAQTMNATMDKSLAMSGKNSVEIKGAKINLTQ

SEQ ID NO: 20 - DNA, 1617 bp, synthetic construct
  source 1..1617: mol_type = other DNA
atgagccaccaactgaaaattattgcagatggtaaggcactgtcacttttggccgcggtagatgtggacacctgttatcgggttaacagtataccttctgcgacattgaaactgagcgtaccggataggccactctcttctttcagtcagacggatgttcagacagaactggcccactgtcaggtagggaaaaccctgcgtctggaattgattgatggtagcaaaaaatgggtgctgtttaatggtcttattacccgtaaggctctgagaattaagaataagcaattattgctcactctggttgtcaagcatcggttgcaactgatggtggatacccagcattcacagctgtttaaagacaaaagcgaaaaagcgatcttaagcacgctattgaatcagaccggaatcaatgctcgcttcggaaagatagcggcgttagatcaaaagcatgaacagatggtgcaatttcgttgttcagactggcattttctgttgtgccgactgtcggcaaccggtgcatggttgttacctgccatagaagacgttcagtttgttcaacctgatgctctgaaatcaaactcagcctataccttgaagagcaggggggatgagaacaaagacatcgttgtcaaggatgcttactggcagtttgacaatcaaatcaaccccgctttgctggaagtcagtggctgggatatcagtaagcagcaggtacaatcaggcggtcgctacggaaaaatcgcgttgggtaaggcggcactctctcctgatggattggcatcccttaataaaacgggttgggacatttgttatagcagtccgttaacaacccaggaaagcggttatctggcacagggattattgcttaaccagcgcatttctggggtgacaggagaatttttgctcaaaggagatgggcgttaccagttgggagacaacattcagctgactggatttggttcacagttagatggtacggcaagcattactgaggttcgccaccgtcttaatcggcgaattgattgggaaaccacggtgagcattggtttacaacatgaatatttgccgatattacctgatgctcccgaactacatattgcgacagtagcgaaatatcagcaggacagtgcggtgttaaaccgtatccccattattctgccggtactgaatcgtcccaatgaatttttgtgggccagattggggaaaccttatgctagccatgaaagcggtttctgtttttacccagagccaggtgacgaagttattattggtttttttgaaaatgatccgcgttatccagttattttaggtgctatgcataatccgaaaaataaggccccttttgaaccaacccaagataatagggaaaaagtattgatcgttaaaaaaggtgaagcgcaacaacaattagtcattgatggcaaagagaaaatgatccgaattaatgcgggtgaaaatcaaataatgcttcagcaagataaagacatttctctgtcaacgaaaaaagaattaacactgaaagcgcagacaatgaatgccacgatggataaatcattggcaatgtccgggaaaaacagtgttgaaatcaaaggcgcaaaaattaatcttacccaatga

SEQ ID NO: 21 - protein, 141 aa, synthetic construct
MSEAIVVDGDVLQFDPNFGNRQVTVPSPGKISGTGHAQVSGKKVCILGDEKQVRVSATYITTTHTTPGTGTITISALDAGQQALQCTSGAALIIKGQQFTAMFTPELPAMNNTVTPPQPDVTTPSSGKGRFITQQNFATVN

SEQ ID NO: 22 - DNA, 426 bp, synthetic construct
  source 1..426: mol_type = other DNA
atgagtgaagcgattgtggtggatggtgacgtgttacagtttgatcccaactttggcaatcggcaggtgacggttcccagcccaggaaaaattagcggcacaggacatgcgcaggtaagtggaaaaaaagtgtgtattctgggggatgagaaacaggtcagggtttctgcaacctatattacaacaacacatactacgccgggaacaggaaccattactatcagtgctctggatgctggccagcaggcccttcagtgtaccagtggggcggctttaattatcaaggggcagcaatttacggcgatgtttacgcctgaattgccagccatgaataatacagtgactccgccacaaccggatgttacgacaccttcatcaggaaaaggacgttttatcactcaacaaaattttgctaccgtaaattag

SEQ ID NO: 23 - protein, 293 aa, synthetic construct
MLNTQTIIDVNKAMDAMLRAYLNQDIAIRFDLPELDTMQSDAMVSIFLYDIHEDLQLRSAESRGFDVYAGRLLPGWVNIKCNYLITYWEASKPATDASSPDSQPDNQAIQVMSQVLNALINNRQLAGIPGAYTQVVPPKESLNSLGNFWQSLGNRPRLSLNYSVTVPVSLNDGQDSATPVTAVSSTVEQTASLSQEVVSHALRELLITELGGGEDNRLVLSKVELSAVKETMTQDSPAQMIILLSVSGITRQEYLKEIDNIFDRWVNNAEVITTIDDCGIRIESITKDNLVGI

SEQ ID NO: 24 - DNA, 882 bp, synthetic construct
  source 1..882: mol_type = other DNA
atgttaaacacgcaaactattattgatgtcaataaggcaatggatgccatgctgcgcgcatatctgaatcaagatattgccattcgttttgatctacctgaattggatactatgcaatctgatgcgatggtaagtatctttctttatgacattcatgaagatttacagcttcgctcggcagaatcaagagggtttgatgtttatgccgggaggttattgcctggttgggtaaatattaaatgtaactatctgattacctattgggaagcttctaagccagcgactgatgccagcagtccggatagccaacctgataaccaggcaatacaagtgatgtcacaagtattaaatgccttgattaataatcgtcaattggcaggtattcctggtgcttatactcaggttgtaccgcctaaagagagtttaaatagcctggggaatttctggcaatcactgggtaatcgcccacggctttctctcaattattcagtgacagtacctgttagcctaaacgatggtcaggatagcgcgactccggttaccgcggtttcttctacagtggaacaaacggcatcgctcagtcaagaagtggttagtcatgctttacgcgaattactcattacggaattaggaggaggagaggataaccggttggtactgagtaaagttgaattatccgcagtgaaagagacgatgactcaagacagtccggctcagatgattatattgttgtctgtttcaggcattacacgacaggaatatttgaaggaaattgataatatctttgatcgttgggtaaataatgctgaagttattaccactattgatgattgtgggattagaattgaaagtataacgaaagataatcttgtaggaatttaa

SEQ ID NO: 25 - protein, 67 aa, synthetic construct
MPRYANYQINPKQNIKNSHGKSSSSDFSSGYLSFSNNSLDDPFIRQQVKREFIWEGHMKEIEEASRL

SEQ ID NO: 26 - DNA, 201 bp, synthetic construct
  source 1..201: mol_type = other DNA
atgcctagatatgctaattatcagataaaccccaaacagaatattaaaaattcacatgggaaatcttcatcgtcagatttttctagtgggtacctttcattctcaaataattcgcttgatgacccttttattcggcagcaagttaagagagaatttatttgggagggacatatgaaggagattgaagaagcttcaagatta

SEQ ID NO: 27 - protein, 60 aa, synthetic construct
MLKYANPQTVATQRTKNTAKKPPSSTSFDGHLELSNGENQPYEGHKIRKIKGLRQHLADR

SEQ ID NO: 28 - DNA, 180 bp, synthetic construct
  source 1..180: mol_type = other DNA
atgttaaaatatgctaatcctcagaccgtagccacacaacgtactaaaaatactgcgaagaaaccgccatcatcaacctcttttgatgggcaccttgaactttcaaatggtgaaaaccagccttacgaaggccataagattaggaaaatcaaaggattgagacaacatcttgcggatagg

SEQ ID NO: 29 - protein, 15 aa, synthetic construct
GGSGGGGSGGGGSGG

SEQ ID NO: 30 - DNA, 45 bp, synthetic construct
  source 1..45: mol_type = other DNA
ggtggctctggtggtggcggttctggcggcggtggttctggcggt

SEQ ID NO: 31 - protein, 698 aa, synthetic construct
MNISPVFYDSLNQDNDRDLSFLFSELERIDLALQHHFYCVESQRSELLDEFLLTEAEVVTRLDKPLGKPHWINDDYLAISQKGNVSLMAASRLMDLIERFELTDFERDVLLLGLLPHFDSRYYRLFSLIQGGQQGRLPSFALALELFCHSALEKQVQQASFLHRAPLMGCQLLSIDTSQKTLAWLQTPFITDSGVYHFLLGHHYIMPALEHCAEWLTPTGIGCYPEGLKQVLGNVLLSDNDNIRPIVLLRGMAGSARAYTITNMMASEGKQTLLVDISKLADSDEKNIILQIKHILRETRMHGACLLLRNFCLLVEQNKQLLDSLSELLNQPELRIVCLIEPYSPLVWLKKIPVLLIEMPLLTPAEKARLLIASLPDNCSEDIDTITLSQRYTFNPETLPLILQEAQLYQQQRDPLDILQQCDIRQALNLRAQQNFGQLAQRIIPKRSLKDLLVSDEIAQQLREILIAIKYREQVLAGGFKDKIAYGTGISALFYGDSGTGKTMAAEVIADHIGVDLIKVDLSTVVNKYIGETEKNLSRIFDLAEQDAGVLFFDEADALFGKRSETKDSQDRHANIEVSYLLQRLENYPGLVILSTNNRGHLDSAFNRRFTFITRFTYPDEKIRKKMWQEIWPRNIKISEDIDFNELAQRTSVTGANIRNIALLSSFFASEQGNDEVSNENIEIALKRELAKVGRLTF

SEQ ID NO: 32 - DNA, 2097 bp, synthetic construct
  source 1..2097: mol_type = other DNA
atgaatatatcgcctgttttttatgattcattgaatcaggataacgaccgtgatctatcgtttttatttagcgaactggaacgaatagatctcgctcttcaacaccatttttattgtgtagaaagtcagcgaagtgagctcctggatgagtttctgctcactgaggcggaagtggtgaccaggctggataagccacttggtaaacctcattggataaatgatgattatctggcgatatcgcaaaagggcaatgtaagcctaatggcagcgtccagattaatggatctgatcgaacgctttgaactgactgattttgagcgcgatgttttactattaggcttattgccccattttgatagccgctattatcgactgttttcgctgattcaagggggacaacagggtcgattaccttcttttgcgctggcattggaactgttttgccactcggcgctggagaaacaggtacagcaagcgagttttctgcaccgggcacctttgatgggttgccagctattatccatcgatactagtcaaaaaacgctggcctggctccagactccctttattactgacagcggggtatatcactttttactggggcatcactacattatgccggctttagaacattgtgctgagtggttaacaccgacagggattggctgttatcctgaaggattaaaacaagtactgggtaacgtattgttatctgacaacgataatattagaccgattgtcttattacggggaatggccggcagtgccagagcttataccattactaatatgatggcttcagaagggaagcaaacactgctggtagatatatccaaacttgctgatagcgatgaaaaaaacattattcttcagataaagcatattttgcgggaaacccgcatgcatggagcatgtttattattacggaatttttgcttgttagtggaacagaataaacaactattggactccctgtcagagttattgaatcaacctgaattaagaattgtttgcctgattgagccttattccccattggtatggctgaaaaagataccggtattactgattgagatgccacttttaacgcctgcggaaaaagccagattgttaattgccagcttaccggataattgttccgaggatattgatacgataactttaagccagcgttacacttttaacccagaaaccctgccattgattttgcaagaggcccagctttatcaacagcagcgagatccgctggatatcttgcagcaatgcgatatacgccaggcattaaatttgcgtgctcaacaaaatttcggtcaattggcacagcggattattcctaagcgctcattaaaggatttattggtatccgatgagattgctcagcagttacgggaaatactcatagcaattaagtatcgggaacaggttctggcgggagggtttaaagataaaattgcctatggcactggtatcagcgccctgttttatggtgattcaggcactggaaaaaccatggcagcagaagtgattgctgaccacattggcgttgacttaataaaagtggatttatctacagtagtgaataaatacatcggtgaaacagaaaaaaacttatcccgtattttcgatttggcggaacaggatgcaggggtattattctttgatgaagctgacgcactgtttggtaaacgcagtgaaactaaagattcccaggacagacatgccaatattgaagtttcttacttattacagcgcctggagaattacccgggtctggtcattttatccaccaataatcgtggtcatttagacagtgcttttaatcgtcgttttactttcattacccgttttacttacccggatgaaaaaatccgtaaaaaaatgtggcaggaaatttggcctagaaatataaaaatatcggaagatatcgattttaacgaattagctcaacgaacaagcgtgactggcgcgaatatccgcaatattgctttattgtcttcattctttgcttcagagcaggggaatgatgaagtcagtaatgaaaatattgaaattgcattgaagcgtgaattagctaaagtcggacgattaacattttaa

SEQ ID NO: 33 - protein, 7 aa, synthetic construct
PKKKRKV

SEQ ID NO: 34 - protein, 10 aa, synthetic construct
PKKKRKVEAS

SEQ ID NO: 35 - protein, 16 aa, synthetic construct
KRPAATKKAGQAKKKK

SEQ ID NO: 36 - protein, 9 aa, synthetic construct
PAAKRVKLD

SEQ ID NO: 37 - protein, 11 aa, synthetic construct
RQRRNELKRSP

SEQ ID NO: 38 - protein, 38 aa, synthetic construct
NQSSNFGPMKGGNFGGRSSGPYGGGGQYFAKPRNQGGY

SEQ ID NO: 39 - protein, 42 aa, synthetic construct
RMRIZFKNKGKDTAELRRRRVEVSVELRKAKKDEQILKRRNV

SEQ ID NO: 40 - protein, 8 aa, synthetic construct
VSRKRPRP

SEQ ID NO: 41 - protein, 8 aa, synthetic construct
PQPKKKPL

SEQ ID NO: 42 - protein, 12 aa, synthetic construct
SALIKKKKKMAP

SEQ ID NO: 43 - protein, 5 aa, synthetic construct
DRLRR

SEQ ID NO: 44 - protein, 7 aa, synthetic construct
PKQKKRK

SEQ ID NO: 45 - protein, 10 aa, synthetic construct
RKLKKKIKKL

SEQ ID NO: 46 - protein, 10 aa, synthetic construct
REKKKFLKRR

SEQ ID NO: 47 - protein, 20 aa, synthetic construct
KRKGDEVDGVDEVAKKKSKK

SEQ ID NO: 48 - protein, 17 aa, synthetic construct
RKCLQAGMNLEARKTKK

SEQ ID NO: 49 - protein, 401 aa, synthetic construct
MSTDATLIRTTPSHAEADATDTLVATPLMPPRRVISPWPGPGEGQSLMRIPVVDIRGMALMPCTPAKARHLLKSGNARPKRNKLGLFYVQLSYEQEPDNQSLVAGVDPGSKFEGLSVVGTKDTVLNLMVEAPDHVKGAVQTRRTMRRARRQRKWRRPKRFHNRLNRMQRIPPSTRSRWEAKARIVAHLRTILPFTDVVVEDVQAVTRKGKGGTWNGSFSPVQVGKEHLYRLLRAMGLTLHLREGWQTKELREQHGLKKTKSKSKQSFESHAVDSWVLAASISGAEHPTCTRLWYMVPAILHRRQLHRLQASKGGVRKPYGGTRSLGVKRGTLVEHKKYGRCTVGGVDRKRNTISLHEYRTNTRLTQAAKVETCRVLTWLSWRSWLLRGKRTSSKGKGSHSS

SEQ ID NO: 50 - protein, 432 aa, synthetic construct
MQPAKQQNWVFQINGDKQPLDMINPGRCRELQNRGKLASFRRFPYVVIQQQTIENPQTKEYILKIDPGSQWTGFAIQCGNDILFRAELNHRGEAIKFDLVKRAWFRRGRRSRNLRYRKKRLNRAKPEGWLAPSIRHRVLTVETWIKRFMRYCPIAWIEIEQVRFDTQKLANPEIDGVEYQQGELQGYEVREYLLQKWGRKCAYCGTENVPLEVEHIQSKSKGGSSRIGNLTLACHVCNVKKGNLDVRDFLAKSPDILNQVLENSTKPLKDAAAVNSTRYAIVKMAKSICENVKCSSGARTKMNRVRQGLEKTHSLDAACVGESGASIRVLTDRPLLITCKGHGSRQSIRVNASGFPAVKNAKTVFTHIAAGDVVRFTIGKDRKKAQAGTYTARVKTPTPKGFEVLIDGARISLSTMSNVVFVHRSDGYGYEL

SEQ ID NO: 51 - protein, 461 aa, synthetic construct
MAVFVIDKHKRPLMPCSEKRARLLLERGRAVVHRQVPFVIRLKDRTVQHSAVQPLRVALDPGSRATGMALVREKNTVDTGTGEVYRERIALNLFELVHRGHRIREQLDQRRNFRRRRRGANLRYRAPRFDNRRRPPGWLAPSLQHRVDTTMAWVRRLCRWAPASAIGIETVRFDTQRLQNPEISGVEYQQGALAGCEVREYLLEKWGRKCAYCGAENVPLEIEHIVPKSRGGSDRVSNLALACRACNQAKGNRDVRAFLADQPERLARILAQAKAPLKDAAAVNATRWALYRALVDTGLPVEAGTGGRTKWNRTRLGLPKTHALDALCVGQVDQVRHWRVPVLGIRCAGRGSYRRTRLTRHGFPRGYLTRNKSAFGFQTGDLIRAVVTKGKKAGTYLGRIAIRASGSFNIQTPMGVVQGIHHRFCTLLQRADGYGYFVQPKPTEAALSSPRLKAGVSSAGN

SEQ ID NO: 52 - protein, 439 aa, synthetic construct
MTTNVVFVIDTNQKPLQPCSAAVARKLLLRGKAAMFRRYPAVIILKKEVDSVGKPKIELRIDPGSKYTGFALVDSKDNADFIIWGTELEHRGAAICKELTKRSAIRRSRRNRKTRYRKKRFERRKPEGWLAPSLQHRVDTTLTWVKRICKFVPIMSISVEQVKFDLQKLENSDIQGIEYQQGTLAGYTLREALLEHWGRKCAYCDVENVFLEIEHIYPKSKGGSDKFSNLTLACHKCNINKGNKSIDEFLLSDHKRLEQIKLHQKKTLKDAAAVNATRKKLVTTLQEKTFLNVLVSDGASTKMTRLSSSLAKRHWIDAGCVNTTLIVILKTLQPLQVKCNGHGNKQFVTMDAYGFPRKSYEPKKVRKDWKAGDIIRVTKKDGTMLMGRVKKAAKKLVYIPFGGKEASFSSENAKAIHRSDGYRYSFAAIDSELLQKMAT

SEQ ID NO: 53 - protein, 419 aa, synthetic construct
MPNKYAFVLDSKGKLLDPTKSKKAWYLIRKGKASLVEEYPLIIKLKREVPKDQVNSDKLILGIDDGTKKVGFALVQKCQTKNKVLFKAVMEQRQDVSKKMEERRGYRRYRRSHKRYRPARFDNRSSSKRKGRIPPSILQKKQAILRVVNKLKKYIRIDKIVLEDVSIDIRKLTEGRELYNWEYQESNRLDENLRKATLYRDDCTCQLCGTTETMLHAHHIMPRRDGGADSIYNLITLCKACHKDKVDNNEYQYKDQFLAIIDSKELSDLKSASHVMQGKTWLRDKLSKIAQLEITSGGNTANKRIDYEIEKSHSNDAICTTGLLPVDNIDDIKEYYIKPLRKKSKAKIKELKCFRQRDLVKYTKRNGETYTGYITSLRIKNNKYNSKVCNFSTLKGKIFRGYGFRNLTLLNRPKGLMIV

SEQ ID NO: 54 - protein, 1409 aa, synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 55 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 56 - protein, 1365 aa, synthetic construct
KYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 57 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 58 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 59 - protein, 5 aa, synthetic construct
VITYF

SEQ ID NO: 60 - protein, 5 aa, synthetic construct
VVVVV

SEQ ID NO: 61 - protein, 7 aa, synthetic construct
KPWWPRR

SEQ ID NO: 62 - protein, 9 aa, synthetic construct
NIVNVSLVK

SEQ ID NO: 63 - protein, 5 aa, synthetic construct
LPFFD

SEQ ID NO: 64 - protein, 4 aa, synthetic construct
KLVF

SEQ ID NO: 65 - protein, 7 aa, synthetic construct
LVEALYL

SEQ ID NO: 66 - protein, 6 aa, synthetic construct
RGFFYT

SEQ ID NO: 67 - protein, 8 aa, synthetic construct
PPKKARED

SEQ ID NO: 68 - DNA, 183 bp, Photorhabdus asymbiotica
  source 1..183: mol_type = genomic DNA
atgacagtagaaatcagagagttacttatccaggcaaaggtagtgccatcaacacgaccgactgaatcagaacggcaaaaccattctttgatacaggaaagtctggatgaggcgacttgggtggaaacgataaaacgcgaagtgttggccgcattacgcgatgaggaagggtggcgtccatga

SEQ ID NO: 69 - protein, 60 aa, Photorhabdus asymbiotica
MTVEIRELLIQAKVVPSTRPTESERQNHSLIQESLDEATWVETIKREVLAALRDEEGWRP

SEQ ID NO: 70 - DNA, 423 bp, Photorhabdus asymbiotica
  source 1..423: mol_type = genomic DNA
atggaaaatcaaatactgacacaactctatggtcgtggttgggcttttcctccggtcttttcccttgaaaagggggtagagatggctgaaggggcggaagatgtgagacaaagtttgcagattctgtttagtactgagccgggggaacgtcttatgcgtgaaaattatggctgcggattaaatgattttatgtttgaaaatatccgcaatgaacttattgctgaaattgaatcccatatccatgacaacgtattacgatatgaaccccgggctgatatgactgatattcaggttcgtcaatcccctggcatggggaatactttgcaagtgcaggtcatgtatcgcctgagagggagtgatatcaatcaacaaatccagggagtacttgcactgagtgaaggccgggtgacggaggtagtatga

SEQ ID NO: 71 - protein, 140 aa, Photorhabdus asymbiotica
MENQILTQLYGRGWAFPPVFSLEKGVEMAEGAEDVRQSLQILFSTEPGERLMRENYGCGLNDFMFENIRNELIAEIESHIHDNVLRYEPRADMTDIQVRQSPGMGNTLQVQVMYRLRGSDINQQIQGVLALSEGRVTEVV

SEQ ID NO: 72 - DNA, 1716 bp, Photorhabdus asymbiotica
  source 1..1716: mol_type = genomic DNA
atgacttcggagccaaatctgttaaaccggattacaattactattgaagctaataatcaacaagtagctagaaaagtattgcatggctccttgcttaatcaagctaatataaataaattatttaattcatactttaatgaatatgaaattaataggggtgtttatttagaaacattaatcctgaatcttggtacgataaatttccatgattttaattcattgtttcctactctcctaaaagctgcattgaataaagaattcagtcaatatcagataaacaaccatagggaagaaatgctatttaatgagacaatatcaaatcaagctactgataagtcttacatatttggcgataacaaattaattgatgcagagaatttcattcactttttatatcaaaagcattccacattaaatctagtagaagcaatgggaaataatggtattgaaaaattaacaaatcagttaacacaaatagaaaataaatttgcgttattattggcaaaaagttgtttgtctgaggaaggcttaaaacgactcttggctatcaaacaacccgatttattaatcgctatcaatcgcagattatctgaaagaataaatagaccacaatatcaggagaagcttgtttcctgcggacaactgatatttagtgctctgggatatatacaacagtacaatatacaggaaattcctaaaccggatgaaaaagttattgcacgcataacaactgaacttaataataatggtttgcttaatacaatacctattattacactatttcgtcagagtgggattaacgattcatcactaaatgattggctaaagaaaatctggcaggtgagatcaatttcacagttatgcagaaagtatctttctgctaaggaataccaatatctgtcagaacattttgtttcaaagagcgtcgataaaaatagatatgatgaagagcccgtaaatcagagcatattatcaaggttgaataataattccattaaagaaggaaataatcacagtcaactctgtactctcagtagactatattctgaacccgttgtattacctgaacaaaccattctacgtcaggttagtaatacagtagatcagagcatattatcaaggttgaataatgcctccattaaagaaggaaataaccaaagtcaacttcgcactctcagtagactatattctgagcccgttgcattacctgaacaaaccattccacgtcaggttagtaatacaggtatattaattctatggccaatgctacctacactatttaaccagcttggtctacttgagaaaaagaaatttatccatcgtcaggcccagtttaatgccgttgattttcttgattacctgatttggggaaccgaagatgtgaaagtggaacgaaaggttttgaataatgttctatgtgggttaatggctgatgaaattactgaaccaatgcctattgaaccagaaaaacaatggataataattcaatggctggacgctattatctcccaactttctggctggaaaaagttaagtcgtaatgacgtccgtcaattatttctacaacgaccaggagaattactgatcaatgaacaggaaattaaaatcacaatacagcaacaaccatttgatgctctgttaactgattggccgtggccaatgaatatggcttgttttagctggttgagtcaaccattaaccattacgtggttataa

SEQ ID NO: 73 - protein, 571 aa, Photorhabdus asymbiotica
MTSEPNLLNRITITIEANNQQVARKVLHGSLLNQANINKLFNSYFNEYEINRGVYLETLILNLGTINFHDFNSLFPTLLKAALNKEFSQYQINNHREEMLFNETISNQATDKSYIFGDNKLIDAENFIHFLYQKHSTLNLVEAMGNNGIEKLTNQLTQIENKFALLLAKSCLSEEGLKRLLAIKQPDLLIAINRRLSERINRPQYQEKLVSCGQLIFSALGYIQQYNIQEIPKPDEKVIARITTELNNNGLLNTIPIITLFRQSGINDSSLNDWLKKIWQVRSISQLCRKYLSAKEYQYLSEHFVSKSVDKNRYDEEPVNQSILSRLNNNSIKEGNNHSQLCTLSRLYSEPVVLPEQTILRQVSNTVDQSILSRLNNASIKEGNNQSQLRTLSRLYSEPVALPEQTIPRQVSNTGILILWPMLPTLFNQLGLLEKKKFIHRQAQFNAVDFLDYLIWGTEDVKVERKVLNNVLCGLMADEITEPMPIEPEKQWIIIQWLDAIISQLSGWKKLSRNDVRQLFLQRPGELLINEQEIKITIQQQPFDALLTDWPWPMNMACFSWLSQPLTITWL

SEQ ID NO: 74 - DNA, 2097 bp, Photorhabdus asymbiotica; identical to SEQ ID NO: 32
  source 1..2097: mol_type = genomic DNA

SEQ ID NO: 75 - protein, 698 aa, Photorhabdus asymbiotica; identical to SEQ ID NO: 31

SEQ ID NO: 76 - protein, 9 aa, synthetic construct
GGSGGSGGS

SEQ ID NO: 77 - protein, 7 aa, synthetic construct
  SITE 1: note = D-Amino acid
  SITE 2: note = D-Amino acid
  SITE 3: note = D-Amino acid
  SITE 4: note = D-Amino acid
  SITE 5: note = D-Amino acid
  SITE 6: note = D-Amino acid
  SITE 7: note = D-Amino acid
PGKLVYA

SEQ ID NO: 78 - protein, 10 aa, synthetic construct
GGSGGGGSGG

SEQ ID NO: 79 - protein, 5 aa, synthetic construct
GGSGG

(19) United States
(12) Patent Application Publication
(10) Pub. No.: US 2025/0057956 A1
(43) Pub. Date: Feb. 20, 2025

(54) CELL-TYPE SPECIFIC TARGETING CONTRACTILE INJECTION SYSTEM

(71) Applicants: The Broad Institute, Inc., Cambridge, MA (US); Massachusetts Institute of Technology, Cambridge, MA (US)

(72) Inventors: Feng ZHANG, Cambridge, MA (US); Joseph KREITZ, Cambridge, MA (US)

(73) Assignees: The Broad Institute, Inc., Cambridge, MA (US); Massachusetts Institute of Technology, Cambridge, MA (US)

(21) Appl. No.: 18/802,470

(22) Filed: Aug. 13, 2024

Related U.S. Application Data
(63) Continuation of application No. PCT/US2022/052867, filed on Dec. 14, 2022.
(60) Provisional application No. 63/310,327, filed on Feb. 15, 2022.

(51) Int. Cl. A61K 47/42; C07K 14/195

(52) U.S. Cl. CPC A61K 47/42; C07K 14/195

(57) ABSTRACT

The present disclosure relates generally to the field of delivery systems using contractile injection systems (CIS). Specifically disclosed are engineered extracellular CISs (eCISs) that can deliver non-natural protein payloads to non-natural target cells such as human cells. In addition, methods of using the engineered eCISs are also disclosed.

### CROSS REFERENCE TO RELATED APPLICATION

This application is a continuation of International Application No. PCT/US2022/052867, filed Dec. 14, 2022, which claims the benefit under 35 U.S.C. § 119(e) of U.S. Provisional Application No. 63/310,327, filed Feb. 15, 2022, the entire contents of which are incorporated herein by reference.

### STATEMENT AS TO FEDERALLY SPONSORED RESEARCH

This invention was made with government support under Grant Nos. HL141201 and HG009761 awarded by The National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Nov. 13, 2024, is named 114203-6140_SL.xml, and is 107,086 bytes in size.

### BACKGROUND

The present disclosure relates generally to the field of delivery systems using contractile injection systems (CIS).

The ability to deliver therapeutic proteins to target cells in an efficient and targeted manner would substantially advance the treatment of a wide array of diseases. However, in contrast to nucleic acid-based genetic therapies, which have benefited from years of progress in viral vector design, efficient protein delivery has proven challenging.

There has recently been an explosion of novel molecular technologies aimed at treating disease, for example in gene editing, targeted cancer therapies, immunotherapy, and others. However, one common challenge associated with these technologies is the necessity to deliver them into target cells. The usual way this has been achieved in the past is via the use of viral vectors due to their excellent transfer efficiency and targeting specificity, however these tools have presented limitations including problems with immunogenicity, cytotoxicity, concerns about off-target genomic integration, and in some cases constraints on payload size. One possible alternative to the use of viral vectors and other nucleic-acid delivery techniques is to deliver therapeutic proteins into cells directly. This idea poses a number of advantages: (1) because protein delivery tools do not involve nucleic acid, they do not pose a risk of nonspecific integration into the host genome; (2) protein delivery tools do not require target cells to be translationally active because they deliver the primary therapeutic agent instead of a DNA or RNA blueprint; therefore they may be more effective in non-proliferative or quiescent cell types that have proven difficult to target by viral vectors previously; and (3) in contrast to nucleic acid delivery, which allows target cells to produce an undefined amount of therapeutic agent, protein delivery techniques introduce defined therapeutic doses and could thus produce fewer side effects and less inter-patient variability. Protein delivery has remained challenging due to problems associated with poor delivery efficiency and specificity compared to viral vectors, reduced therapeutic potency, short therapeutic half-life, and difficulties accommodating diverse payloads with varying lengths, structures, and chemical properties.

For symbiotic bacteria living within multicellular organisms, there is a strong pressure to secrete factors that modulate host biology in favor of symbiont fitness. However, introducing complex phenotypes in host organisms often requires the action of large effector proteins that do not readily pass through membranes. This has driven the evolution of exquisite delivery systems that actively pass protein payload through eukaryotic membranes. One example is the extracellular contractile injection systems (eCISs), a class of protein delivery systems thought to have evolved from bacteriophage tail structures. eCISs are syringe-like macromolecular complexes that bind to target cells and inject protein payloads by forcefully driving a spike through the cellular membrane.

Contractile injection systems (CISs), which are thought to be evolutionarily related to bacteriophage tails, are syringe-like macromolecular complexes containing a rigid tube structure housed in a contractile sheath, which is anchored to a baseplate structure and sharpened by a spike protein. Payloads are thought to either load into the lumen of the inner tube behind the spike or associate with the spike itself, which-upon target cell recognition-is forced through the membrane via sheath contraction. This strategy has proven remarkably successful across the biosphere, as CISs have been shown to target organisms from all three domains of life. CISs can be anchored to the bacterial membrane, resulting in a contact-dependent protein delivery system known as the type VI secretion system (T6SS), or can be produced as free complexes (eCISs) and released extracellularly to deliver payloads independent of the bacterial producer. eCIS effectors have a variety of natural functions, including modulation of the host cytoskeleton, DNA cleavage, and even host toxicity. Recently, eCISs have been found to target murine cells, raising the possibility that these systems could potentially be harnessed as protein delivery systems. However, eCIS activity has yet to be shown in human cells, and the mechanisms by which eCISs recruit payload proteins and bind to target cells-both necessary preconditions for the delivery of arbitrary payloads into defined cell types and tissues-remains to be elucidated.

### SUMMARY OF THE INVENTION

An aspect of the disclosure is directed to an engineered extracellular contractile injection system (eCIS) comprising: a structural domain forming a tubular structure; a targeting domain engineered to target the eCIS to a eukaryotic cell of interest; and a protein payload, wherein the targeting domain is capable of targeting the assembled eCIS to the eukaryotic cell of interest.

In some embodiments, the structural domain comprises a tube and a sheath enclosing the tube, a baseplate located at a first end of the tube enclosed by the sheath, and a terminal cap located at a second end of the tube enclosed by the sheath.

In some embodiments, the tube comprises about 10-40 layers of a tube protein and a plurality of tube connector proteins that connect the tube to the baseplate, and wherein the sheath comprises about 10-40 layers of a sheath protein and a plurality of sheath connector proteins that connect the sheath to the baseplate, and wherein the baseplate comprises a plurality of baseplate proteins, and a plurality of spike proteins, and wherein the terminal cap comprises a terminal cap protein.

In some embodiments, the tube protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 1, or between about 80% to 100% sequence identity to SEQ ID NO: 1, or the tube protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 2, or between about 80% to 100% sequence identity to SEQ ID NO: 2.

In some embodiments, the plurality of tube connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 3 or 5, or between about 80% to 100% sequence identity to SEQ ID NOs: 3 or 5, or the plurality of tube connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 4 or 6, or between about 80% to 100% sequence identity to SEQ ID NOs: 4 or 6.

In some embodiments, the sheath protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 7, or between about 80% to 100% sequence identity to SEQ ID NO: 7, or the sheath protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 8, or between about 80% to 100% sequence identity to SEQ ID NO: 8.

In some embodiments, the plurality of sheath connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 9 or 11, or between about 80% to 100% sequence identity to SEQ ID NOs: 9 or 11, or the plurality of sheath connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: or 12, or between about 80% to 100% sequence identity to SEQ ID NOs: 10 or 12.

In some embodiments, the plurality of baseplate proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 13 or 15, or between about 80% to 100% sequence identity to SEQ ID NOs: 13 or 15, or the plurality of baseplate proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 14 or 16, or between about 80% to 100% sequence identity to SEQ ID NOs: 14 or 16.

In some embodiments, the plurality of spike proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 19 or 21, or between about 80% to 100% sequence identity to SEQ ID NOs: 19 or 21, or the plurality of spike proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 20 or 22, or between about 80% to 100% sequence identity to SEQ ID NOs: 20 or 22.

In some embodiments, the terminal cap protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO:23, or between about 80% to 100% sequence identity to SEQ ID NO:23, or the terminal cap protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 24, or between about 80% to 100% sequence identity to SEQ ID NO: 24.

In some embodiments, the targeting domain comprises a tail fiber protein fused to a heterologous binding moiety, wherein the tail fiber comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 17, or between about 80% to 100% sequence identity to SEQ ID NO: 17, or the tail fiber is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 18, or between about 80% to 100% sequence identity to SEQ ID NO: 18.

In some embodiments, the heterologous binding moiety is selected from the group consisting of an antibody, an antibody fragment, a viral receptor-binding domain, an artificial receptor, a protein tag, and a tail fiber from an orthologous eCIS. In some embodiments, the antigen binding fragment is selected from the group consisting of a Fab, a Fab′, a F(ab′)2, an Fd, an Fv, a domain antibody (dAb), a complementarity determining region (CDR), a single chain variable fragment antibody (scFv), a maxibody, a minibody, an intrabody, a diabody, a triabody, a tetrabody, a v-NAR and a bis-scFvs. In some embodiments, the dAb is a shark antibody or a camelid antibody.

In some embodiments, the eukaryotic cell of interest is a yeast cell, an insect cell, a mammalian cell, a plant cell or a fungi cell. In some embodiments, the mammalian cell is a human cell, a mouse cell, a rat cell, a cat cell, a dog cell, a horse cell, a sheep cell, a cow cell or a pig cell. In some embodiments, the human cell is a cancerous cell.

In some embodiments, the payload comprises an N-terminal packaging domain, wherein the N-terminal packaging domain is between 38 and 67 amino acids long. In some embodiments, the N terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 25, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 26, or between about 80% to 100% sequence identity to SEQ ID NO: 26. In some embodiments, the N-terminal packaging domain comprises SEQ ID NO: 26.

In some embodiments, the protein payload is between 5 and 2000 or between 10 and 1000 amino acids long. In some embodiments, the protein payload is a gene editor, a chemotherapy agent or a toxin. In some embodiments, the gene editor is a Cre protein, a Cas protein, a Transcription activator-like effector nuclease (TALEN) or a Zing finger nuclease (ZFN).

In some embodiments, the eCIS is derived from Photorhabdus asymbiotica virulence cassette (PVC), Serratia entomophila anti-feeding prophage (AFP), Pseudoalteromonas luteoviolacea metamorphosis-associated contractile structure (MAC), Amoebophilus asiaticus T6SSiv, Pseudomonas aeruginosa T6SS, or Pseudomonas aeruginosa R-type bacteriocin. In some embodiments, the eCIS is derived from a Subtype Ia eCIS locus, a Subtype Ib eCIS locus, a Subtype IIa eCIS locus, a Subtype IIb eCIS locus, a Subtype IIc eCIS locus, or a Subtype IId eCIS locus.

Another aspect of the disclosure is directed to a vector system encoding the engineered eCIS described herein.

Another aspect of the disclosure is directed to a method for protein delivery comprising administering an engineered extracellular contractile injection system (eCIS) to a cell, the eCIS comprising: a structural domain; a targeting domain engineered to target the eCIS to an eukaryotic cell of interest; and a protein payload, wherein the targeting domain is capable of targeting the assembled eCIS to the eukaryotic cell of interest.

In some embodiments, the structural domain comprises a tube and a sheath enclosing the tube, a baseplate located at a first end of the tube enclosed by the sheath, and a terminal cap located at a second end of the tube enclosed by the sheath.

In some embodiments, the tube comprises about 10-40 layers of a tube protein and a plurality of tube connector proteins that connect the tube to the baseplate, and wherein the sheath comprises about 10-40 layers of a sheath protein and a plurality of sheath connector proteins that connect the sheath to the baseplate, and wherein the baseplate comprises a plurality of baseplate proteins, and a plurality of spike proteins, and wherein the terminal cap comprises a terminal cap protein.

In some embodiments, the tube protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 1, or between about 80% to 100% sequence identity to SEQ ID NO: 1, or the tube protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 2, or between about 80% to 100% sequence identity to SEQ ID NO: 2.

In some embodiments, the plurality of tube connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 3 or 5, or between about 80% to 100% sequence identity to SEQ ID NOs: 3 or 5, or the plurality of tube connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 4 or 6, or between about 80% to 100% sequence identity to SEQ ID NOs: 4 or 6.

In some embodiments, the sheath protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 7, or between about 80% to 100% sequence identity to SEQ ID NO: 7, or the sheath protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 8, or between about 80% to 100% sequence identity to SEQ ID NO: 8.

In some embodiments, the plurality of sheath connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 9 or 11, or between about 80% to 100% sequence identity to SEQ ID NOs: 9 or 11, or the plurality of sheath connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: or 12, or between about 80% to 100% sequence identity to SEQ ID NOs: 10 or 12.

In some embodiments, the plurality of baseplate proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 13 or 15, or between about 80% to 100% sequence identity to SEQ ID NOs: 13 or 15, or the plurality of baseplate proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 14 or 16, or between about 80% to 100% sequence identity to SEQ ID NOs: 14 or 16.

In some embodiments, the plurality of spike proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 19 or 21, or between about 80% to 100% sequence identity to SEQ ID NOs: 19 or 21, or the plurality of spike proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 20 or 22, or between about 80% to 100% sequence identity to SEQ ID NOs: 20 or 22.

In some embodiments, the terminal cap protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO:23, or between about 80% to 100% sequence identity to SEQ ID NO:23, or the terminal cap protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 24, or between about 80% to 100% sequence identity to SEQ ID NO: 24.

In some embodiments, the targeting domain comprises a tail fiber protein fused to a heterologous binding moiety, wherein the tail fiber comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 17, or between about 80% to 100% sequence identity to SEQ ID NO: 17, or the tail fiber is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 18, or between about 80% to 100% sequence identity to SEQ ID NO: 18.

In some embodiments, the heterologous binding moiety is selected from the group consisting of an antibody, an antibody fragment, a viral receptor-binding domain, an artificial receptor, a protein tag, and a tail fiber from an orthologous eCIS. In some embodiments, the antigen binding fragment is selected from the group consisting of a Fab, a Fab′, a F(ab′)2, an Fd, an Fv, a domain antibody (dAb), a complementarity determining region (CDR), a single chain variable fragment antibody (scFv), a maxibody, a minibody, an intrabody, a diabody, a triabody, a tetrabody, a v-NAR and a bis-scFvs. In some embodiments, the dAb is a shark antibody or a camelid antibody.

In some embodiments, the eukaryotic cell of interest is a yeast cell, an insect cell, a mammalian cell, a plant cell or a fungi cell. In some embodiments, the mammalian cell is a human cell, a mouse cell, a rat cell, a cat cell, a dog cell, a horse cell, a sheep cell, a cow cell or a pig cell. In some embodiments, the human cell is a cancerous cell.

In some embodiments, the payload comprises an N-terminal packaging domain, wherein the N-terminal packaging domain is between 38 and 67 amino acids long. In some embodiments, the N terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 25, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 26, or between about 80% to 100% sequence identity to SEQ ID NO: 26. In some embodiments, the N-terminal packaging domain comprises SEQ ID NO: 26.

In some embodiments, the protein payload is between 5 and 2000 or between 10 and 1000 amino acids long. In some embodiments, the protein payload is a gene editor, a chemotherapy agent or a toxin. In some embodiments, the gene editor is a Cre protein, a Cas protein, a Transcription activator-like effector nuclease (TALEN) or a Zing finger nuclease (ZFN).

In some embodiments, the eCIS is derived from Photorhabdus asymbiotica virulence cassette (PVC), Serratia entomophila anti-feeding prophage (AFP), Pseudoalteromonas luteoviolacea metamorphosis-associated contractile structure (MAC), Amoebophilus asiaticus T6SSiv, Pseudomonas aeruginosa T6SS, or Pseudomonas aeruginosa R-type bacteriocin. In some embodiments, the eCIS is derived from a Subtype Ia eCIS locus, a Subtype Ib eCIS locus, a Subtype IIa eCIS locus, a Subtype IIb eCIS locus, a Subtype IIc eCIS locus, or a Subtype IId eCIS locus.

In some embodiments, the protein payload is encoded by a nucleic acid in a separate vector.

### BRIEF DESCRIPTION OF THE DRAWINGS

FIGS. 1A-1H. (A) Genomic structure of a PVC locus. Endogenous P. asymbiotica PVCpnf locus contains 16 structural genes followed by two payload genes (SepC and Pnf) as well as several regulatory genes. (B) Engineering PVCs for expression in E. Coli. (C)-(D) PVCs can be purified from E. coli. The PVCpnf locus was cloned into an expression backbone and transformed into E. coli EPI300. The cells were grown at 30° C. for 24 h before being lysed in a buffer containing 5% Triton X-100. PVC protein was purified by ultracentrifugation and analyzed with (C) Coomassie stain and (D) TEM imaging. (E)-(F) Purified PVC particles kill cultured insect cells. Sf9 cells were cultured at 28° C. for 24 h before being exposed to lug of purified PVC protein. The cells were imaged at t=4 days using FDA/PI live/dead staining, and cytotoxicity was quantified using CellTiter-Glo viability assays. (G) Tail fiber mutant PVCs can still load endogenous (SepC or Pnf) or arbitrary (Cre) payloads. (H) Purified payloads (administered independent of PVCs) cannot kill Sf9 cells.

FIGS. 2A-2F. PVCs can be reprogrammed to load and inject novel proteins into target cells. (A) Schematic representation of the payload loading assay. Cultures containing both unloaded and loaded payload proteins were spun in an ultracentrifuge and only assembled PVC particles localized to the pellet. This allowed the identification of loaded payload proteins via denaturing western blot. (B) Upon an Alphafold analysis, N terminal domain comprising the putative packaging domain was found to be highly disordered (left). (C) PVC payloads were serially truncated to locate minimal sequences capable of packaging the payload into a PVC particle (right). A ˜60 residue motif on the N-terminus of the payload protein was sufficient for loading, and truncation of this motif inhibited loading. (D) Arbitrary payloads can be loaded into PVC particles. Packaging domains from (C) were fused to novel proteins (Cre, GFP, and a zinc finger nuclease monomer), and packaging was determined via western blot. (E) PVCs can also load TALENs as payload. (F) PVCs can inject novel proteins into target cells. PVCs loaded with Cre were exposed to cultured Sf9 cells, and injection was assessed via co-transfection of a double-floxed EGFP plasmid.

FIGS. 3A-3H. (A) PVCs can be reprogrammed to target human cells. The putative target cell recognition domain of pvc13 was truncated and replaced with either a host-binding domain from a human virus (hAd5 knob), or an artificial binding protein targeting a human receptor (anti-EGFR DARPin E01). (B) A human cell line (A549) was selected as the target for this experiment as it is known to overexpress EGFR and can be targeted by hAd5 virus. PVC particles were loaded with Cre, and PVC-driven protein delivery was measured as GFP expression. (C) eCISs can efficiently kill cells. PVC particles were loaded with endogenous PVC toxin payloads (pvc17+pvc21), and PVC-driven protein delivery was measured as cytotoxicity, respectively. A human cell line (A549) was selected as the target for this experiment as it is known to overexpress EGFR and can be targeted by hAd5 virus. Cytotoxicity was visualized with FDA/PI stain and quantified using Celltiter-Glo viability assay. (D) Quantification viability of high EGFR-expressing A549 cells shown in (C). (E) Quantification viability of low EGFR-expressing HEK293T cells. Less activity with anti-EGFR DARPin is observed in a cell line with less EGFR expression. (F) PVCs can target cells displaying artificial “receptors.” PVC-mediated delivery can be mediated by an engineered receptor-binding interaction. (G) HEK293T cells transfected with construct that encode various antibodies (anti-HA, anti-FLAG, anti-EE, anti-MoonTag, anti-SunTag, and anti-ALFA tag scFv) were exposed to PVC particles targeted with tail fibers harboring different tags and delivery was detected as Cre-driven GFP. GFP expression was observed only PVC particles with tags matching the antibody expressed on the HEK293T cells. (H) Additional technical details about eCIS engineering. Wild type PVCs prefer to target insect cells rather than human cells. On the other hand, PVCs that lack the C terminal domain (ACTD) show aberrant GFP signal, possibly due to sporadic payload ejection. When the receptor binding domain was deleted (AReceptor binding domain), the aberrant signal was abolished, presumably because the PVCs no longer targeted HEK293T cells. When a novel binding domain was added, the GFP signal reaapeared in the cells, whereas a mutant version of the novel binding domain was not sufficient to bring back the GFP signal, demonstrating the specificity of targeting. Figure discloses SEQ ID NOS 78-79, 79, 79, and 79, respectively, in order of appearance.

FIG. 4. PVCs can be visualized binding to target cells. PVC particles containing sheath proteins (pvc2) with N-terminal FLAG tags were incubated with Sf9 cells for 24 hr and binding was visualized via immunofluorescence.

FIGS. 5A-5B. eCIS-mediated delivery of base editors into human cells. (A) Zinc finger deaminase (ZFD) was delivered to H1EK293T cells using eCIS. The cells trated with the DL491-492 mutant eCIS (which cannot bind to target cells) did not display base editing. (B) spCas9 was delivered to HEK293T cells using eCIS. Successful indel production was observed when Cas9 was delivered together with a guide RNA (guide). The cells trated with the DL491-492 mutant eCIS (which cannot bind to target cells) did not display any indels.

FIGS. 6A-6B. New eCIS designs enable delivery of cargo to mouse cells. (A) Different eCIS designs: pvc13 with WT tail fiber (pvc13(WT)), pvc13 with Ad5 binding domain having RGD and polylysine (PK7) domains (pvc13-Ad5 knob (RGD/PK7), which shows enhanced targeting properties, and pvc13 with anti-mouse MHC II nanobody (pvc13-anti-mouse MHC II Nb). (B) Cytotoxicity assay using different eCIS constructs. Briefly, pvc13 with WT and truncated tail fiber domains were unable to target and kill human or mouse cells. Ad5 knob-targeted pvc13 were able to target human A549 cells; but they could only target and kill N2a mouse cells. On the other hand, super infective Ad5(RGD/PK7)-targeted pvc13 was able to target all cells tested. Mouse-cell specific targeting was observed using pvc13-anti-mouse MHC II Nb which targeted and killed MHCII expressing A20 cells and primary splenocytes. Mouse-cell specific targeting was not onserved with pvc13 having a nontargeting nanobody (pvc13-Nontargeting Nb).

FIGS. 7A-7B. The new eCIS designs are active in vivo. (A) Schematic representation of the experiment. Mouse targeting PVCs loaded with Cre were administered to LoxP-T dTomato mice via stereotaxic brain injection. 12 days later mice were sacrificed and their brain sections were imaged for TdTomato expression. (B) TdTomato expression was observed in mouse brains treated with pvc13-Ad5 knob (RGD/PK7), but not in mouse brains treated with a pvc13 version lacking the pvc10 structural gene encoding the spike protein (pvc13-Ad5 knob (RGD/PK7) Apvc10).

FIGS. 8A-8C. eCISs are highly specific. (A) eCISs that have truncated tails (pvc13(truncated)) are unable to target and kill any cell line tested. On the other hand, eCISs having an EGFR-specific targeting domain (pvc13-E01 DARPin) are able to target and kill cell lines that have high EGFR expression (e.g., A431 and A549) more than cell lines that have low EGFR expression (e.g., Jurkat and 3T3). (B) eCISs having an EGFR-specific targeting domain (pvc13-E01 DARPin) are able to target and kill 3T3 cell line which are transfected with an EGFR construct. (C) Jurkat cells, which express high levels of EGFR, are only targeted and killed by eCISs expressing a targeting domain specific to a protein they express (CD4 in this instance).

FIG. 9. Graphical representation of eCIS action. An eCIS binds to the target cell through the modified targeting domain. The eCIS then contracts and injects its custom payload into the target cell.

### DETAILED DESCRIPTION

### Descriptions

As used herein, the term “about” refers to +10% of a given value.

### Engineered Extracellular Contractile Injection Systems

An aspect of the disclosure is directed to an engineered extracellular contractile injection system (eCIS) comprising: a structural domain forming a tubular structure; a targeting domain engineered to target the eCIS to a eukaryotic cell of interest; and a protein payload, wherein the targeting domain is capable of targeting the assembled eCIS to the eukaryotic cell of interest.

In some embodiments, the eCIS of the instant disclosure is engineered from a bacterial tailocin or a bacterial pyocin as described in Ge et al. (Nature structural & molecular biology 22.5 (2015): 377-382); Ghequire and De Mot (Trends in microbiology 23.10 (2015): 587-590); Nakayama et al., 2000 (Molecular microbiology 38.2 (2000): 213-231); and Scholl, D. (Annual Review of Virology 4 (2017): 453-467), which are incorporated herein in their entireties.

In some embodiments, the eCIS of the instant disclosure is engineered from an eCIS of prokaryotic or archaeal origin as the eCISs described in Sarris et al., (Genome Biology and Evolution 6.7 (2014): 1739-1747), which is incorporated herein in its entirety. In some embodiments, the eCIS is engineered from a Photorhabdus virulence cassette (PVC) as described in Yang et al. (J. Bacteriol. (2006), 188, 2254-2261), which is incorporated herein in its entirety. In some embodiments, the eCIS is engineered from an antifeeding prophage (Afp) as described in Heymann et al. (Journal of Biological Chemistry 288.35 (2013): 25276-25284); Hurst et al. (Journal of bacteriology 186.15 (2004): 5116-5128); and Jank et al., (Nature communications 6.1 (2015): 1-7), which are incorporated herein in their entireties. In some embodiments, the eCIS is engineered from a metamorphosis-associated contractile structure (MAC) as described in Shikuma et al. (Science 343.6170 (2014): 529-533), which is incorporated herein in its entirety. In some embodiments, the eCIS of the instant disclosure is engineered from an eCIS described in Chen et al. (Cell reports 29.2 (2019): 511-521).

In some embodiments, the structural domain comprises a tube and a sheath enclosing the tube, a baseplate located at a first end of the tube enclosed by the sheath, and a terminal cap located at a second end of the tube enclosed by the sheath.

In the eCIS of this disclosure, there are equal or almost equal number of layers of proteins in the tube and the sheath. In other words, the sheath covers the tube such that their length are equal, or almost equal.

In some embodiments, the tube comprises about 10-40 layers of a tube protein and a plurality of tube connector proteins that connect the tube to the baseplate, and wherein the sheath comprises about 10-40 layers of a sheath protein and a plurality of sheath connector proteins that connect the sheath to the baseplate, and wherein the baseplate comprises a plurality of baseplate proteins, and a plurality of spike proteins, and the terminal cap comprises a terminal cap protein. In some embodiments, the tube comprises about 5-100 layers of a tube protein and a plurality of tube connector proteins that connect the tube to the baseplate, and wherein the sheath comprises about 5-100 layers of a sheath protein and a plurality of sheath connector proteins that connect the sheath to the baseplate, and wherein the baseplate comprises a plurality of baseplate proteins, and a plurality of spike proteins, and wherein the terminal cap comprises a terminal cap protein.

In some embodiments, the structural domain comprises about 10-40 or about 5-100 layers of tube protein enclosed in a similar number of sheath proteins, and is terminated by a cap protein. In some embodiments, the sheath comprises three different sheath proteins (pvc2-4) and the tube (which exists inside the sheath) is composed of one tube protein (pvc1). In some embodiments, the tube is primarily composed of repeating pvc1 proteins, but is also initiated with two slightly different tube proteins (pvc5/pvc7) which connect the tube to the baseplate. In some embodiments, the sheath is primarily composed of repeating pvc2 proteins, but also uses pvc3 and pvc4 near the baseplate.

In some embodiments, the structural domain further comprises a spike that punctures a target cell membrane. In some embodiments, the spike is a complex of Pvc8/Pvc10. In some embodiments, the structural domain further comprises a baseplate that stabilizes the overall structure and houses the spike. In some embodiments, the baseplate is a complex of Pvc11 and Pvc12. In some embodiments, the targeting domain comprises a tail fiber which protrudes from the baseplate and binds the eCIS to a target cell.

In some embodiments, the targeting domain comprises a heterologous binding moiety inserted/fused into the distal portion of the wildtype eCIS tail fiber protein. In some embodiments, the tail fiber comprises Pvc13.

In some embodiments, the tube comprises about 5, 10, 15, 20, 25, 30, 35, 40, 45, 50, 55, 60, 65, 70, 75, 80, 85, 90, 95 or 100 layers of a tube protein. In some embodiments, the sheath comprises about 5, 10, 15, 20, 25, 30, 35, 40, 45, 50, 55, 60, 65, 70, 75, 80, 85, 90, 95 or 100 layers of a sheath protein.

In some embodiments, the tube protein comprises the Pvc1 protein of Photorhabdus. In some embodiments, the tube protein comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO: 1, or between about 80% to 100% sequence identity to SEQ ID NO: 1, or the tube protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 2, or between about 80% to 100% sequence identity to SEQ ID NO: 2.

In some embodiments, the plurality of tube connector proteins comprises the Pvc5 and/or Pvc7 protein of Photorhabdus. In some embodiments, the plurality of tube connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 3 and/or 5, or between about 80% to 100% sequence identity to SEQ ID NOs: 3 and/or 5, or the plurality of tube connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 4 and/or 6, or between about 80% to 100% sequence identity to SEQ ID NOs: 4 and/or 6.

In some embodiments, the sheath protein comprises the Pvc2 protein of Photorhabdus. In some embodiments, the sheath protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 7, or between about 80% to 100% sequence identity to SEQ ID NO: 7, or the sheath protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 8, or between about 80% to 100% sequence identity to SEQ ID NO: 8.

In some embodiments, the plurality of sheath connector proteins comprises the Pvc3 protein and/or Pvc4 protein of Photorhabdus. In some embodiments, the plurality of sheath connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 9 and/or 11, or between about 80% to 100% sequence identity to SEQ ID NOs: 9 and/or 11, or the plurality of sheath connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 10 and/or 12, or between about 80% to 100% sequence identity to SEQ ID NOs: 10 and/or 12.

In some embodiments, the plurality of baseplate proteins comprise the Pvc11 and/or Pvc12 proteins of Photorhabdus. In some embodiments, the plurality of baseplate proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 13 and/or 15, or between about 80% to 100% sequence identity to SEQ ID NOs: 13 and/or 15, or the plurality of baseplate proteins is encoded by a nucleic acid sequence having at least about 85%, 86%, 87%, 88%, 89%, 90%, 91%, 92%, 93%, 94%, 95%, 96%, 97%, 98%, 99% and/or 100% sequence identity to SEQ ID NOs: 14 and/or 16, or between about 80% to 100% sequence identity to SEQ ID NOs: 14 and/or 16.

In some embodiments, the plurality of spike proteins comprise the Pvc8 and/or Pvc10 proteins of Photorhabdus. In some embodiments, the plurality of spike proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 19 and/or 21, or between about 80% to 100% sequence identity to SEQ ID NOs: 19 and/or 21, or the plurality of spike proteins is encoded by a nucleic acid sequence having at least about 85%, 86%, 87%, 88%, 89%, 90%, 91%, 92%, 93%, 94%, 95%, 96%, 97%, 98%, 99% and/or 100% sequence identity to SEQ ID NOs: 20 and/or 22, or between about 80% to 100% sequence identity to SEQ ID NOs: 20 and/or 22.

In some embodiments, the terminal cap protein comprises the Pvc16 protein of Photorhabdus. In some embodiments, the terminal cap protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO:23, or between about 80% to 100% sequence identity to SEQ ID NO:23, or the terminal cap protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 24, or between about 80% to 100% sequence identity to SEQ ID NO: 24.

In some embodiments, the tail fiber protein comprises the Pvc13 protein of Photorhabdus. In some embodiments, the targeting domain comprises a tail fiber protein fused, linked or attached to a heterologous binding moiety, wherein the tail fiber comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 17, or between about 80% to 100% sequence identity to SEQ ID NO: 17, or the tail fiber is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 18, or between about 80% to 100% sequence identity to SEQ ID NO: 18.

In some embodiments, the heterologous binding moiety is selected from the group consisting of an antibody, an antibody fragment, a viral receptor-binding domain, an artificial receptor, a protein tag, and a tail fiber from an orthologous eCIS. In some embodiments, the antigen binding fragment is selected from the group consisting of a Fab, a Fab′, a F(ab′)₂, an Fd, an Fv, a domain antibody (dAb), a complementarity determining region (CDR), a single chain variable fragment antibody (scFv), a maxibody, a minibody, an intrabody, a diabody, a triabody, a tetrabody, a v-NAR and a bis-scFvs. In some embodiments, the dAb is a shark antibody or a camelid antibody.

In some embodiments, the heterologous binding moiety is linked or attached to the tail fiber protein via a tag. In some embodiments, the tag is selected from the group consisting of a SNAP tag, a biotin tag, an Isopeptag, a SpyTag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag, a Myc tag, a MoonTag, a SunTag, an Alfa tag, an EE tag, a His tag, and a HiBiT-tag.

In some embodiments, the eukaryotic cell of interest is a yeast cell, an insect cell, a mammalian cell, a plant cell or a fungi cell. In some embodiments, the mammalian cell is a human cell, a mouse cell, a rat cell, a cat cell, a dog cell, a horse cell, a sheep cell, a cow cell or a pig cell. In some embodiments, the human cell is a cancerous cell. In some embodiments, the eukaryotic cell of interest is part of a tissue, an organ or a whole organism.

In some embodiments, the payload comprises an N-terminal packaging domain that is between 38 and 67 amino acids long. In some embodiments, the payload comprises an N-terminal packaging domain that is 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 amino acids long. In some embodiments, the N-terminal packaging domain comprises the first (i.e., from the N-terminal) 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 amino acids of SEQ ID NO: 25.

In some embodiments, the N-terminal packaging domain comprises N-terminal domain (NTD) of the SepC payload as shown in SEQ ID NO: 25. In some embodiments, the N-terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 25, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 26, or between about 80% to 100% sequence identity to SEQ ID NO: 26.

In some embodiments, the N-terminal packaging domain comprises N-terminal domain (NTD) of the Pnf payload as shown in SEQ ID NO: 27. In some embodiments, the N-terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 27, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 28, or between about 80% to 100% sequence identity to SEQ ID NO: 28.

In some embodiments, the protein payload is an arbitrary protein (any protein of interest). In some embodiments, the protein payload is between 5 and 2000, between 10 and 1000, between 100 and 500, between 500 and 1000, or between 1000 and 1500 amino acids long. In some embodiments, the protein payload is about 5, 10, 20, 30, 40, 50, 75, 100, 150, 200, 250, 300, 350, 400, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1850, 1900, 1950, or 2000 amino acids long. In some embodiments, the protein payload is an enzyme, a cytokine or a binding agent (e.g., an antibody fragment, a Fab, a Fab′, a F(ab′)₂, an Fd, an Fv, a domain antibody (dAb), a complementarity determining region (CDR), or a single chain variable fragment antibody (scFv)). In some embodiments, the protein payload is a gene editor, a chemotherapy agent or a toxin. In some embodiments, the gene editor is a Cre protein, a Cas protein, a Transcription activator-like effector nuclease (TALEN) or a Zing finger nuclease (ZFN). In some embodiments, the protein payload is a ribonucleoprotein (RNP). In a specific embodiment, the protein payload is a Cas RNP. In some embodiments, the eCIS system with engineered tropism is used to package and deliver a CRISPR-Cas protein or a fusion protein thereof (e.g., a base editor or prime editor). In some embodiments, the eCIS system with engineered tropism is used to package and deliver an RNP comprising a CRISPR-Cas protein or a fusion protein thereof (e.g., a base editor or prime editor), and a gRNA of the CRISPR-Cas protein.

In some embodiments, the protein payload (or “cargo protein”) is fused at its N-terminus to a packaging domain via a linker. In some embodiments, the protein payload or cargo protein is fused at its C-terminus to a packaging domain via a linker. In some embodiments, the linker is a cleavable linker. In some embodiments, cleavage of the linker in the target cell exposes a nuclear localization signal (NLS) positioned between the linker and the protein payload or cargo protein.

In some embodiments, the CRISPR-Cas protein or fusion protein thereof is fused at its N-terminus to a packaging domain via a linker. In some embodiments, the CRISPR-Cas protein or fusion protein thereof is fused at its C-terminus to a packaging domain via a linker. In some embodiments, the linker is a cleavable linker. In some embodiments, cleavage of the linker in the target cell exposes a nuclear localization signal (NLS) positioned between the linker and the CRISPR-Cas protein or fusion protein.

In some embodiments, there is a 3×GGSGG linker as shown in SEQ ID NO: 29 as a spacer between the N-terminal packaging domain and the protein payload or cargo protein.

In some embodiments, the payload is loaded into the tubular structure of the structural domain by an ATPase loader domain, thereby producing an assembled and loaded eCIS. In some embodiments, the ATPase loader domain comprises the Pvc15 protein of Photorhabdus.

In some embodiments, the ATPase loader domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 31, or between about 80% to 100% sequence identity to SEQ ID NO: 31, or the ATPase loader domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 32, or between about 80% to 100% sequence identity to SEQ ID NO: 32.

In some embodiments, the eCIS is derived from Photorhabdus asymbiotica virulence cassette (PVC), Serratia entomophila anti-feeding prophage (AFP), Pseudoalteromonas luteoviolacea metamorphosis-associated contractile structure (MAC), Amoebophilus asiaticus T6SSiv, Pseudomonas aeruginosa T6SS, or Pseudomonas aeruginosa R-type bacteriocin. In some embodiments, the eCIS is derived from a Subtype Ia eCIS locus, a Subtype Ib eCIS locus, a Subtype IIa eCIS locus, a Subtype IIb eCIS locus, a Subtype IIc eCIS locus, or a Subtype IId eCIS locus.

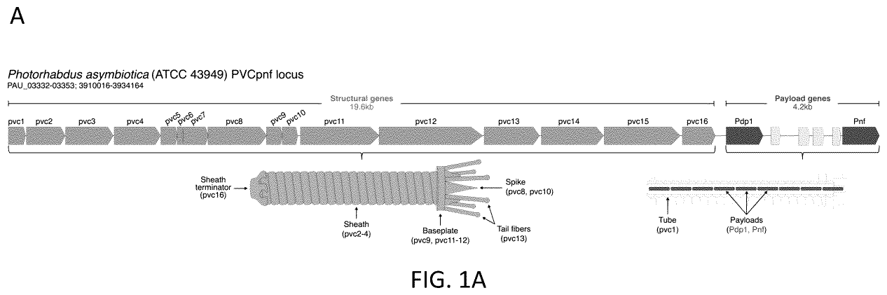

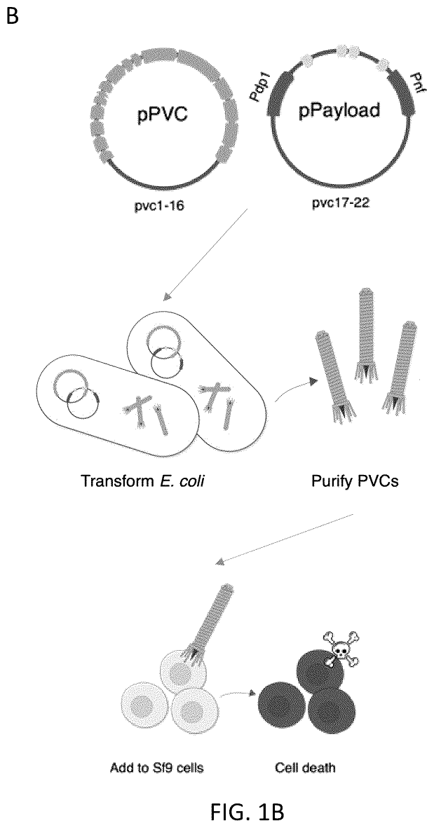

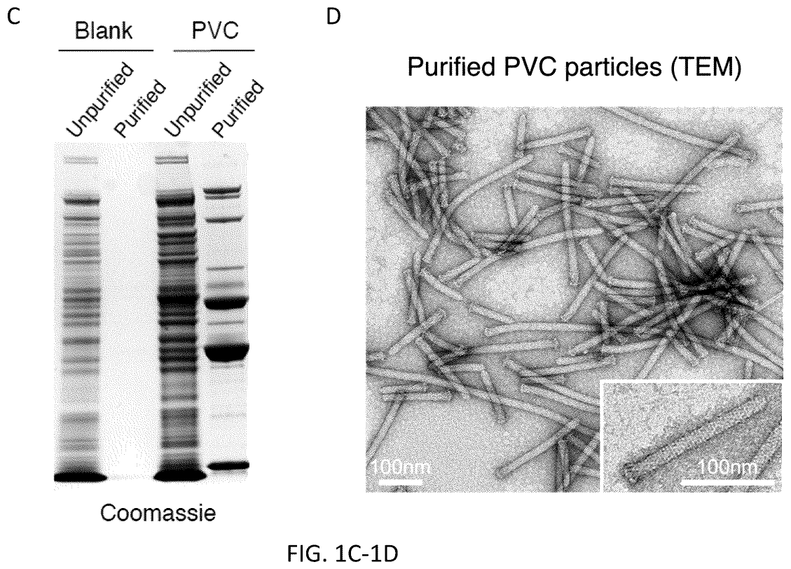

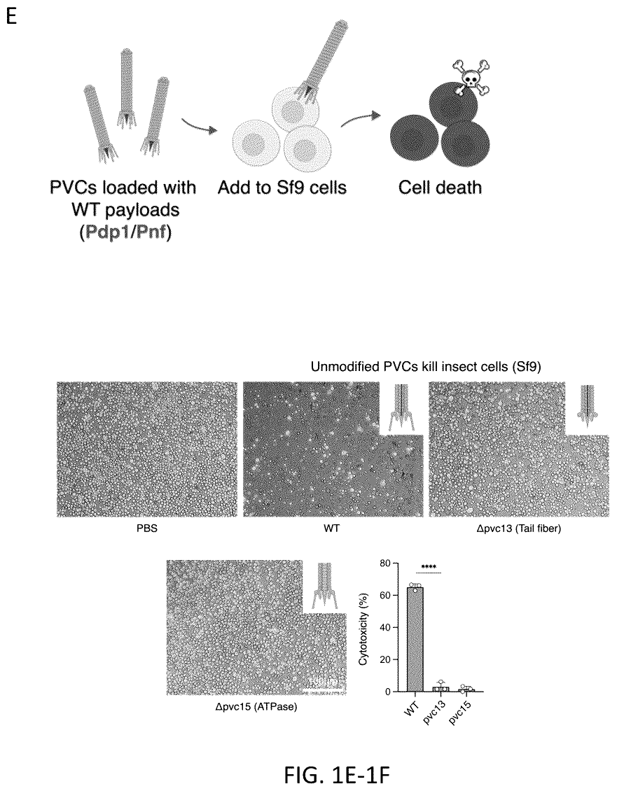

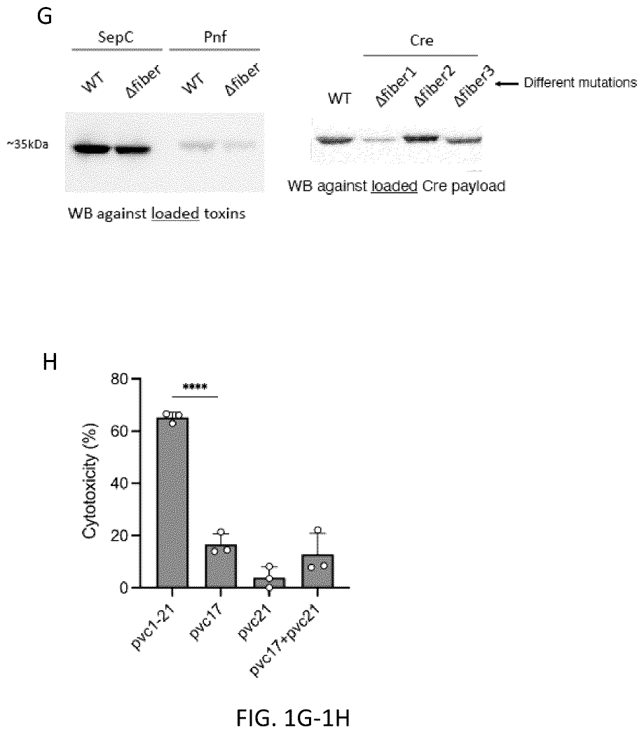

Another aspect of the disclosure relates to a vector system comprising nucleic acids encoding the engineered eCIS proteins described herein (FIG. 1). Another aspect of the disclosure relates to a vector system for producing the engineered eCIS, wherein the vector system comprises one or more vectors encoding the structural domain and the targeting domain (FIG. 1i). Another aspect of the disclosure relates to a host cell comprising or transformed with the vector system (FIG. 1i). Another aspect of the disclosure relates to a host cell for producing the engineered eCIS, wherein the host cell comprising one or more polynucleotides encoding structural domain and the targeting domain (FIG. 1). Another aspect of the disclosure relates to a method for producing the eCIS, comprising expressing one or more polynucleotides encoding the structural domain and the targeting domain in a host cell in the presence of the protein payload (FIG. 1i).

### Methods for Protein Delivery

Another aspect of the disclosure is directed to a method for protein delivery comprising administering an engineered extracellular contractile injection system (eCIS) to a cell, the eCIS comprising: a structural domain; a targeting domain engineered to target the eCIS to an eukaryotic cell of interest; and a protein payload, wherein the targeting domain is capable of targeting the assembled eCIS to the eukaryotic cell of interest.

In some embodiments, the structural domain comprises a tube and a sheath enclosing the tube, a baseplate located at a first end of the tube enclosed by the sheath, and a terminal cap located at a second end of the tube enclosed by the sheath.

In the eCIS of this disclosure, there are equal or almost equal number of layers of proteins in the tube and the sheath. In other words, the sheath covers the tube such that their length are equal, or almost equal.

In some embodiments, the tube comprises about 10-40 layers of a tube protein and a plurality of tube connector proteins that connect the tube to the baseplate, and wherein the sheath comprises about 10-40 layers of a sheath protein and a plurality of sheath connector proteins that connect the sheath to the baseplate, and wherein the baseplate comprises a plurality of baseplate proteins, and a plurality of spike proteins, and the terminal cap comprises a terminal cap protein.

In some embodiments, the tube comprises about 5-100 layers of a tube protein and a plurality of tube connector proteins that connect the tube to the baseplate, and wherein the sheath comprises about 5-100 layers of a sheath protein and a plurality of sheath connector proteins that connect the sheath to the baseplate, and wherein the baseplate comprises a plurality of baseplate proteins, and a plurality of spike proteins, and wherein the terminal cap comprises a terminal cap protein.

In some embodiments, the structural domain comprises about 10-40 or about 5-100 layers of tube protein enclosed in a similar number of sheath proteins, and is terminated by a cap protein. In some embodiments, the sheath comprises three different sheath proteins (pvc2-4) and the tube (which exists inside the sheath) is composed of one tube protein (pvc1). In some embodiments, the tube is primarily composed of repeating pvc1 proteins, but is also initiated with two slightly different tube proteins (pvc5/pvc7) which connect the tube to the baseplate. In some embodiments, the sheath is primarily composed of repeating pvc2 proteins, but also uses pvc3 and pvc4 near the baseplate.

In some embodiments, the structural domain further comprises a spike that punctures a target cell membrane. In some embodiments, the spike is a complex of Pvc8/Pvc10. In some embodiments, the structural domain further comprises a baseplate that stabilizes the overall structure and houses the spike. In some embodiments, the baseplate is a complex of Pvc11 and Pvc12. In some embodiments, the targeting domain comprises a tail fiber which protrudes from the baseplate and binds the eCIS to a target cell.

In some embodiments, the targeting domain comprises a heterologous binding moiety inserted/fused into the distal portion of the wildtype eCIS tail fiber protein. In some embodiments, the tail fiber comprises Pvc13.

In some embodiments, the tube comprises about 5, 10, 15, 20, 25, 30, 35, 40, 45, 50, 55, 60, 65, 70, 75, 80, 85, 90, 95 or 100 layers of a tube protein. In some embodiments, the sheath comprises about 5, 10, 15, 20, 25, 30, 35, 40, 45, 50, 55, 60, 65, 70, 75, 80, 85, 90, 95 or 100 layers of a sheath protein.

In some embodiments, the tube protein comprises the Pvc1 protein of Photorhabdus. In some embodiments, the tube protein comprises an amino acid sequence having at least about 85%, 86%, 87%, 88%, 89%, 90%, 91%, 92%, 93%, 94%, 95%, 96%, 97%, 98%, 99% or 100% sequence identity to SEQ ID NO: 1, or between about 80% to 100% sequence identity to SEQ ID NO: 1, or the tube protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 2, or between about 80% to 100% sequence identity to SEQ ID NO: 2.

In some embodiments, the plurality of tube connector proteins comprises the Pvc5 and/or Pvc7 protein of Photorhabdus. In some embodiments, the plurality of tube connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 3 and/or 5, or between about 80% to 100% sequence identity to SEQ ID NOs: 3 and/or 5, or the plurality of tube connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 4 and/or 6, or between about 80% to 100% sequence identity to SEQ ID NOs: 4 and/or 6.

In some embodiments, the sheath protein comprises the Pvc2 protein of Photorhabdus. In some embodiments, the sheath protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 7, or between about 80% to 100% sequence identity to SEQ ID NO: 7, or the sheath protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 8, or between about 80% to 100% sequence identity to SEQ ID NO: 8.

In some embodiments, the plurality of sheath connector proteins comprises the Pvc3 protein and/or Pvc4 protein of Photorhabdus. In some embodiments, the plurality of sheath connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 9 and/or 11, or between about 80% to 100% sequence identity to SEQ ID NOs: 9 and/or 11, or the plurality of sheath connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 10 and/or 12, or between about 80% to 100% sequence identity to SEQ ID NOs: 10 and/or 12.

In some embodiments, the plurality of baseplate proteins comprise the Pvc11 and/or Pvc12 proteins of Photorhabdus. In some embodiments, the plurality of baseplate proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 13 and/or 15, or between about 80% to 100% sequence identity to SEQ ID NOs: 13 and/or 15, or the plurality of baseplate proteins is encoded by a nucleic acid sequence having at least about 85%, 86%, 87%, 88%, 89%, 90%, 91%, 92%, 93%, 94%, 95%, 96%, 97%, 98%, 99% and/or 100% sequence identity to SEQ ID NOs: 14 and/or 16, or between about 80% to 100% sequence identity to SEQ ID NOs: 14 and/or 16.

In some embodiments, the plurality of spike proteins comprise the Pvc8 and/or Pvc10 proteins of Photorhabdus. In some embodiments, the plurality of spike proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 19 and/or 21, or between about 80% to 100% sequence identity to SEQ ID NOs: 19 and/or 21, or the plurality of spike proteins is encoded by a nucleic acid sequence having at least about 85%, 86%, 87%, 88%, 89%, 90%, 91%, 92%, 93%, 94%, 95%, 96%, 97%, 98%, 99% and/or 100% sequence identity to SEQ ID NOs: 20 and/or 22, or between about 80% to 100% sequence identity to SEQ ID NOs: 20 and/or 22.

In some embodiments, the terminal cap protein comprises the Pvc16 protein of Photorhabdus. In some embodiments, the terminal cap protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO:23, or between about 80% to 100% sequence identity to SEQ ID NO:23, or the terminal cap protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 24, or between about 80% to 100% sequence identity to SEQ ID NO: 24.

In some embodiments, the tail fiber protein comprises the Pvc13 protein of Photorhabdus. In some embodiments, the targeting domain comprises a tail fiber protein fused, linked or attached to a heterologous binding moiety, wherein the tail fiber comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 17, or between about 80% to 100% sequence identity to SEQ ID NO: 17, or the tail fiber is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 18, or between about 80% to 100% sequence identity to SEQ ID NO: 18.

In some embodiments, the heterologous binding moiety is selected from the group consisting of an antibody, an antibody fragment, a viral receptor-binding domain, an artificial receptor, a protein tag, and a tail fiber from an orthologous eCIS. In some embodiments, the antigen binding fragment is selected from the group consisting of a Fab, a Fab′, a F(ab′)2, an Fd, an Fv, a domain antibody (dAb), a complementarity determining region (CDR), a single chain variable fragment antibody (scFv), a maxibody, a minibody, an intrabody, a diabody, a triabody, a tetrabody, a v-NAR and a bis-scFvs. In some embodiments, the dAb is a shark antibody or a camelid antibody.

In some embodiments, the heterologous binding moiety is linked or attached to the tail fiber protein via a tag. In some embodiments, the tag is selected from the group consisting of a SNAP tag, a biotin tag, an Isopeptag, a SpyTag, a SpyCatcher tag, a SnoopTag, a SnoopTagJr, a SnoopCatcher tag, a DogTag, a DogCatcher tag, a Gluthatione-S-transferase tag, a CLIP tag, a Protein A tag, a Protein G tag, a Protein AG tag, a GFP tag, an HA tag, a FLAG tag, a Myc tag, a MoonTag, a SunTag, an Alfa tag, an EE tag, a His tag, and a HiBiT-tag.

In some embodiments, the eukaryotic cell of interest is a yeast cell, an insect cell, a mammalian cell, a plant cell or a fungi cell. In some embodiments, the mammalian cell is a human cell, a mouse cell, a rat cell, a cat cell, a dog cell, a horse cell, a sheep cell, a cow cell or a pig cell. In some embodiments, the human cell is a cancerous cell. In some embodiments, the eukaryotic cell of interest is part of a tissue, an organ or a whole organism. [0103) In some embodiments, the payload comprises an N-terminal packaging domain that is between 38 and 67 amino acids long. In some embodiments, the payload comprises an N-terminal packaging domain that is 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 amino acids long. In some embodiments, the N-terminal packaging domain comprises the first (i.e., from the N-terminal) 38, 39, 40, 41, 42, 43, 44, 45, 46, 47, 48, 49, 50, 51, 52, 53, 54, 55, 56, 57, 58, 59, 60, 61, 62, 63, 64, 65, 66, or 67 amino acids of SEQ ID NO: 25.

In some embodiments, the N-terminal packaging domain comprises N-terminal domain (NTD) of the SepC payload as shown in SEQ ID NO: 25. In some embodiments, the N-terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 25, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 26, or between about 80% to 100% sequence identity to SEQ ID NO: 26.

In some embodiments, the N-terminal packaging domain comprises N-terminal domain (NTD) of the Pnf payload as shown in SEQ ID NO: 27. In some embodiments, the N-terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 27, or the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 28, or between about 80% to 100% sequence identity to SEQ ID NO: 28.

In some embodiments, the protein payload is an arbitrary protein (any protein of interest). In some embodiments, the protein payload is between 5 and 2000, between 10 and 1000, between 100 and 500, between 500 and 1000, or between 1000 and 1500 and 1000 amino acids long. In some embodiments, the protein payload is about 5, 10, 20, 30, 40, 50, 75, 100, 150, 200, 250, 300, 350, 400, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1850, 1900, 1950, or 2000 amino acids long. In some embodiments, the protein payload is an enzyme, a cytokine or a binding agent (e.g., an antibody fragment, a Fab, a Fab′, a F(ab′)2, an Fd, an Fv, a domain antibody (dAb), a complementarity determining region (CDR), or a single chain variable fragment antibody (scFv)). In some embodiments, the protein payload is a gene editor, a chemotherapy agent or a toxin. In some embodiments, the gene editor is a Cre protein, a Cas protein, a Transcription activator-like effector nuclease (TALEN) or a Zing finger nuclease (ZFN). In some embodiments, the protein payload is a ribonucleoprotein (RNP). In a specific embodiment, the protein payload is a Cas RNP. In some embodiments, the eCIS system with engineered tropism is used to package and deliver a CRISPR-Cas protein or a fusion protein thereof (e.g., a base editor or prime editor). In some embodiments, the eCIS system with engineered tropism is used to package and deliver an RNP comprising a CRISPR-Cas protein or a fusion protein thereof (e.g., a base editor or prime editor), and a gRNA of the CRISPR-Cas protein.

In some embodiments, the protein payload or cargo protein is fused at its N-terminus to a packaging domain via a linker. In some embodiments, the protein payload or cargo protein is fused at its C-terminus to a packaging domain via a linker. In some embodiments, the linker is a cleavable linker. In some embodiments, cleavage of the linker in the target cell exposes a nuclear localization signal (NLS) positioned between the linker and the protein payload or cargo protein.

In some embodiments, the CRISPR-Cas protein or fusion protein thereof is fused at its N-terminus to a packaging domain via a linker. In some embodiments, the CRISPR-Cas protein or fusion protein thereof is fused at its C-terminus to a packaging domain via a linker. In some embodiments, the linker is a cleavable linker. In some embodiments, cleavage of the linker in the target cell exposes a nuclear localization signal (NLS) positioned between the linker and the CRISPR-Cas protein or fusion protein.

In some embodiments, there is a 3×GGSGG linker as shown in SEQ ID NO: 29 as a spacer between the N-terminal packaging domain and the protein payload or cargo protein.

In some embodiments, the payload is loaded into the tubular structure of the structural domain by an ATPase loader domain, thereby producing an assembled and loaded eCIS. In some embodiments, the ATPase loader domain comprises the Pvc15 protein of Photorhabdus. In some embodiments, the ATPase loader domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 31, or between about 80% to 100% sequence identity to SEQ ID NO: 31, or the ATPase loader domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 32, or between about 80% to 100% sequence identity to SEQ ID NO: 32.

In some embodiments, the eCIS is derived from Photorhabdus asymbiotica virulence cassette (PVC), Serratia entomophila anti-feeding prophage (AFP), Pseudoalteromonas luteoviolacea metamorphosis-associated contractile structure (MAC), Amoebophilus asiaticus T6SSiv, Pseudomonas aeruginosa T6SS, or Pseudomonas aeruginosa R-type bacteriocin. In some embodiments, the eCIS is derived from a Subtype Ia eCIS locus, a Subtype Ib eCIS locus, a Subtype IIa eCIS locus, a Subtype IIb eCIS locus, a Subtype IIc eCIS locus, or a Subtype IId eCIS locus.

In some embodiments, the protein payload is encoded by a nucleic acid in a separate vector than the vector encoding the structural domain, the targeting domain and the ATPAse loader domain of the eCIS. In some embodiments, the protein payload is encoded by a nucleic acid in the same vector as the vector encoding the structural domain, the targeting domain and the ATPAse loader domain of the eCIS.

### Expanded Example Payload Molecules

The engineered eCIS described herein may be used and further comprise a number of different payload (also known as “cargo”) for delivery. Representative payload may include, but are not limited to, nucleic acids, polynucleotides, proteins, polypeptides, polynucleotide/polypeptide complexes, small molecules, sugars, or a combination thereof.

Payloads that can be delivered in accordance with the systems and methods described herein include, but are not necessarily limited to, biologically active agents, including, but not limited to, therapeutic agents, imaging agents, and monitoring agents. A payload may be an exogenous material or an endogenous material.

### Biologically Active Agents

In some embodiments, the payload is a biologically active agent. Biologically active agents include any molecule that induces, directly or indirectly, an effect in a cell. Biologically active agents may be a protein, a nucleic acid, a small molecule, a carbohydrate, and a lipid. When the payload is or comprises a nucleic acid, the nucleic acid may be a separate entity from the DNA-based carrier. In these embodiments, the DNA-based carrier is not itself the payload. In other embodiments, the DNA-based carrier may itself comprise a nucleic acid payload. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein and the like. Reporter probes include photo-emitting compounds, such as phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors and regulator proteins used as recognition elements, which can be used either in whole cells or isolated and used independently (D'Souza, 2001, Biosensors and Bioelectronics 16:337-353).

One or two or more different payloads may be delivered by the delivery particles described herein.

In some embodiments, the payload may be linked to one or more sheath proteins by a linker, as described elsewhere herein. A suitable linker may include, but is not necessarily limited to, a glycine-serine linker. In some embodiments, the glycine-serine linker is (GGS)₃ (SEQ ID NO: 76).

In some embodiments, the payload comprises a ribonucleoprotein. In specific embodiments, the payload comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar terms. As used herein, “modulating” or “to modulate” generally means either reducing or inhibiting the activity of a target or antigen, or alternatively increasing the activity of the target or antigen, as measured using a suitable in vitro, cellular or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Gene Modifying Systems

In some embodiments, the payload is a polynucleotide modifying system or component(s) thereof. In some embodiments the polynucleotide modifying system is a gene modifying system. In some embodiments, the gene modifying system is or is composed of a gene modulating agent. In some embodiments, the genetic modulating agent may comprise one or more components of a polynucleotide modification system (e.g., a gene editing system) and/or polynucleotides encoding thereof.

In some embodiments, the gene editing system may be an RNA-guided system or other programmable nuclease system. In some embodiments, the gene editing system is an IscB system. In some embodiments, the gene editing system may be a CRISPR-Cas system.

### CRISPR-Cas Systems

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

### Class 1 Systems

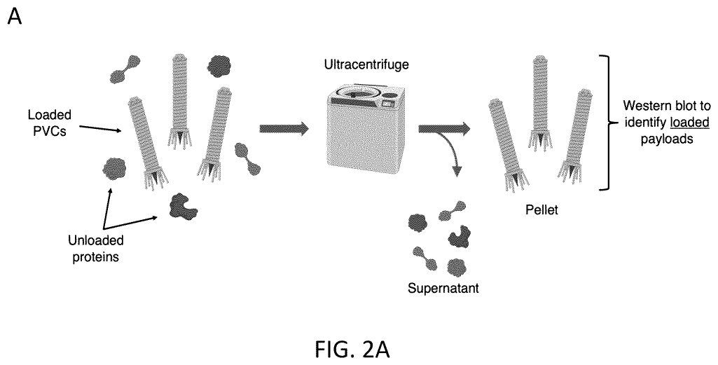

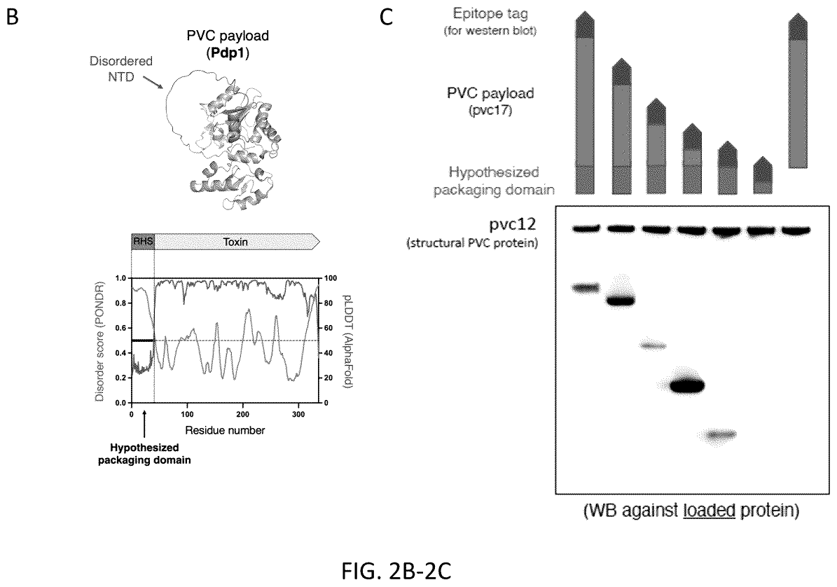

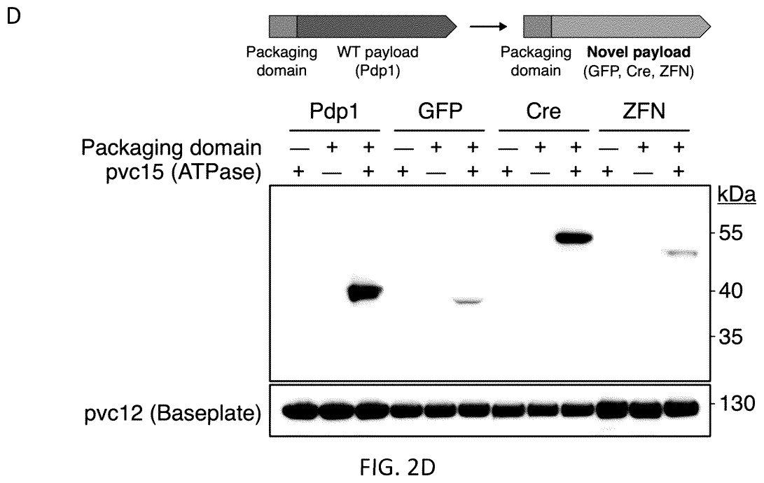

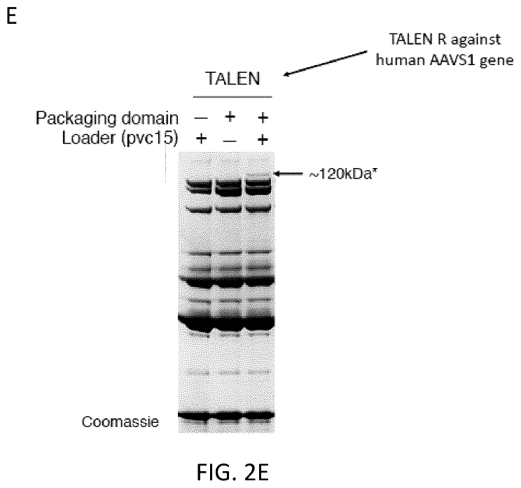

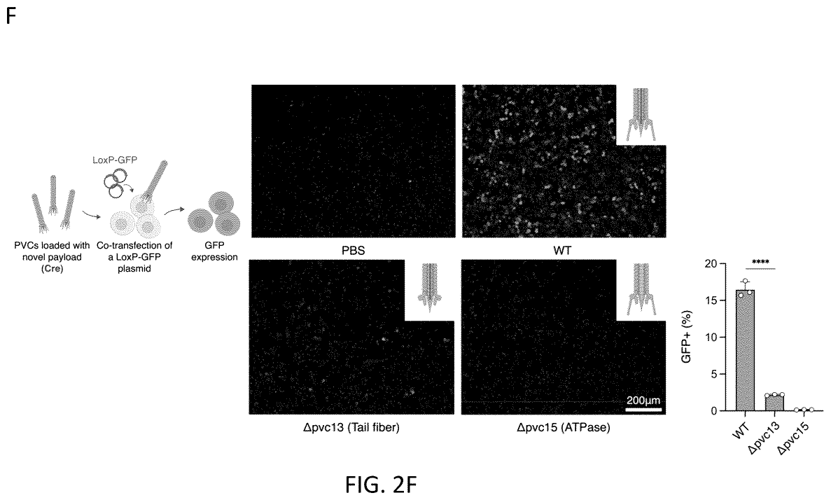

The methods, systems, and tools provided herein may be designed for use with Class 1 CRISPR proteins. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g., Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### Class 2 Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, CA), SOAP (available at soap. genomics. org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some embodiments, the target sequence may be a sequence within an RNA molecule selected from the group consisting of mRNA, pre-mRNA, and rRNA. In some embodiments, the target sequence may be a sequence within an RNA molecule selected from the group consisting of ncRNA, and lncRNA. In some embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm.

Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nucleotides (nt). In certain embodiments, the spacer length of the guide RNA is at least 15 nt. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333], which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA.

In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a specific embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 33) or PKKKRKVEAS (SEQ ID NO: 34); the NLS from nucleoplasmin (e.g., the nucleoplasmin bipartite NLS with the sequence KRPAATKKAGQAKKKK (SEQ ID NO: 35); the c-myc NLS having the amino acid sequence PAAKRVKLD (SEQ ID NO: 36) or RQRRNELKRSP (SEQ ID NO: 37); the hRNPA1 M9 NLS having the sequence NQSSNFGPMKGGNFGGRSSGPYGGGGQYFAKPRNQGGY (SEQ ID NO: 38); the sequence RMRIZFKNKGKDTAELRRRRVEVSVELRKAKKDEQILKRRNV (SEQ ID NO: 39) of the IBB domain from importin-alpha; the sequences VSRKRPRP (SEQ ID NO: 40) and PPKKARED (SEQ ID NO: 67) of the myoma T protein; the sequence PQPKKKPL (SEQ ID NO: 41) of human p53; the sequence SALIKKKKKMAP (SEQ ID NO: 42) of mouse c-abl IV; the sequences DRLRR (SEQ ID NO: 43) and PKQKKRK (SEQ ID NO: 44) of the influenza virus NS1; the sequence RKLKKKIKKL (SEQ ID NO: 45) of the Hepatitis virus delta antigen; the sequence REKKKFLKRR (SEQ ID NO: 46) of the mouse Mx1 protein; the sequence KRKGDEVDGVDEVAKKKSKK (SEQ ID NO: 47) of the human poly(ADP-ribose) polymerase; and the sequence RKCLQAGMNLEARKTKK (SEQ ID NO: 48) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In some embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein.

In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

It will be appreciated that NLS and NES described herein with respect to Cas proteins can be used with other payloads, in particularly, gene modifying agents herein, and other proteins that can benefit from translocation in or out of a nuclease of a cell, such as a target cell.

### Donor Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides).

In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

Split CRISPR-Cas systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference. 10.1861 In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide payload that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

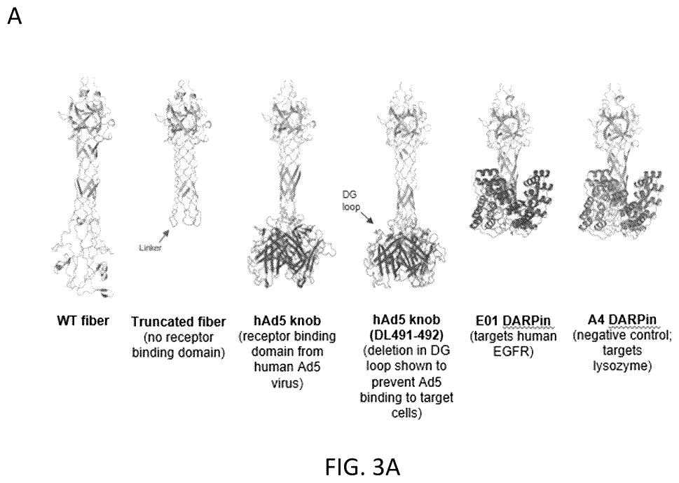

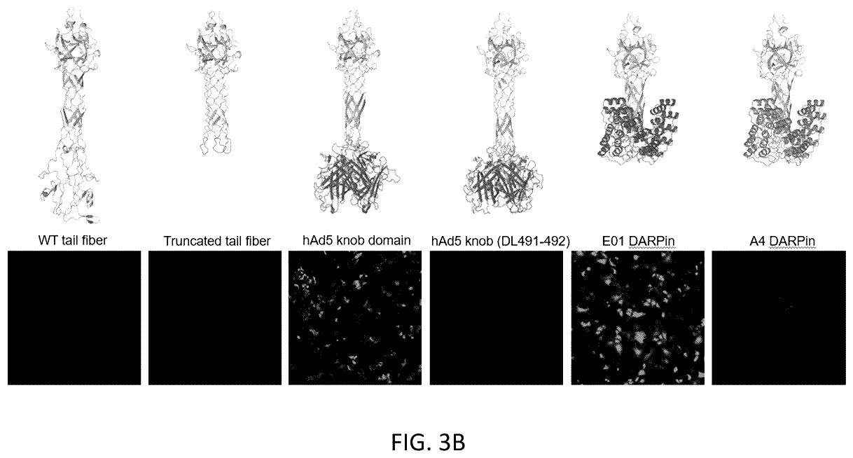

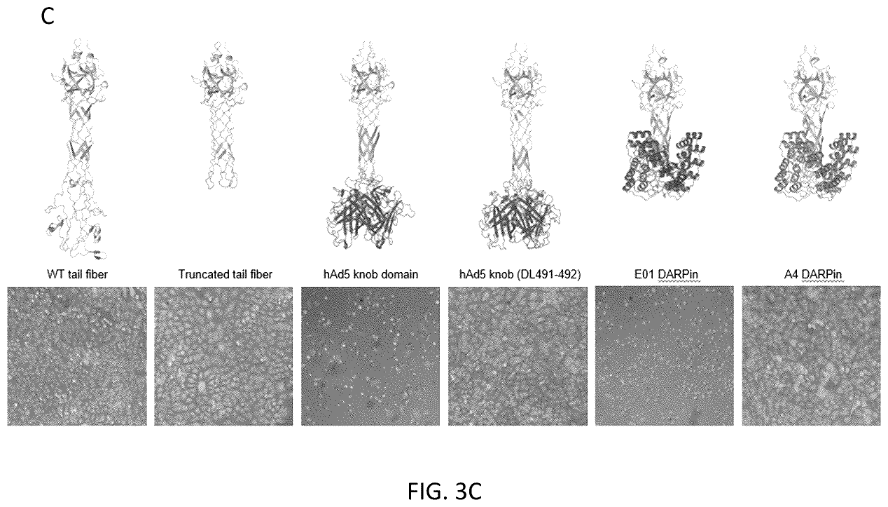

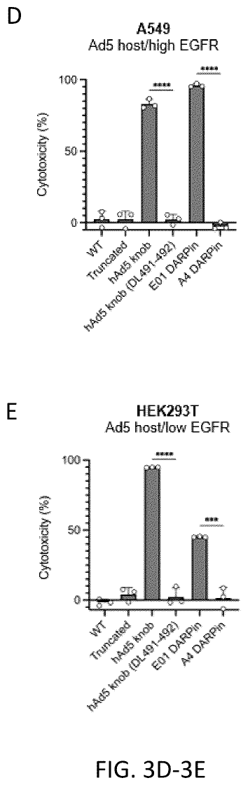

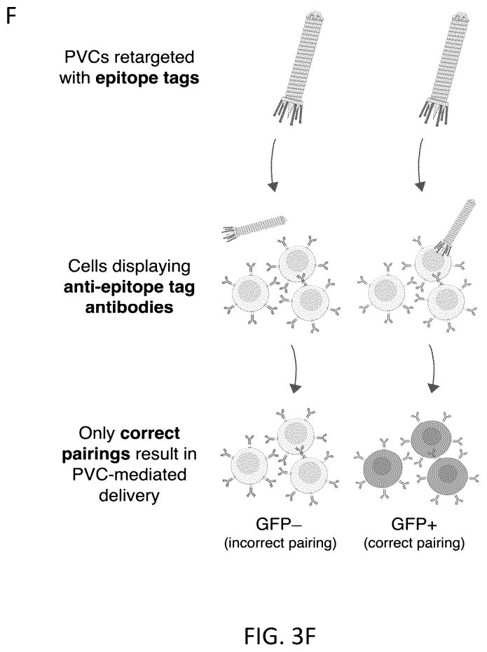

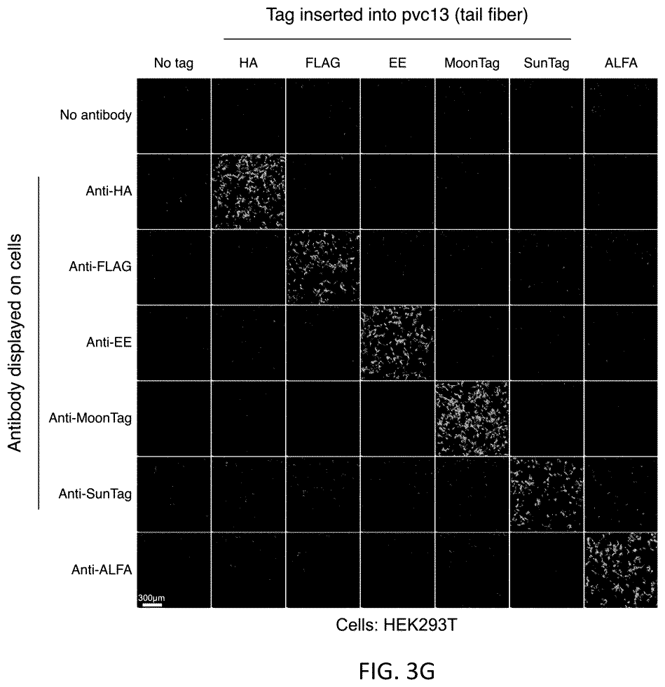

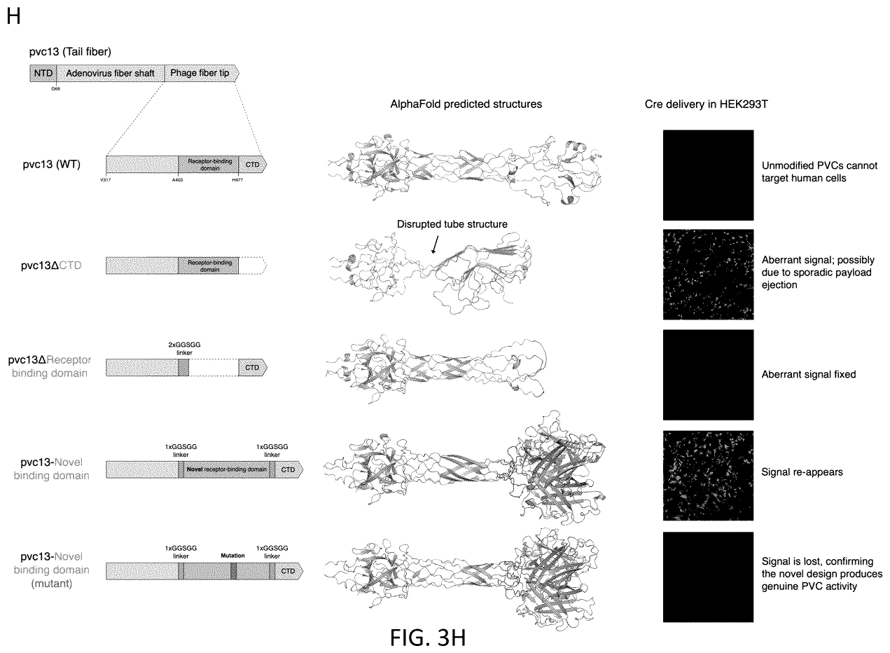

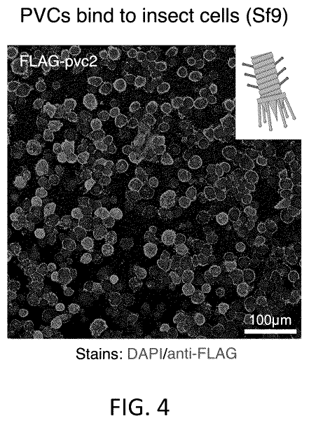

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### IscBs

In some embodiments, the nucleic acid-guided nucleases herein may be IscB proteins. An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated.

In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the IscBs may comprise one or more domains, e.g., one or more of a X domain (e.g., at N-terminus), a RuvC domain, a Bridge Helix domain, and a Y domain (e.g., at C-terminus). In some examples, the nucleic-acid guided nuclease comprises an N-terminal X domain, a RuvC domain (e.g., including a RuvC-I, RuvC-II, and RuvC-III subdomains), a Bridge Helix domain, and a C-terminal Y domain. In some examples, the nucleic-acid guided nuclease comprises an N-terminal X domain, a RuvC domain (e.g., including a RuvC-I, RuvC-II, and RuvC-III subdomains), a Bridge Helix domain, an HNH domain, and a C-terminal Y domain.

In some embodiments, the nucleic acid-guided nucleases may have a small size. For example, the nucleic acid-guided nucleases may be no more than 50, no more than 100, no more than 150, no more than 200, no more than 250, no more than 300, no more than 350, no more than 400, no more than 450, no more than 500, no more than 550, no more than 600, no more than 650, no more than 700, no more than 750, no more than 800, no more than 850, no more than 900, no more than 950, or no more than 1000 amino acids in length.

In some examples, the IscB protein shares at least 8000, at least 85% o, at least 90%, at least 95%, at least 99%, or 100% o sequence identity with a IscB protein selected from Table 2.

TABLE 2

| No. | Proteins | Sequences |
|---|---|---|
| 1 | IscB(−HNH) | MSTDATLIRTTPSHAEADATDTLVATPLMPPRRVISPWPGPGE |
| | EFH81386 | GQSLMRIPVVDIRGMALMPCTPAKARHLLKSGNARPKRNKL |
| | | GLFYVQLSYEQEPDNQSLVAGVDPGSKFEGLSVVGTKDTVL |
| | | NLMVEAPDHVKGAVQTRRTMRRARRQRKWRRPKRFHNRLN |
| | | RMQRIPPSTRSRWEAKARIVAHLRTILPFTDVVVEDVQAVTR |
| | | KGKGGTWNGSFSPVQVGKEHLYRLLRAMGLTLHLREGWQT |
| | | KELREQHGLKKTKSKSKQSFESHAVDSWVLAASISGAEHPTC |
| | | TRLWYMVPAILHRRQLHRLQASKGGVRKPYGGTRSLGVKRG |
| | | TLVEHKKYGRCTVGGVDRKRNTISLHEYRTNTRLTQAAKVE |
| | | TCRVLTWLSWRSWLLRGKRTSSKGKGSHSS (SEQ ID NO: 49) |
| 2 | IscB(+HNH) | MQPAKQQNWVFQINGDKQPLDMINPGRCRELQNRGKLASFR |
| | TAE54104.1 | RFPYVVIQQQTIENPQTKEYILKIDPGSQWTGFAIQCGNDILFR |
| | | AELNHRGEAIKFDLVKRAWFRRGRRSRNLRYRKKRLNRAKP |
| | | EGWLAPSIRHRVLTVETWIKRFMRYCPIAWIEIEQVRFDTQKL |
| | | ANPEIDGVEYQQGELQGYEVREYLLQKWGRKCAYCGTENVP |
| | | LEVEHIQSKSKGGSSRIGNLTLACHVCNVKKGNLDVRDFLAK |
| | | SPDILNQVLENSTKPLKDAAAVNSTRYAIVKMAKSICENVKC |
| | | SSGARTKMNRVRQGLEKTHSLDAACVGESGASIRVLTDRPLL |
| | | ITCKGHGSRQSIRVNASGFPAVKNAKTVFTHIAAGDVVRFTIG |
| | | KDRKKAQAGTYTARVKTPTPKGFEVLIDGAR |
| | | ISLSTMSNVVFVHRSDGYGYEL (SEQ ID NO: 50) |
| 3 | IscB(+HNH) | MAVFVIDKHKRPLMPCSEKRARLLLERGRAVVHRQVPFV |
| | WP_038093640.1 | IRLKDRTVQHSAVQPLRVALDPGSRATGMALVREKNTVD |
| | | TGTGEVYRERIALNLFELVHRGHRIREQLDQRRNFRRRRR |
| | | GANLRYRAPRFDNRRRPPGWLAPSLQHRVDTTMAWVRR |
| | | LCRWAPASAIGIETVRFDTQRLQNPEISGVEYQQGALAGC |
| | | EVREYLLEKWGRKCAYCGAENVPLEIEHIVPKSRGGSDRV |
| | | SNLALACRACNQAKGNRDVRAFLADQPERLARILAQAKA |
| | | PLKDAAAVNATRWALYRALVDTGLPVEAGTGGRTKWNR |
| | | TRLGLPKTHALDALCVGQVDQVRHWRVPVLGIRCAGRGS |
| | | YRRTRLTRHGFPRGYLTRNKSAFGFQTGDLIRAVVTKGK |
| | | KAGTYLGRIAIRASGSFNIQTPMGVVQGIHHRFCTLLQRA |
| | | DGYGYFVQPKPTEAALSSPRLKAGVSSAGN (SEQ ID NO: |
| | | 51) |
| 4 | IscB(+HNH) | MTTNVVFVIDTNQKPLQPCSAAVARKLLLRGKAAMFRRY |
| | WP_052490348.1 | PAVIILKKEVDSVGKPKIELRIDPGSKYTGFALDSKDNAD |
| | | FIIWGTELEHRGAAICKELTKRSAIRRSRRNRKTRYRKKRF |
| | | ERRKPEGWLAPSLQHRVDTTLTWVKRICKFVPIMSISVEQ |
| | | VKFDLQKLENSDIQGIEYQQGTLAGYTLREALLEHWGRK |
| | | CAYCDVENVFLEIEHIYPKSKGGSDKFSNLTLACHKCNIN |
| | | KGNKSIDEFLLSDHKRLEQIKLHQKKTLKDAAAVNATRK |
| | | KLVTTLQEKTFLNVLVSDGASTKMTRLSSSLAKRHWIDA |
| | | GCVNTTLIVILKTLQPLQVKCNGHGNKQFVTMDAYGFPR |
| | | KSYEPKKVRKDWKAGDIIRVTKKDGTMLMGRVKKAAKK |
| | | LVYIPFGGKEASFSSENAKAIHRSDGYRYSFAAIDSELLQK |
| | | MAT (SEQ ID NO: 52) |
| 5 | IscB(+HNH) | MPNKYAFVLDSKGKLLDPTKSKKAWYLIRKGKASLVEEY |
| | WP_015325818.1 | PLIIKLKREVPKDQVNSDKLILGIDDGTKKVGFALVQKCQ |
| | | TKNKVLFKAVMEQRQDVSKKMEERRGYRRYRRSHKRYR |
| | | PARFDNRSSSKRKGRIPPSILQKKQAILRVVNKLKKYIRID |
| | | KIVLEDVSIDIRKLTEGRELYNWEYQESNRLDENLRKATL |
| | | YRDDCTCQLCGTTETMLHAHHIMPRRDGGADSIYNLITLC |
| | | KACHKDKVDNNEYQYKDQFLAIIDSKELSDLKSASHVMQ |
| | | GKTWLRDKLSKIAQLEITSGGNTANKRIDYEIEKSHSNDAI |
| | | CTTGLLPVDNIDDIKEYYIKPLRKKSKAKIKELKCFRQRDL |
| | | VKYTKRNGETYTGYITSLRIKNNKYNSKVCNFSTLKGKIF |
| | | RGYGFRNLTLLNRPKGLMIV (SEQ ID NO: 53) |
| 6 | sp\|G3ECR1\| | MLFNKCIIISINLDFSNKEKCMTKPYSIGLDIGTNSVGWAVI |
| | CAS9_STRTR | TDNYKVPSKKMKVLGNTSKKYIKKNLLGVLLFDSGITAE |
| | | GRRLKRTARRRYTRRRNRILYLQEIFSTEMATLDDAFFQR |
| | | LDDSFLVPDDKRDSKYPIFGNLVEEKVYHDEFPTIYHLRK |
| | | YLADSTKKADLRLVYLALAHMIKYRGHFLIEGEFNSKNN |
| | | DIQKNFQDFLDTYNAIFESDLSLENSKQLEEIVKDKISKLE |
| | | KKDRILKLFPGEKNSGIFSEFLKLIVGNQADFRKCFNLDEK |
| | | ASLHFSKESYDEDLETLLGYIGDDYSDVFLKAKKLYDAIL |
| | | LSGFLTVTDNETEAPLSSAMIKRYNEHKEDLALLKEYIRNI |
| | | SLKTYNEVFKDDTKNGYAGYIDGKTNQEDFYVYLKNLLA |
| | | EFEGADYFLEKIDREDFLRKQRTFDNGSIPYQIHLQEMRAI |
| | | LDKQAKFYPFLAKNKERIEKILTFRIPYYVGPLARGNSDFA |
| | | WSIRKRNEKITPWNFEDVIDKESSAEAFINRMTSFDLYLPE |
| | | EKVLPKHSLLYETFNVYNELTKVRFIAESMRDYQFLDSKQ |
| | | KKDIVRLYFKDKRKVTDKDIIEYLHAIYGYDGIELKGIEKQ |
| | | FNSSLSTYHDLLNIINDKEFLDDSSNEAIIEEIIHTLTIFEDRE |
| | | MIKQRLSKFENIFDKSVLKKLSRRHYTGWGKLSAKLINGI |
| | | RDEKSGNTILDYLIDDGISNRNFMQLIHDDALSFKKKIQKA |
| | | QIIGDEDKGNIKEVVKSLPGSPAIKKGILQSIKIVDELVKVM |
| | | GGRKPESIVVEMARENQYTNQGKSNSQQRLKRLEKSLKE |
| | | LGSKILKENIPAKLSKIDNNALQNDRLYLYYLQNGKDMYT |
| | | GDDLDIDRLSNYDIDHIIPQAFLKDNSIDNKVLVSSASNRG |
| | | KSDDFPSLEVVKKRKTFWYQLLKSKLISQRKFDNLTKAER |
| | | GGLLPEDKAGFIQRQLVETRQITKHVARLLDEKENNKKDE |
| | | NNRAVRTVKIITLKSTLVSQFRKDFELYKVREINDFHHAH |
| | | DAYLNAVIASALLKKYPKLEPEFVYGDYPKYNSFRERKSA |
| | | TEKVYFYSNIMNIFKKSISLADGRVIERPLIEVNEETGESV |
| | | WNKESDLATVRRVLSYPQVNVVKKVEEQNHGLDRGKPK |
| | | GLFNANLSSKPKPNSNENLVGAKEYLDPKKYGGYAGISNS |
| | | FAVLVKGTIEKGAKKKITNVLEFQGISILDRINYRKDKLNF |
| | | LLEKGYKDIELIIELPKYSLFELSDGSRRMLASILSTNNKRG |
| | | EIHKGNQIFLSQKFVKLLYHAKRISNTINENHRKYVENHK |
| | | KEFEELFYYILEFNENYVGAKKNGKLLNSAFQSWQNHSID |
| | | ELCSSFIGPTGSERKGLFELTSRGSAADFEFLGVKIPRYRDY |
| | | TPSSLLKDATLIHQSVTGLYETRIDLAKLGEG (SEQ ID NO: |
| | | 54) |
| 7 | sp\|J7RUA5\| | MKRNYILGLDIGITSVGYGIIDYETRDVIDAGVRLFKEANV |
| | CAS9_STAAU | ENNEGRRSKRGARRLKRRRRHRIQRVKKLLFDYNLLTDH |
| | | SELSGINPYEARVKGLSQKLSEEEFSAALLHLAKRRGVHN |
| | | VNEVEEDTGNELSTKEQISRNSKALEEKYVAELQLERLKK |
| | | DGEVRGSINRFKTSDYVKEAKQLLKVQKAYHQLDQSFID |
| | | TYIDLLETRRTYYEGPGEGSPFGWKDIKEWYEMLMGHCT |
| | | YFPEELRSVKYAYNADLYNALNDLNNLVITRDENEKLEY |
| | | YEKFQIIENVFKQKKKPTLKQIAKEILVNEEDIKGYRVTST |
| | | GKPEFTNLKVYHDIKDITARKEIIENAELLDQIAKILTIYQS |
| | | SEDIQEELTNLNSELTQEEIEQISNLKGYTGTHNLSLKAINL |
| | | ILDELWHTNDNQIAIFNRLKLVPKKVDLSQQKEIPTTLVDD |
| | | FILSPVVKRSFIQSIKVINAIIKKYGLPNDIIIELAREKNSKDA |
| | | QKMINEMQKRNRQTNERIEEIIRTTGKENAKYLIEKIKLHD |
| | | MQEGKCLYSLEAIPLEDLLNNPFNYEVDHIIPRSVSFDNSF |
| | | NNKVLVKQEENSKKGNRTPFQYLSSSDSKISYETFKKHIL |
| | | NLAKGKGRISKTKKEYLLEERDINRFSVQKDFINRNLVDT |
| | | RYATRGLMNLLRSYFRVNNLDVKVKSINGGFTSFLRRKW |
| | | KFKKERNKGYKHHAEDALIIANADFIFKEWKKLDKAKKV |
| | | MENQMFEEKQAESMPEIETEQEYKEIFITPHQIKHIKDFKD |
| | | YKYSHRVDKKPNRELINDTLYSTRKDDKGNTLIVNNLNG |
| | | LYDKDNDKLKKLINKSPEKLLMYHHDPQTYQKLKLIMEQ |
| | | YGDEKNPLYKYYEETGNYLTKYSKKDNGPVIKKIKYYGN |
| | | KLNAHLDITDDYPNSRNKVVKLSLKPYRFDVYLDNGVYK |
| | | FVTVKNLDVIKKENYYEVNSKCYEEAKKLKKISNQAEFIA |
| | | SFYNNDLIKINGELYRVIGVNNDLLNRIEVNMIDITYREYL |
| | | ENMNDKRPPRIIKTIASKTQSIKKYSTDILGNLYEVKSKKH |
| | | PQIIKKG (SEQ ID NO: 55) |
| 8 | Streptococcus | KYSIGLDIGTNSVGWAVITDEYKVPSKKFKVLGNTDRHSI |
| | pyogenes_ | KKNLIGALLFDSGETAEATRLKRTARRRYTRRKNRICYLQ |
| | SF370 | EIFSNEMAKVDDSFFHRLEESFLVEEDKKHERHPIFGNIVD |
| | | EVAYHEKYPTIYHLRKKLVDSTDKADLRLIYLALAHMIKF |
| | | RGHFLIEGDLNPDNSDVDKLFIQLVQTYNQLFEENPINASG |
| | | VDAKAILSARLSKSRRLENLIAQLPGEKKNGLFGNLIALSL |
| | | GLTPNFKSNFDLAEDAKLQLSKDTYDDDLDNLLAQIGDQ |
| | | YADLFLAAKNLSDAILLSDILRVNTEITKAPLSASMIKRYD |
| | | EHHQDLTLLKALVRQQLPEKYKEIFFDQSKNGYAGYIDG |
| | | GASQEEFYKFIKPILEKMDGTEELLVKLNREDLLRKQRTF |
| | | DNGSIPHQIHLGELHAILRRQEDFYPFLKDNREKIEKILTFR |
| | | IPYYVGPLARGNSRFAWMTRKSEETITPWNFEEVVDKGAS |
| | | AQSFIERMTNFDKNLPNEKVLPKHSLLYEYFTVYNELTKV |
| | | KYVTEGMRKPAFLSGEQKKAIVDLLFKTNRKVTVKQLKE |
| | | DYFKKIECFDSVEISGVEDRFNASLGTYHDLLKIIKDKDFL |
| | | DNEENEDILEDIVLTLTLFEDREMIEERLKTYAHLFDDKV |
| | | MKQLKRRRYTGWGRLSRKLINGIRDKQSGKTILDFLKSDG |
| | | FANRNFMQLIHDDSLTFKEDIQKAQVSGQGDSLHEHIANL |
| | | AGSPAIKKGILQTVKVVDELVKVMGRHKPENIVIEMAREN |
| | | QTTQKGQKNSRERMKRIEEGIKELGSQILKEHPVENTQLQ |
| | | NEKLYLYYLQNGRDMYVDQELDINRLSDYDVDHIVPQSF |
| | | LKDDSIDNKVLTRSDKNRGKSDNVPSEEVVKKMKNYWR |
| | | QLLNAKLITQRKFDNLTKAERGGLSELDKAGFIKRQLVET |
| | | RQITKHVAQILDSRMNTKYDENDKLIREVKVITLKSKLVS |
| | | DFRKDFQFYKVREINNYHHAHDAYLNAVVGTALIKKYPK |
| | | LESEFVYGDYKVYDVRKMIAKSEQEIGKATAKYFFYSNIM |
| | | NFFKTEITLANGEIRKRPLIETNGETGEIVWDKGRDFATVR |
| | | KVLSMPQVNIVKKTEVQTGGFSKESILPKRNSDKLIARKK |
| | | DWDPKKYGGFDSPTVAYSVLVVAKVEKGKSKKLKSVKE |
| | | LLGITIMERSSFEKNPIDFLEAKGYKEVKKDLIIKLPKYSLF |
| | | ELENGRKRMLASAGELQKGNELALPSKYVNFLYLASHYE |
| | | KLKGSPEDNEQKQLFVEQHKHYLDEIIEQISEFSKRVILAD |
| | | ANLDKVLSAYNKHRDKPIREQAENIIHLFTLTNLGAPAAF |
| | | KYFDTTIDRKRYTSTKEVLDATLIHQSITGLYETRIDLSQL |
| | | GGD (SEQ ID NO: 56) |

TABLE 3

| | | Domains and amino |
|---|---|---|
| No. | Proteins | acid positions |
| 1 | IscB(−HNH) | X domain: 51-97 |
| | EFH81386 | RuvC-I: 104-118 |
| | | Bridge Helix: 140-160 |
| | | RuvC-II: 169-212 |
| | | RuvC-III: 226-278 |
| 2 | IscB(+HNH) | X domain: 11-56 |
| | TAE54104.1 | RuvC-I: 63-77 |
| | | Bridge Helix: 100-121 |
| | | RuvC-II: 129-172 |
| | | HNH: 211-243 |
| | | RuvC-III: 279-321 |
| 3 | IscB(+HNH) | X domain: 4-50 |
| | WP_038093640.1 | RuvC-I: 57-71 |
| | | Bridge Helix: 108-129 |
| | | RuvC-II: 138-181 |
| | | HNH: 220-252 |
| | | RuvC-III: 288-330 |
| 4 | IscB(+HNH) | X domain: 7-52 |
| | WP_052490348.1 | RuvC-I: 59-73 |
| | | Bridge Helix: 100-121 |
| | | RuvC-II: 129-172 |
| | | HNH: 211-243 |
| | | RuvC-III: 279-322 |
| 5 | IscB(+HNH) | X domain: 7-52 |
| | WP_015325818.1 | RuvC-I: 61-75 |
| | | Bridge Helix: 101-121 |
| | | RuvC-II: 132-175 |
| | | HNH: 215-247 |
| | | RuvC-III: 284-327 |
| 6 | sp\|G3ECR1\|CAS9_STRTR | RuvC-I: 28-42 |
| | | Bridge Helix: 85-108 |
| | | Rec: 118-736 |
| | | RuvC-II: 750-799 |
| | | HNH: 864-896 |
| | | RuvC-III: 957-1019 |
| | | PAM Interaction |
| | | (PI): 1119-1409 |
| 7 | sp\|J7RUA5\|CAS9_STAAU | RuvC-I: 7-21 |
| | | Bridge Helix: 49-72 |
| | | Rec: 80-433 |
| | | RuvC-II: 445-493 |
| | | HNH: 553-585 |
| | | RuvC-III: 654-709 |
| | | PAM Interaction |
| | | (PI): 789-1053 |
| 8 | Streptococcus_pyogenes_SF370 | RuvC-I: 4-18 |
| | | Bridge Helix: 61-84 |
| | | Rec: 94-718 |
| | | RuvC-II: 725-774 |
| | | HNH: 833-865 |
| | | RuvC-III: 926-988 |
| | | PAM Interaction |
| | | (PI): 1099-1365 |

### X Domains

In some embodiments, the IscB proteins comprise an X domain, e.g., at its N-terminal.

In certain embodiments, the X domain include the X domains in Table 3. Examples of the X domains also include any polypeptides a structural similarity and/or sequence similarity to a X domain described in the art. In some examples, the X domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with X domains in Table 3.

In some examples, the X domain may be no more than 10, no more than 20, no more than 30, no more than 40, no more than 50, no more than 60, no more than 70, no more than 80, no more than 90, or no more than 100 amino acids in length. For example, the X domain may be no more than 50 amino acids in length, such as comprising 2 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

### Y Domain

In some embodiments, the IscB proteins comprise a Y domain, e.g., at its C-terminal.

In certain embodiments, the X domain include Y domains in Table 3. Examples of the Y domain also include any polypeptides a structural similarity and/or sequence similarity to a Y domain described in the art. In some examples, the Y domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with Y domains in Table 3.

### RuvC Domain

In some embodiments, the IscB proteins comprises at least one nuclease domain. In certain embodiments, the IscB proteins comprise at least two nuclease domains. In certain embodiments, the one or more nuclease domains are only active upon presence of a cofactor. In certain embodiments, the cofactor is Magnesium (Mg). In embodiments where more than one nuclease domain is present and the substrate is a double-strand polynucleotide, the nuclease domains each cleave a different strand of the double-strand polynucleotide. In certain embodiments, the nuclease domain is a RuvC domain.

The IscB proteins may comprise a RuvC domain. The RuvC domain may comprise multiple subdomains, e.g., RuvC-I, RuvC-II and RuvC-III. The subdomains may be separated by interval sequences on the amino acid sequence of the protein.

In certain embodiments, examples of the RuvC domain include those in Table 3. Examples of the RuvC domain also include any polypeptides a structural similarity and/or sequence similarity to a RuvC domain described in the art. For example, the RuvC domain may share a structural similarity and/or sequence similarity to a RuvC of Cas9. In some examples, the RuvC domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with RuvC domains in Table 3.

### Bridge Helix

The IscB proteins comprise a bridge helix (BH) domain. The bridge helix domain refers to a helix and arginine rich polypeptide. The bridge helix domain may be located next to anyone of the amino acid domains in the nucleic-acid guided nuclease. In some embodiments, the bridge helix domain is next to a RuvC domain, e.g., next to RuvC-I, RuvC-II, or RuvC-III subdomain. In one example, the bridge helix domain is between a RuvC-1 and RuvC2 subdomains.

The bridge helix domain may be from 10 to 100, from 20 to 60, from 30 to 50, e.g., 35, 36, 37, 38, 39, 40, 41, 42, 43, 44, 45, 46 or 47, 48, 49, or 50 amino acids in length. Examples of bridge helix includes the polypeptide of amino acids 60-93 of the sequence of S. pyogenes Cas9.

In certain embodiments, examples of the BH domain include those in Table 3. Examples of the BH domain also include any polypeptides a structural similarity and/or sequence similarity to a BH domain described in the art. For example, the BH domain may share a structural similarity and/or sequence similarity to a BH domain of Cas9. In some examples, the BH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with BH domains in Table 3.

### HNH Domain

The IscB proteins comprise an HNH domain. In certain embodiments, at least one nuclease domain shares a substantial structural similarity or sequence similarity to a HNH domain described in the art.

In some examples, the nucleic acid-guided nuclease comprises a HNH domain and a RuvC domain. In the cases where the RuvC domain comprises RuvC-I, RuvC-II, and RuvC-III domain, the HNH domain may be located between the Ruv C II and RuvC III subdomains of the RuvC domain.

In certain embodiments, examples of the HNH domain include those in Table 3. Examples of the HNH domain also include any polypeptides a structural similarity and/or sequence similarity to a HNH domain described in the art. For example, the HNH domain may share a structural similarity and/or sequence similarity to a HNH domain of Cas9. In some examples, the HNH domain may have an amino acid sequence that share at least 50%, at least 55%, at least 60%, at least 5%, at least 70%, at least 75%, at least 80%, at least 85%, at least 90%, at least 95%, at least 99%, or 100% sequence identity with HNH domains in Table 3.

hRNA

In some examples, the IscB proteins capable of forming a complex with one or more hRNA molecules. The hRNA complex can comprise a guide sequence and a scaffold that interacts with the IscB polypeptide. An hRNA molecules may form a complex with an IscB polypeptide nuclease or IscB polypeptide and direct the complex to bind with a target sequence. In certain example embodiments, the hRNA molecule is a single molecule comprising a scaffold sequence and a spacer sequence. In certain example embodiments, the spacer is 5′ of the scaffold sequence. In certain example embodiments, the hRNA molecule may further comprise a conserved nucleic acid sequence between the scaffold and spacer portions.

As used herein, a heterologous hRNA molecule is an hRNA molecule that is not derived from the same species as the IscB polypeptide nuclease, or comprises a portion of the molecule, e.g., spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein, the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 58)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

An exemplary amino acid sequence of a C-terminal capping region is:

(SEQ ID NO: 57)
M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

As used herein, the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full-length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Polypeptides

In certain example embodiments, the payload molecule may one or more polypeptides. The polypeptide may be a full-length protein or a functional fragment or functional domain thereof, that is a fragment or domain that maintains the desired functionality of the full-length protein. As used within this section, “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Secretory Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In some embodiments, the protein is secreted by the secretory pathway. In some embodiments, the proteins are exocrine secretion proteins or peptides, comprising enzymes in the digestive tract. In some embodiments the protein is endocrine secretion protein or peptide, for example, insulin and other hormones released into the blood stream. In some embodiments, the protein is involved in signaling between or within cells via secreted signaling molecules, for example, paracrine, autocrine, endocrine or neuroendocrine. In some embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a specific embodiment, the one or more polynucleotides is inserted in the middle of the c-peptide. In some aspects, the secretory protein is GLP-1, glucagon, betatrophin, pancreatic amylase, pancreatic lipase, carboxypeptidase, secretin, CCK, a PPAR (e.g. PPAR-alpha, PPAR-gamma, PPAR-delta or a precursor thereof (e.g. preprotein or preproprotein). In aspects, the secretory protein is fibronectin, a clotting factor protein (e.g. Factor VII, VIII, IX, etc.), α2-macroglobulin, al-antitrypsin, antithrombin III, protein S, protein C, plasminogen, α2-antiplasmin, complement components (e.g. complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+response or a CD8+response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+thymocytes, CD4−/CD8−thymocytes, 76 T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins may be immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-P, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-α, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR), or may include one or more PPARs. In some embodiments, the protein is PPARα, PPAR 7 is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survival Functions

In certain example embodiments, the one or more polypeptides may comprise an proteins that proteins that promote tissue regeneration and/or transplant survival functions. In some cases, such proteins may induce and/or up-regulate the expression of genes for pancreatic R cell regeneration. In some cases, the proteins that promote transplant survival and functions include the products of genes for pancreatic p cell regeneration. Such genes may include proislet peptides that are proteins or peptides derived from such proteins that stimulate islet cell neogenesis. Examples of genes for pancreatic R cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfa1, Mafa, Pax6, Pax4, Nkx6.1, Nkx2.2, PDGF, vglycin, placental lactogens (somatomammotropins, e.g. CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or more polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoidds. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is a-defensin HD-6, HNP-1 and β-defensin hBD-3, lysozyme, cathelcidin LL-37, C-type lectin RegIIIalpha, for example. See, e.g. Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some aspects, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed with. In some aspects, the anti-fibrillating agent is P4 (VITYF) (SEQ ID NO: 59), P5 (VVVVV) (SEQ ID NO: 60), KR7 (KPWWPRR) (SEQ ID NO: 61), NK9 (NIVNVSLVK) (SEQ ID NO: 62), iAb5p (Leu-Pro-Phe-Phe-Asp) (SEQ ID NO: 63), KLVF (SEQ ID NO: 64) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA) (SEQ ID NO: 77), RI-OR2-TAT, cyclo(17, 21)-(Lys17, Asp21)A_(1-28), SEN304, SEN1576, D3, R8-Aβ(25-35), human yD-crystallin (HGD), poly-lysine, heparin, poly-Asp, polyGl, poly-L-lysine, poly-L-glutamic acid, LVEALYL (SEQ ID NO: 65), RGFFYT (SEQ ID NO: 66), a peptide set forth or as designed/generated by the method set forth in U.S. Pat. No. 8,754,034, and combinations thereof. In some embodiments, the anti-fibrillating agent is a D-peptide. In some embodiments, the anti-fibrillating agent is an L-peptide. In some embodiments, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In some embodiments, the retro-inverso modified peptide is derived from a natural or synthetic A3 peptide. In some aspects, the polynucleotide encodes a fibrillation resistant protein. In some aspects, the fibrillation resistant protein is a modified insulin, see e.g. U.S. Pat. No. 8,343,914.

### Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such, these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by R pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 PM. Antibodies with affinities greater than 1×107 M-1 (or a dissociation coefficient of 1 M or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Protease Cleavage Sites

The one or more payload polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1 (ULP-1) protease cleavage site. In certain examples, the protease cleavage site comprises Lys-Arg.

### Engineered Cells

Described herein are various aspects of engineered cells that can include one or more of the engineered delivery system polynucleotides, polypeptides, vectors, and/or vector systems, and/or engineered delivery vesicles (e.g., those produced from an engineered delivery system polynucleotide and/or vector(s)) described elsewhere herein. In some aspects, the engineered cells can express one or more of the engineered delivery system polynucleotides and/or can produce one or more engineered eCIS systems, which are described in greater detail herein. Such cells are also referred to herein as “host cells,” “producer cells,” or “donor cells,” depending on the context. It will be appreciated that these engineered cells are different from “modified cells” described elsewhere herein in that the modified cells are not necessarily producer or donor cells (e.g., they do not make engineered eCISs) unless they include one or more of the engineered delivery system molecules or vectors described herein that render the cells capable of producing an engineered delivery vesicle. Modified cells can be recipient cells of an engineered eCIS and can, in some embodiments, be said to be modified by the engineered eCISs and/or a payload present in the engineered eCIS that is delivered to the recipient cell. The term “modification” can be used in connection with modification of a cell that is not dependent on being a recipient cell. For example, isolated cells can be modified prior to receiving an engineered delivery system or engineered eCIS and/or payload.

In an aspect, the invention provides a non-human eukaryotic organism; for example, a multicellular eukaryotic organism, including a eukaryotic host cell containing one or more components of an engineered delivery system described herein according to any of the described embodiments. In other aspects, the invention provides a eukaryotic organism; preferably a multicellular eukaryotic organism, comprising a eukaryotic host cell containing one or more components of an engineered delivery system described herein according to any of the described embodiments. In some embodiments, the organism is a host of lentivirus or AAV.

The engineered cell can be any eukaryotic cell, including but not limited to, human, non-human animal, plant, algae, and the like.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces. Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some aspects the engineered cell can be a cell line. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, ClR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Further, the engineered cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered delivery vesicle is produced it can package one or more molecules that are within the producer cell that can be related to the desired physiological and/or biological characteristic. In this context, the payload molecules incorporated into the delivery vesicles can be capable of transferring the desired characteristic to a recipient cell.

In some embodiments, a cell can be obtained from a subject, modified such that it is an engineered eCIS producer cell, and administered back to the subject from which it was obtained (autologous) or delivered to an allogenic subject. In other words, a producer cell described herein can be used in an autologous or allogenic context, such as in a cell therapy. In these embodiments, the cells can deliver a payload, such as a therapeutic payload or a payload that can manipulate a cellular microenvironment within the subject.

Conventional viral and non-viral based gene transfer methods can be used to introduce nucleic acids (e.g. such as one or more of the polynucleotides of the engineered delivery system described herein) in cells or target tissues. Such methods can be used to administer nucleic acids encoding components of a nucleic acid-targeting system to cells in culture, or in a host organism. In some aspects, a delivery is via a polynucleotide molecule (e.g. a DNA or RNA molecule) not contained in a vector. In some aspects, delivery is via a vector. In some aspects, delivery, is via viral particles. In some aspects, delivery is via a particle, (e.g. a nanoparticle) carrying one or more engineered delivery system polynucleotides, vectors, or viral particles. Particles, including nanoparticles, are discussed in greater detail elsewhere herein.

Vector delivery can be appropriate in some aspects, where in vivo expression is envisaged. It will be appreciated that the engineered cells can be generated in vitro, ex vivo, in situ, or in vivo by delivery of one or more components of the engineered delivery systems as described elsewhere herein.

Suitable conventional viral and non-viral based methods of engineering cells to contain and/or express the engineered delivery system polynucleotides and/or vectors described herein are generally known in the art and/or described elsewhere herein.

### Formulations

Component(s) of the engineered delivery system (e.g., eCIS), engineered cells, engineered delivery vesicles, or combinations thereof can be included in a formulation that can be delivered to a subject or cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, and combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In aspects where the pharmaceutical formulation contains one or more cells, the amount can range from about 1 cell to l x 10², 1×10³, 1×10⁴, 1×10⁵, 1×10⁶, 1×10⁷, 1×10⁸, 1×10⁹, 1×10¹⁰ or more cells. In aspects where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

### Pharmaceutically Acceptable Carriers and Auxiliary Ingredients and Agents

In aspects, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered delivery vesicles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g. the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, manitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration.

Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. Such unit doses may therefore be administered once or more than once a day. Such pharmaceutical formulations may be prepared by any of the methods well known in the art.

### EXAMPLES

### Example 1: Reconstituting and Characterizing an Endogenous eCIS Capable of Injecting Protein Payloads into Eukaryotic Cells

Photorhabdus Virulence Cassettes (PVCs) are eCISs produced by members of the genus Photorhabdus, which exist as endosymbionts within entomopathogenic nematodes. PVCs are secreted by bacterial symbionts and released by the nematode host, where they then target insects and inject toxin payloads; the resulting insect carcasses then support the growth of both the nematode and the Photorhabdus cells living within. PVCs exist as compact ˜20 kb operons and contain 16 core genes (pvc1-16) necessary for the assembly of the full injection system. In contrast to many other eCIS orthologs (including the metamorphosis-associated contractile structures, MACs, which were shown to kill murine cells), PVC payload genes exist immediately downstream of the core structural genes and do not contain any genes located in trans on the bacterial genome. Furthermore, Photorhabdus species also commonly harbor arrays of repeating PVC loci on the genome, with each individual PVC flanked by its own unique payload region, suggesting these microbes have evolved to target multiple different insects. This could perhaps be a strategy to expand the potential sources of nutrition for the bacteria-nematode symbiosis.

Consistent with the biological function of PVCs, endogenous payload genes span a diverse set of toxins. A number of putative toxin domains are associated within genes associated with PVC loci. Furthermore, putative PVC payloads exhibit significant variability in size, with some exceeding 1,000 residues in length.

As a first step in developing a targeted delivery tool based on eCISs, the inventors selected an eCIS system that can be reconstituted it in a genetically-malleable organism like Escherichia coli. As discussed above, the Photorhabdus Virulence Cassettes (PVCs), a type of eCIS found in the pathogenic microbe Photorhabdus, possess properties that make them convenient to study in the laboratory. To begin, PVCs have a simple and compact genetic architecture, allowing reconstructing these systems in the laboratory without the risk of accidentally missing critical genes located in trans on the bacterial genome (FIG. 1A). Furthermore, PVC payloads can also be easily identified by their predicted functions, as PVCs are known to inject proteins containing putative toxin domains.

The inventors cloned and expressed PVC particles in the laboratory; specifically, the entire PVC structural operons and payload regions from the genomic DNA of Photorhabdus cells were amplified using long-range PCR and cloned into low copy number expression backbones (to reduce unwanted mutations as a result of the large size of plasmids containing PVC loci). The inventors then set up a two-plasmid experiment in which E. coli cells were co-electroporated with one plasmid containing the PVC structural operon and another containing the associated payload region to initiate the production and assembly of PVC particles (FIG. 1). The 20 kb operon which encodes the PVC needle structure, along with the genomic region encoding payload proteins (FIG. 1A). Due to their high molecular weight, PVCs can be easily purified from E. coli lysate by pelleting them several times in an ultracentrifuge. The inventors then verified that all PVC components have been expressed via Coomassie stain (FIG. 1C), and purified PVC particles were verified for proper assembly with negative-stain TEM imaging (FIG. 1D).

After successfully purifying wildtype PVCs in the laboratory, the inventors began characterization of these systems by exposing them to cultured eukaryotic cells. Given the biological role of PVCs as toxin delivery systems that have evolved to target insects, the inventors wanted to test whether they will exhibit activity in their wildtype form against eukaryotic cells, so the inventors tested these systems in cultured insect cells (Spodoptera frugiperda Sf9 cells, a model insect cell line) (FIG. 1E). Because PVCs load toxin proteins, the injection efficiency of these systems was measured by measuring cytotoxicity, using a viability stain like fluorescein diacetate/propidium iodide (FDA/PI) or a luminescence-based assay like CellTiter Glo.

To supplement these assays, controls were constructed by knocking out PVC core genes thought to be important for successful PVC injection. Payload-deficient (i.e., unloaded) PVCs were generated by removing an ATPase gene (pvc15) that shares homology to ATPases necessary for genome packaging in contractile tail bacteriophages (FIG. 1H—ΔATPase (pvc15)). In addition, to control against the possibility that payload may be able to enter cells via a non-PVC-dependent mechanism (e.g. endocytosis), a PVC mutant that still successfully loads payload but is unable to inject target cells was constructed. For example, the creation of “blunted” PVCs via the deletion of the tail spike protein (pvc10) may interfere with the PVC tube's penetration of the host cell membrane. Additionally, deletion of the tail fiber protein (pvc13) resulted in PVCs that are unable to bind to target cells, as the tail fiber protein is known to mediate target cell recognition in other CISs but likely is not involved in payload loading (FIG. 1F—ΔFiber(pvc13)). PVCs without toxic payloads (FIG. 1F—Δpvc17/Δpvc21) also failed to kill the target cells. The inventors found that tail fiber mutant PVCs can still load payloads (toxin or Cre protein) (FIG. 1G). Finally, the inventors purified endogenous PVC payload proteins individually (i.e. through a separate procedure of affinity purification) and administer to target cells in the absence of PVC particles and showed that PVCs are in fact necessary for the delivery of these proteins into the cell (FIG. 1H).

The experiments described here highlight the utility of reconstructing eCISs in a genetically-tractable organism like E. coli, as opposed to in the endogenous producer organisms (e.g. Photorhabdus). All previous functional studies of eCIS activity have relied upon the production of eCISs in natural producers, but with this approach it is difficult to construct rigorous controls that improve confidence that the eCIS activity being described is genuine. In addition, organisms that produce eCISs often also produce a battery of other delivery systems, making it difficult to be certain that any activity seen in vitro is truly the result of the eCIS and not some other system. Finally, many previous studies of eCIS activity were done in live target organisms (for example, measuring metamorphosis in tube worms26 or toxicity in live moths), which made it difficult to rigorously quantify eCIS activity. The experiments described above- and in particular in the generation of a variety of mutants to produce predictable changes in eCIS activity, as well as in the use of insect cell lines as the eCIS target organism instead of live animals-represent the most thorough interrogation of eCIS activity to date.

### Example 2: Engineering Novel eCISs Capable of Injecting Custom Payloads into Custom Target Cells

While some eCISs have been found to show activity in eukaryotic cells, no successful attempts have been made to engineer these systems to achieve novel behavior. Any targeted delivery tool requires two basic elements of programmability: (1) the ability to modify the payload of the delivery tool, such that it can deliver novel payloads specific to the application in question, and (2) the ability to modify the target specificity of the delivery tool, such that it can deliver a payload into a defined set of cells and tissues. After the initial characterization of the activity of endogenous PVCs, the inventors worked on solving these two basic problems with regard to PVCs.

Because the engineered eCISs of the instant disclosure are to deliver molecular medicines and other useful payloads, the inventors first tested to swap out the toxin payloads that are naturally injected by these systems with novel proteins. To achieve this, the inventors characterized the mechanism by which payload proteins are loaded into PVCs. As discussed previously, it is not currently known how payload proteins are recruited by the PVC tube structure, but the inventors hypothesized that there exist signatures within endogenous PVC effectors that target them for loading into the PVC, either through direct binding to structural proteins or via chaperones that assist in the loading of the payload. Furthermore, the methods established in Example 1 allowed the inventors to make pinpoint modifications to PVC payloads while assessing the effects of these modifications on loading. In particular, because the instant purification scheme involves ultracentrifugation and therefore selectively purifies high molecular weight protein complexes, any unloaded free protein will not be included in the final purified protein product. As a result, the inventors can check whether a payload variant successfully loaded into the PVC by running western blots with samples that localized to the pellet during ultracentrifugation. See FIG. 2A. An Alphafold analysis revealed that the N-terminus of the payload is highly disordered. See FIG. 2B. Using this sample preparation method, the inventors identified a “packaging domain” within the PVC payloads by creating a series of truncation mutants of varying sizes and narrowing down regions that are both necessary and sufficient to load a protein into a PVC particle. See FIG. 2C. Beginning at either terminus of the payload protein, the inventors generated modified payloads with gradually more sequence removed.

After a certain amount of sequence was lost from either of the ends, the payload band on the western blot (indicating successful loading into the PVC) disappeared, indicating that a necessary portion of the packaging domain of the payload was deleted (FIG. 2C). The inventors found that a ˜60 residue motif on the N-terminus of the payload is both necessary and sufficient to load the protein into the PVC needle (FIG. 2C). These experiments provided valuable information about the mechanism of payload loading in PVCs; namely, it provided support for the proposition that payload loading in PVCs is specific and not merely promiscuous.

Furthermore, the identification of packaging domains sufficient for the loading of payloads into PVCs allowed the engineering of PVCs that load novel, arbitrary payloads.

After identifying regions within endogenous payloads that are sufficient to target them to the PVC, the inventors then attempted to package novel proteins into PVCs. In principle, PVCs may be able to accommodate a diverse array of potential payloads, because endogenous PVC payloads are remarkably diverse despite the core genetic architecture of the PVC syringe structure being mostly conserved. Using the same sample processing method as with the experiments with endogenous PVC payloads—i.e., co-expression of PVCs and payloads in E. coil, followed by purification and removal of unloaded proteins via ultracentrifugation the inventors investigated the ability of PVC packaging domains to load novel proteins not naturally loaded into PVCs. Alongside these designs, the inventors also generated two negative controls: (1) PVCs co-expressed with novel payloads fused to scrambled versions of the packaging domains (to show that payload packaging requires a functional packaging domain), and (2) PVCs with mutant payload loaders (pvc15) co-expressed with novel payloads containing functional packaging domains (to make sure that the packaging domain really mediates loading and not merely nonspecific binding to the PVC particle).

The inventors were able to successfully load GFP, Cre and Zinc Finger nuclease to the PVC syringe structure (FIG. 2D) by fusing a PVC packaging domain onto the N-terminus. The inventors also showed that PVCs can also load TALENs (FIG. 2E). These experiments constituted the first documented example of an eCIS harboring a non-endogenous payload protein.

After demonstrating that eCISs can load non-endogenous proteins, the inventors set out to show that these proteins can also be injected and produce functional consequences in target cells. The inventors loaded PVCs with Cre, a common payload for studies of delivery tools, by fusing it to a PVC packaging domain identified in the previous experiments. The inventors then assessed the ability of the PVC to deliver functional Cre protein by transfecting the target cells with a plasmid harboring a Cre reporter system alongside the PVCs loaded with Cre. The inventors used a double-floxed EGFP reporter to facilitate facile quantitation of PVC injection activity via flow cytometry. A double-floxed GFP plasmid would fluoresce in cells if the PVCs successfully deliver functional Cre protein. As with the previous experiment involving PVCs loaded with endogenous toxins, the inventors included a number of important controls to improve our confidence in the validity of the experiment involving novel payloads, including the use of “blunted” PVCs (deficient for the tail spike, pvc10), nontargeting PVCs (deficient for tail fiber, pvc13), and unloaded PVCs (deficient for payload loader, pvc15). The inventors found that Cre-carrying PVCs were able to deliver the novel non-endogenous Cre payload into sf9 insect cells (FIG. 2F). However, PVCs were unable to deliver the payload to Sf9 cells when tail fiber, payload loader (ATPase) or packaging domain was deleted (FIG. 2F). This experiment represents the first documented example of the delivery of functional novel payloads into target cells via an eCIS.

### Example 3: Targeting Human Cells with Engineered eCIS

In order to identify tropism-determining elements in PVCs and to generate modified particles capable of targeting human cells, the inventors focused on the tail fiber, a protein known to mediate host cell recognition in other CISs. Homology searches using endogenous PVC tail fibers reveal hypervariable domains in the C-terminus of this protein that are similar to those in phage tail fibers, suggesting this region may also be involved in target cell recognition. Tail fibers in phages are known to bind target cells via a C-terminal binding knob structure, and PVC tail fibers also contain this domain. Therefore, the inventors decided to rationally engineer PVC host recognition in a structure-guided fashion. The inventors fused antibodies and other binding domains to this binding knob region of the tail fiber and screened the resulting PVC particles as described above, except this time in human cells instead of insect cells (FIG. 3A-3C). The inventors further compared the targeting ability of different PVC particles in high EGFR-expressing A549 cells (FIG. 3D). WT PVC and PVC with a truncated tail failed to target and kill A549 cells. On the other hand, PVCs having the knob domain of human adenovirus 5 (hAd5 knob) and artificial receptor against human EGFR (E01 DARPin) successfully targeted and killed A549 cells (FIG. 3D). In addition, PVCs having a mutant knob domain (hAd5 knob (DL491-492) and a lysozyme-targeting domain (A4 DARPin) failed to target or kill A549 cells. See FIG. 3D. A similar experiment was conducted on HEK293T cells, which express low levels of EGFR. See FIG. 3E. The results showed that EGFR-targeting artificial receptor was less effective at killing HEK293T cells, presumably due to low EGFR expression. See, FIG. 3E. The inventors were also able to engineer PVCs to target cells displaying artificial receptors, such as cells displaying anti-HA, anti-FLAG, anti-EE, anti-MoonTag, anti-SunTag, and anti-ALFA tag scFvs (FIGS. 3F-3G). These experiments represented the first demonstration of how PVCs can be engineered to alter their behavior and target other cell types.

### Example 4: Applying eCIS In Vivo

As shown in Example 3, engineered eCIS is a versatile protein delivery tool that can deliver arbitrary proteins into defined target cells, including human cells. In order to study the in vivo activity of engineered PVCs, intraperitoneal injections of PVCs that have been loaded with Cre and retargeted against mammalian cells are performed.

To titrate the initial PVC dose for in vivo PVC activity (1) peripheral blood is periodically sampled and luciferase activity due to PVCs is measured, and (2) in vivo imaging is used to trace the localization of PVC injection of Cre within the animal. To make sure any observed in vivo signal is genuine, several PVC conditions that should be unable to efficiently inject Cre are tested, for example a nontargeting PVC (Apvc13, missing tail fiber) or an unloaded PVC (Apvc15, missing ATPase). Finally, a viral vector (e.g. AAV-Cre) is used to compare the activity of PVC injection to an industry standard.

After showing that engineered PVCs can be used to deliver custom payloads in live animals, the inventors are interested in expanding the set of possible applications for PVCs in real-world contexts. For example, one use for PVCs is in the delivery of genome editing technologies to facilitate the treatment of genetic diseases. Although PVCs may be particularly compatible with tools that are entirely composed of proteins (e.g. zinc finger nucleases, TALENs, etc.), it may be possible to artificially load ribonucleoproteins into PVCs to facilitate CRISPR genome editing in target cells. While PVCs do not naturally load nucleic acids, they do share significantly structural homology to other CISs that are known to deliver nucleic acids (e.g., phages). To achieve this, a gRNA is covalently fused to or non-covalently complexed with Cas9 harboring a PVC packaging domain, and subsequent to live animal administration guide-dependent cleavage by Cas9 is measured using deep sequencing.

Engineered eCISs (such as engineered PVCs) are also used as a novel platform for the development of targeted cancer therapies. A number of antibody-based targeted therapies have been developed in recent years, but these technologies are generally incapable of producing target cell death except as an indirect result of the recruitment of the host immune system. With engineered eCISs, it is possible to fuse cancer-specific antibodies to the targeting factor (tail fiber, pvc13) to allow the injection of toxins or peptide chemotherapies to kill the cell. By enclosing toxic drugs in a nanoscale syringe structure that injects its payload into cancer cells via a receptor-specific interaction, it is possible to reduce off-target interactions characteristic of traditional chemotherapies (i.e. toxicity in healthy host cells) and thus reduce side effects.

In addition, eCISs are used to develop targeted therapies for other diseases such as autoimmune disorders. eCISs are engineered to specifically target and kill immune cells showing activity towards self-antigens (e.g., self-reactive immune cells) by injecting toxins to those cells. Furthermore, eCISs are used to develop targeted therapies for viral diseases, such as by targeting eCISs toward viral glycoproteins and either disrupting virions by physical disruption (via PVC injection) or by injecting toxins into cells infected with the virus to thereby kill the infected cells.

Finally, the endogenous function of eCISs to produce toxicity in eukaryotic organisms—is used to develop applications in the area of targeted biocontrol. While antibiotics have proven remarkably powerful in combatting microbial infections for almost a century, there remains a scarcity of effective drugs against eukaryotic parasites and pathogens, and these drugs often produce severe side effects in patients. The originally-evolved function of eCISs are utilized to combat a variety of competing eukaryotic organisms for which satisfying treatments have remained elusive; for example, fungal pathogens, malaria, and multicellular parasites (e.g. worms, etc.). eCISs are also engineered to be used in agriculture as “smart” herbicides and insecticides that selectively kill pests or invasive species while sparing crops or healthy flora.

### Example 5: eCISs can Deliver Cargo Specifically In Vitro or In Vivo

eCIS-mediated delivery of base editors into human cells. Zinc finger deaminase (ZFD) was delivered to H1EK293T cells using eCIS. The cells targeted with the DL491-492 mutant eCIS (which cannot bind to target cells) did not display base editing (FIG. 5A). Next, spCas9 was delivered to HEK293T cells using eCIS. Successful indel production was observed when Cas9 was delivered together with a guide RNA (guide). The cells treated with the DL491-492 mutant eCIS (which cannot bind to target cells) did not display any indels (FIG. 5B).

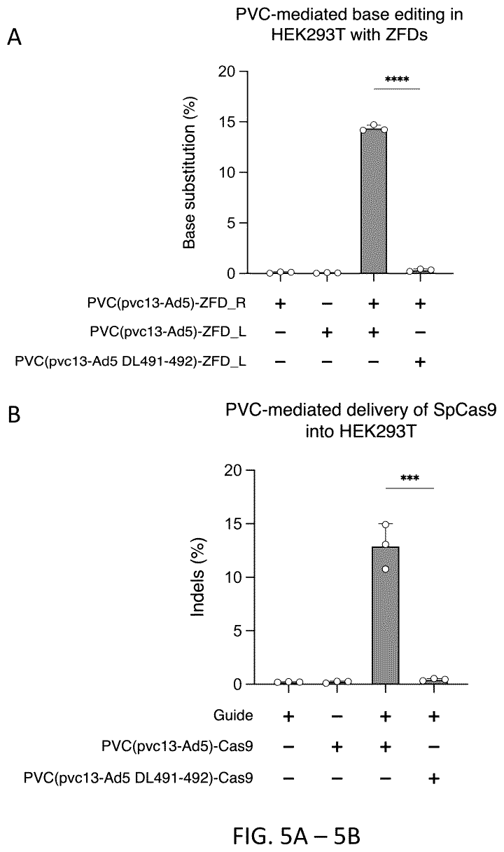

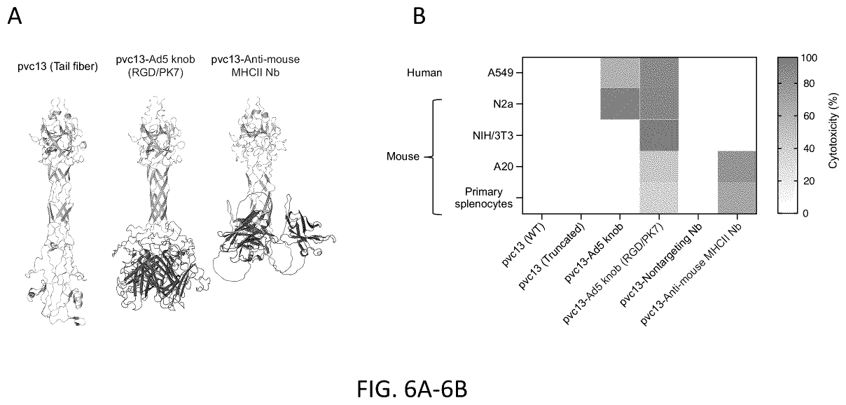

New eCIS designs enable delivery of cargo to mouse cells. Different eCIS designs were used as shown in FIG. 6A: pvc13 with WT tail fiber (pvc13(WT)), pvc13 with Ad5 binding domain having RGD and polylysine (PK7) domains (pvc13-Ad5 knob (RGD/PK7), which shows enhanced targeting properties, and pvc13 with anti-mouse MIC II nanobody (pvc13-anti-mouse MIC II Nb). See, FIG. 6A. Cytotoxicity assays were performed using the different eCIS constructs. Briefly, pvc13 with WT and truncated tail fiber domains were unable to target and kill human or mouse cells. Ad5 knob-targeted pvc13 were able to target human A549 cells; but they could only target and kill N2a mouse cells. On the other hand, super infective Ad5(RGD/PK7)-targeted pvc13 was able to target all cells tested. Mouse-cell specific targeting was observed using pvc13-anti-mouse MIC II Nb which targeted and killed MHCII expressing A20 cells and primary splenocytes. Mouse-cell specific targeting was not onserved with pvc13 having a nontargeting nanobody (pvc13-Nontargeting Nb). See FIG. 6B.

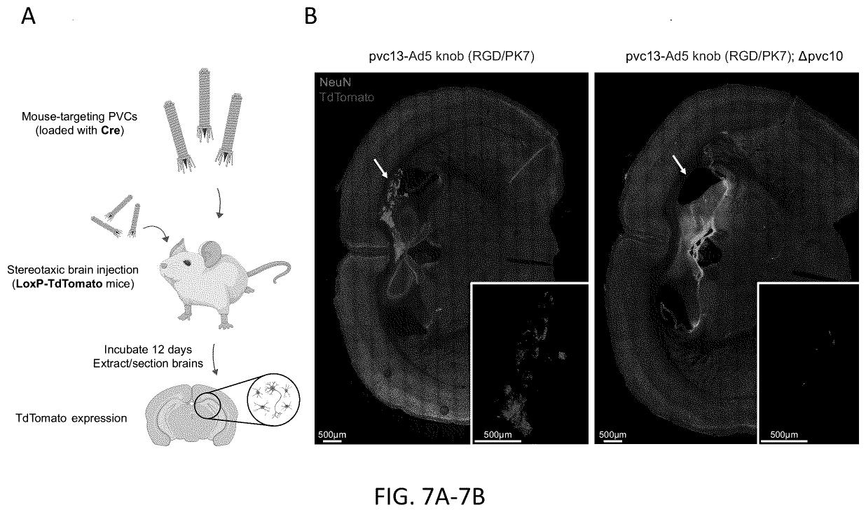

The new eCIS designs are active in vivo. FIG. 7A shows a schematic representation of the experiment. Briefly, mouse-targeting PVCs loaded with Cre were administered to LoxP-T dTomato mice via stereotaxic brain injection. 12 days later mice were sacrificed and their brain sections were imaged for TdTomato expression. See, FIG. 7A. Later, TdTomato expression was observed in mouse brains treated with pvc13-Ad5 knob (RGD/PK7), but not in mouse brains treated with a pvc13 version lacking the pvc10 structural gene encoding the spike protein (pvc13-Ad5 knob (RGD/PK7) Apvc10). See, FIG. 7B.

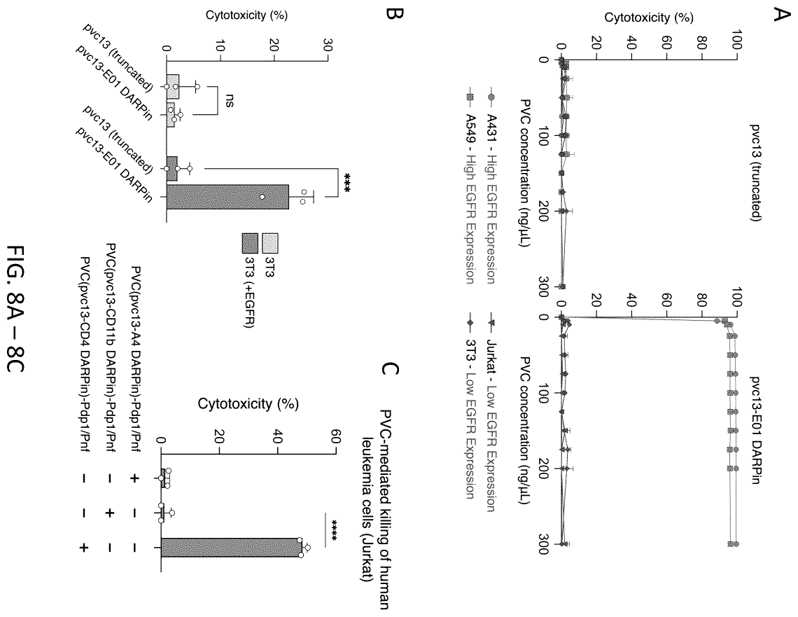

eCISs are highly specific. eCISs that have truncated tails (pvc13(truncated)) are unable to target and kill the cell lines tested. On the other hand, eCISs having an EGFR-specific targeting domain (pvc13-E01 DARPin) are able to target and kill cell lines that have high EGFR expression (e.g., A431 and A549) more than cell lines that have low EGFR expression (e.g., Jurkat and 3T3). See, FIG. 8A. eCISs having an EGFR-specific targeting domain (pvc13-E01 DARPin) are able to target and kill 3T3 cell line which are transfected with an EGFR construct. See FIG. 8B. Jurkat cells, which express high levels of EGFR, are only targeted and killed by eCISs expressing a targeting domain specific to a protein they express (CD4 in this instance). See FIG. 8C.

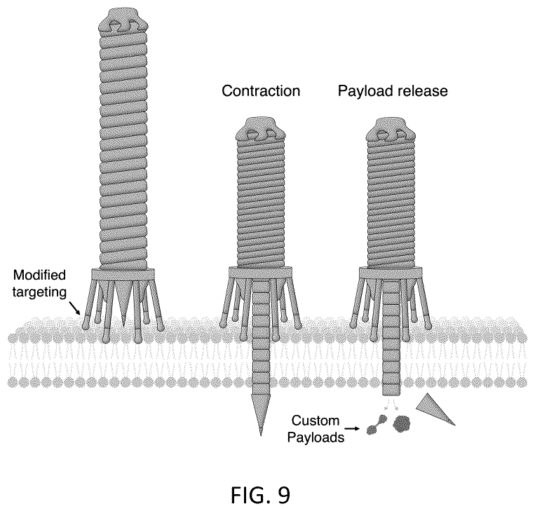

FIG. 9 shows a graphical representation of eCIS action. An eCIS binds to the target cell through the modified targeting domain. The eCIS then contracts and injects its custom payload into the target cell. See, FIG. 9.

### Example 6: Sequences

SEQ ID NO: 1-Pvc1 (tube) amino acid sequence:
MSTSTSQIAVEYPIPVYRFIVSVGDEKIPFNSVSGLDISYDTIEYRDGVGNWFKMPGQSQS
TNITLRKGVFPGKTELFDWINSIQLNQVEKKDITISLTNDAGTELLMTWNVSNAFPTSLTS
PSFDATSNDIAVQEITLMADRVIMQAV
SEQ ID NO: 2-Pvc1 (tube) nucleotide sequence:
atgtctacaagtacatctcaaattgcggttgaatatcctattcctgtctatcgctttattgtttctgtcggagatgagaaaattccatttaata
gtgtttcaggattagatattagttatgacaccattgaataccgagatggtgttggtaattggttcaaaatgccgggtcagagtcagagcactaa
tatcaccttgcgtaaaggcgttttcccggggaaaacagaactgtttgattggattaactctattcagcttaatcaggtagagaaaaaggatatt
accatcagtttaactaatgatgcaggtaccgaattattaatgacctggaatgtttctaatgcttttcccacttcattgacttcaccttcatttg
atgccaccagtaatgatattgcagtacaggaaattacgctgatggcagatcgggtgattatgcaggctgtttga
SEQ ID NO: 3-Pvc5 (tube connector-initiator tube) amino acid sequence:
MNDYYTPVVSHRFMASFIFNRIPDPLDIRFQRISGLSRELQVTQYSEGGENARNNYLAEK
IQHGTLTLERGVMTVSPLTWMFDRVLSGEKIAYADVVVMLLNENSLPLSSWTLSNALPV
RWQTSDFDANSNAILVNTLELRYQDMRWLGVKI
SEQ ID NO: 4-Pvc5 (tube connector-initiator tube) nucleotide sequence:
atgaacgattattacacacccgtggtatcccatcgttttatggcgagttttatttttaaccgcattcccgatccgctggatattcgttttcagc
gtatctctggccttagtcgggaactacaggtgactcagtacagtgagggaggagaaaatgcccgtaataactatttagctgagaaaatccaaca
cggtacgttgactttggaacggggcgtgatgacagtctcgccattgacctggatgtttgatcgggtattgagtggtgaaaaaatcgcttatgcc
gatgtggtggtgatgctactgaatgaaaattcactgccattgtccagttggacgttgagcaatgcgctgccggtacgctggcaaaccagcgact
ttgacgctaacagcaatgccatattggtgaatacccttgaattgcgttaccaggatatgcgctggcttggagtcaaaatatga
SEQ ID NO: 5-Pvc7 (tube connector-initiator tube) amino acid sequence:
MSLIERGLAKLTINAYKDREGKIRAGTLQAMYNPDSLQLDYQTDYQQSQAINSEKQSSI
YVQAKPAGLSLELIFDATMPGNKTPIEEQLMQLKQLCSVDATSNETRFLQVKWGKMRW
ESRGYFAGRAKSLSVNYTLFDRDATPLRVRVILALVADESLVLQETEQNLQSPAKIALRI
QDGVSLALMAASTASTLSGGVDYLTLAWQNGLDNLNGFVPGEILQATRGDES
SEQ ID NO: 6-Pvc7 (tube connector-initiator tube) nucleotide sequence:
atgagtctgattgaacgtggtttagctaagctgacaattaatgcttataaggatagggaagggaagatacgggcaggaacgttgcaggccat
gtataaccctgactccttgcaactggattaccaaacggattatcagcaatcccaagcgattaatagcgaaaagcaaagtagcatttatgtaca
ggccaagcccgcagggttatcacttgaattaatttttgatgccacgatgccgggtaacaaaacccccattgaagagcagctcatgcagctca
agcaactgtgcagtgtggatgcaaccagtaacgagacgcgattcctgcaagttaaatggggcaaaatgcgttgggaaagtcggggttacttt
gctggcagggccaagagtttgtctgtgaattacactttgtttgatcgtgatgcgactcccttgagggtacgggtaatattggcattagtggctg
atgaaagtctggtgttgcaggagactgaacaaaatctgcaatctccggcaaaaatcgcattacgcatacaggatggggtatctctggctctgat
ggcagccagtacggcatcaacattgtcaggcggtgtggattatctgacgctggcctggcaaaacggtctggataatctcaatgggttcgttc
cgggtgaaatattgcaggccaccaggggagacgaatcatga
SEQ ID NO: 7-Pvc2 (sheath protein-major sheath unit) amino acid sequence:
MTTVTSYPGVYIEELNSLALSVSNSATAVPVFAVDEQNQYISEDNAIRINSWMDYLNLIG
NFNNEDKLDVSVRAYFANGGGYCYLVKTTSLEKIIPTLDDVTLLVAAGEDIKTTVDVLC
QPGKGLFAVFDGPETELTINGAEEAKQAYTATPFAAVYYPWLKADWANIDIPPSAVMA
GVYASVDLSRGVWKAPANVALKGGLEPKFLVTDELQGEYNTGRAINMIRNFSNTGTTV
WGARTLEDKDNWRYVPVRRLFNSVERDIKRAMSFAMFEPNNQPTWERVRAAISNYLY
SLWQQGGLAGSKEEDAYFVQIGKGITMTQEQIDAGQMIVKVGLAAVRPAEFIILQFTQD
VEQR
SEQ ID NO: 8-Pvc2 (sheath protein-major sheath unit) nucleotide sequence:
atgacaaccgttaccagttatcctggcgtttatattgaagaattaaatagcctggccttgtcagtttcaaatagcgccacagcggttcctgttt
ttgctgtggacgaacaaaaccaatatattagtgaagataatgcaatccgtattaattcgtggatggattatcttaatctgattggcaattttaa
taatgaagacaaattagatgtttctgtgcgtgcttattttgccaatggaggtggatattgttatctcgtcaaaacaacgagtttagaaaaaatt
attccaaccttggatgatgtaaccttattggttgctgcgggcgaagatattaaaacgacagtagatgttttatgtcagccaggaaaagggttat
tcgcagtctttgatggccctgaaacagagttgactatcaacggtgcggaagaggcaaaacaagcctataccgccacaccattcgctgcggttta
ttatccttggttgaaagcggattgggctaacatagatattccacccagtgcagtgatggcgggagtttatgcatcggtggatttatcccgtggt
gtatggaaagcgcctgccaatgttgcgttgaaagggggcctggaacctaaatttttagtcacggatgaattgcagggtgaatataacactggcc
gcgctatcaatatgattcgtaatttcagtaacacaggtactacggtttggggtgcaagaaccctggaagataaagacaattggcgttatgttcc
agtgcgacgcttgtttaattctgtggagcgggatatcaagcgtgccatgagctttgctatgttcgagcctaataatcagcctacttgggagcgg
gtacgggcggcgattagcaactacctttatagcctgtggcaacaggggggattagctggcagcaaagaagaagacgcttattttgtgcaaattg
gtaaaggtataacgatgacacaggagcagattgatgcagggcaaatgattgttaaagtcggtttggctgctgtacggcctgcggaatttatcat
tctccagtttacgcaagatgtagaacagcgttaa
SEQ ID NO: 9-Pvc3 (sheath connector protein-minor sheath unit) amino acid
sequence:
MSAILKAPGVYIEEDASLALSVSNSATAVPVFIGKFTPTVVDSIQVCTRISNWLEFTSSFSL
APTVEIVVQSNTESESESETYHYIETINLSPAVEALRLYFQNGGGACYIYPLNDAEDEL VL
AAIPEVIEQKGDITLLVCPELDLDYKTKIYGAVSSLLNDNKVGYFLIADSNDGESVSGVW
NSAKAAAYYPQLETNLKFSTLPGDKDIRISGYQDDDETHKPKNLDELRTINEALAQDIDA
RLLEEKQRAVIIPPSAAIAGIYCQTDNRRGVWKAPANVALTGIGSLLDKVDDERQGEMN
DKGINVIRSFTDRGFMVWGARTCVDAANISWRYIPVRRLFNSVERDIRQALRAVLFETN
SQPTWVRAKAAVDQYLYTLWQKNALMGARPEEAYFVQIGQDITMSEADIKQGKMIMT
VGLAAVRPAEFIILQFTQDVVQ
SEQ ID NO: 10-Pvc3 (sheath connector protein-minor sheath unit) nucleotide
sequence:
atgtctgctattctgaaagcgcctggcgtttatattgaagaagacgcttccctagcgttgtctgtcagtaacagcgcgactgccgtgcctgttt
ttatcggaaaatttactccgacagtggttgattcaatccaagtctgtacccgtatcagcaactggcttgaattcacttcctctttttccctagc
tccaacagttgagattgttgtccaatctaacactgaatctgaatctgaatctgaaacttaccactatattgagacaatcaatttatctccagct
gtggaagcattgcgactctattttcaaaatggcggaggagcttgctatatctacccattaaatgatgctgaagatgaattggttctggcggcca
taccagaagtcattgaacagaaaggtgatattactctgttggtttgcccggaactcgatctggattacaaaactaagatctatggcgcagtgag
ctcactgttgaatgataacaaagtgggctatttcctgattgcggatagcaatgatggagaatctgtgtcaggagtatggaatagtgctaaggcc
gccgcctattatccccagttggaaactaacctaaaattttccacgttgcctggggataaggacattcgtatcagcggttatcaggatgatgatg
aaacacataaaccgaaaaacttggatgagctcaggacaatcaacgaggcgttggcacaggatattgatgcaagattgctcgaggagaaacaacg
tgctgtcatcattccgccaagtgctgccattgcgggcatttattgccaaacggataatcgtcgcggtgtttggaaagcgccagccaacgttgcg
ctcacagggatcgggagtttgcttgataaggtagacgatgaacggcagggagagatgaatgacaagggaatcaatgtcatccgttcatttaccg
accgtggttttatggtctggggagcccgtacttgtgtggacgctgccaacatcagctggcgttatattcctgttcgtcgcctgttcaattccgt
tgaacgagatatccgccaggcgctgcgcgctgtgttgtttgaaactaatagtcagcctacctgggtacgtgctaaggctgccgttgatcaatat
ctttataccctttggcagaaaaatgcattgatgggtgctcgcccggaagaagcttattttgtgcaaattggtcaggatatcaccatgtccgagg
ctgatattaaacagggtaagatgatcatgactgttggtttggcagcagtgcggccagctgagttcatcattctgcaatttacgcaggatgttgt
tcagtaa
SEQ ID NO: 11-Pvc4 (sheath connector protein-minor sheath unit) amino acid
sequence:
LTVPTLTILEEVMMMERLQPGVTLTESIITMGQQEIPSAVPVFIGYTVRYPEQSEASVRID
SLAEYTSLFGDDHVMMFAVRHYFDNGGQQAFVLPLKDNMPSVEMTTAEAENLIAALRS
ATVSEAIGGHSQITLILVPDMARLNDSDIDDSSTQVSLWSQGWEALLQLSQVRPNLFVLL
DAPDNVEQAQKCMTTLSSDYRQWGAAYWPRLETTYQKEISGKDNESQGIFQGTVLSPT
AAVAAVIQRTDNDAGVWKAPANIALSQVIRPVKSYLQGSVLFNSSGTSLNVIRSFPGKGI
RVWGCRTLENTDNTQWRYLQTRRLVSYVTAHLTQLARMYVFEPNNELTWMKLKGQS
YNWLRQLWLQGGLYGSQEDEAFNILLGVNETMTEDDVRAGKMIMKVELAVLFPAEFIE
ISLVENTQTEALS
SEQ ID NO: 12-Pvc4 (sheath connector protein-minor sheath unit) nucleotide
sequence:
ttgacagtgcctactctaaccatcttggaggaggtgatgatgatggagagactccaaccgggtgtgactttaacagaaagtataatcacgatg
ggtcagcaagagatacccagtgctgtgccggtgtttattggttacaccgttcgttatccggaacaatcggaagcatcagtccgtatcgacagtt
tggccgagtataccagcctgtttggtgacgaccatgtgatgatgtttgctgtcaggcactattttgataatggcgggcaacaggcatttgtttt
acccctgaaggacaatatgccatcagtggagatgaccacagctgaagcggaaaatctgatagccgcattgcgctctgctacggttagcgaagc
cattggtgggcatagtcagattacactgattttggtaccggatatggctcggcttaatgacagtgatattgatgactcctcaacccaggtaagc
ctgtggtcccaaggctgggaggcgctgctgcaattgagtcaggttaggcccaacctctttgtgctgttagatgcgccggataatgttgaacag
gcgcagaagtgtatgacaacgctatcgtcagattatcgtcaatggggggcagcatattggcctcgtctggaaactacctatcagaaagaaat
atctggcaaggacaatgaatctcagggaattttccaggggactgttctgtcacccacagccgcggtcgcagcggtaattcaacgcacggat
aacgacgcgggtgtttggaaagcaccggccaatattgccttatcccaggttattcgacctgttaaatcttatcttcagggaagtgtactgttta
acagcagcggcacttcgctcaatgtgatccgcagtttcccaggtaagggcatacgggtatggggatgccgcactctggaaaacacggataat
acgcagtggcgctatctgcaaacacgtcggctggtttcctatgtaacagcgcatttgacccaattggctcgcatgtatgtctttgagccaaata
atgaacttacctggatgaagttaaaaggacaaagttacaactggttacggcaattatggttgcagggtggcttgtatggttcacaggaggatg
aggcatttaacattctgttaggcgtaaacgagacgatgactgaggatgatgttcgtgcaggaaaaatgatcatgaaagttgagttggctgtgtt
gtttcctgccgaatttattgagatcagtttggtgtttaatacccaaacagaggcgctgtcttaa
SEQ ID NO: 13-Pvc11 (baseplate subunit) (AA) amino acid sequence:
MELNELTNKLSNLVPMTDFKLDNRASLQLLKYIEAYTKIIPFNSGDKYWNDFFFMSGNT
PEKLAKLYQKEIEPNGELLPQQAFLLAVLRLLETPISLLNVLPAAHRELYYRELLGLSSHA
AQPDQVALSMELNSTVMEQLLPEGTLFEAGQDEQGNALQYALDASLLANRGYISDLRW
LRNDGEKQWVTSAPWDLQAQVSLPSDGIRLFGKTNSDQQVFGGVLITSSLLAMEAGIRK
IIVTFEQEMNTQELVAQVSSGNQWLTLTSEVNKKEVTLTLSDKEPAISAPEDLDNLFFTQ
PVLRLQGKDSQALPEVTGISVSEKDDTKDTSFEMYHLTPFGYSSDIEPLEENPALYLGFT
DVKPGQTLALYWKLKSPQQPTVSWYYLDQHNQWAELDSWVSDGTQNLYQDGTWHV
ELPVDASNQAEQMPVGRYWLRAVVEVPAHEGALGKAPWLYGLIYNAMTATLVNVDSI
SDSHELTPLPASSIQRPVEPIIVLASVNQPWASWGGRIPESYSAFFERIAQNLSHRNRSLTW
GNMVTLLKERYVSIFDVKYPGNDELTRVPALEQQQLTVIPANRYNDSDDSLRPVLNPAR
LQEMADWLQQKDSPWASIEVRNPEYLDVKIHYEVIFKPDVNEDFGYRQLQQQLCEVYM
PWSIDEQRPVVLNNSINYFQLLATIQQQPLVERVTRLTLHRADSSDESDGTASVEAKDNE
VLILVWEEDDNLQYRGNDYE
SEQ ID NO: 14-Pvc11 (baseplate subunit) (DNA) nucleotide sequence:
atggaattaaatgagttaactaacaaattgtcaaatttggtgccaatgaccgattttaaattagataatcgagccagtttgcaattgcttaaat
atattgaagcgtatacgaagataataccctttaattctggcgataaatattggaatgactttttctttatgtcaggaaatacgccagagaaact
tgcaaaattatatcagaaagaaatagaacccaatggggagttattacctcagcaggcttttttgttggcggttttgcgtttattggaaacacca
atatccttattaaatgtattacctgctgctcatcgtgagctctattatcgggagcttttaggcttgtcttcccatgcggcacagcctgatcagg
ttgctttatctatggaactgaattcgacagtgatggaacagctgctccctgaaggaaccctgtttgaggctggtcaggatgaacaaggcaatgc
attgcaatatgccctggatgccagtttgctggctaatcgtggatatatcagtgacttgcgctggttacggaatgacggggaaaagcaatgggtt
acttctgctccatgggatttacaggcacaggtgtcactgccgtctgatgggatacgattatttggtaagacaaatagtgatcagcaggtatttg
gtggggtgttgataacgtcatcacttctggcgatggaagcggggataaggaagatcattgttacttttgagcaggagatgaacacccaagaact
ggtggcacaggtcagcagtggaaatcaatggctaacattgacgtctgaggtaaataagaaagaggtcacactgacactgtcagacaaagaaccg
gcaatcagtgcgccagaggatctggataatctctttttcacgcaaccggtactcaggctacagggaaaggatagtcaggcactgccggaggtga
cgggtatcagcgtttcggaaaaggatgatactaaggatacctcttttgagatgtatcacttaacaccatttggttatagcagtgatatagagcc
attggaggaaaatccagcgttatatttaggctttactgatgtaaagccagggcaaacactggcgctgtattggaaattaaaatccccgcagcaa
ccaaccgtttcctggtattacctggatcaacataatcaatgggctgaattggattcatgggtcagtgatggaacccagaatctgtatcaggatg
gtacttggcacgttgagttgcctgtggatgcatccaatcaggcagagcagatgccagttggacgctattggttgcgggcagtggtggaggta
cccgctcatgagggggcgttggggaaggctccttggctatatggtctaatctataacgccatgacggcaaccttggttaatgtagatagcatc
agtgacagccatttcttaacccctttgcctgccagcagcatacagcggcccgttgaacccatcattgtgttggcatcggtcaaccagccttgg
gcatcatggggggacgtatacctgaatcctacagtgccttttttgaacggatagctcaaaacctgtctcatcgaaaccggtccttaacctggg
gaaatatggtgacattactcaaagagcgttatgtcagcatctttgatgttaagtatccaggtaatgatgaactcaccagagtgccagcattgga
gcagcagcaactaacagtgattccagcaaaccggtacaacgatagcgatgattctctgcgtccggtactgaatcctgctcgtctgcaagaga
tggctgattggttgcagcagaaagactctccctgggcctctattgaggtcaggaatccagaatacttggatgtgaaaatccattacgaggtga
tttttaaacctgatgtgaacgaagattttggctatcgccagctacagcagcaactgtgtgaggtgtatatgccttggagcatagatgagcagcg
gcccgttgtattgaataacagcattaattatttccagttgttagccactattcaacagcaaccgctggttgagcgagtcactcgtctgacacta
catcgggctgattcttctgatgagagtgatggtacagcatctgtggaagccaaagataatgaagtgcttattttagtctgggaagaggacgata
atctgcaataccgaggaaatgactatgagtaa
SEQ ID NO: 15-Pvc12 (baseplate subunit) (AA) amino acid sequence:
MSNQDALFHSVKDDIHFDTLLEQAHQVIEKQAEKLWSDTAEHDPGITFLQGISYGVSDL
AYRHTLPLKDLLTPAPDEQQQEGIFPAEFGPHNTLTCGPVTADDYRKALLDLHSSDSLD
GTQQDEGDFLFRSVQLVREPEKQRYTYWYDATKREYSFVNSEGAKEFTLRGNYWLYLE
PTRWTQGNIAAATRQLTEFLTKNRNIGESVSNIIWLQPVDLPLLLDVELDDDVGAQDVP
GIFAAVYSTAEQYLMPGAQRYRTEVLQNAGMSNDQIFEGPLLEHGWIPELPAARDYTQR
LTLNLSRLVNSLLEIEGIKHVNRLRLDDSFDKTAIEPVKGDTWSWSIKEGYYPRLWGEDP
LNQLAQQNGPLRVIAKGGISVSVSKEQIQASLPSQSLIQNEPVILAYGQHRDVGSYYPVS
DTLPPCYGLQHSLSESEHLLPLHQFMLPFEQLLACGCQQIAMLPRLLAFQREGYEVWGD
QWPFKSGSVNDDAHQDYAPALKDLLGQIALDSDHELDIINYLLGYFGTQRAPRTFTTQL
DDFRAVQQGYLAQQPTLTYHRSNIRIDQVSSLQKRIAARMGLGGELFKPQPDLSQLPFY
LIEHRALLPVKPNSQFDKEQKPASVTEEGGSQTGQHYVVIEQKGIDGKLTQGQVINLILY
EGEQGETQFTIRGQMVFKTEGDKFWLDVNNSAQLEYNLARVMTAAKASKLFWQNSPV
WMEDMGYRLAYASDQSSLPVNQRRLTRTVQTPFPPMVVVGSEITLLKQVGIVNLKKAE
SEKLYAKVVSFDRIEGTLIIERLGNSTLAFPTSEEAWRYSWYFSGEKYERTDRFSFVISVV
VNSDLIKLPGVDPYKLEEWVKETILTEFPAHISMIIHWMDREAFLNFANTYQRWQNNGT
PLGDAAYSILESLTLGKLPSALKGVGTMRIATSSQREEVVGSNGDQWNTDGITQNELFY
VPKES
SEQ ID NO: 16-Pvc12 (baseplate subunit) (DNA) nucleotide sequence:
atgagtaatcaggatgcactgtttcatagcgttaaagacgatattcactttgataccttgctggaacaagctcatcaggtgattgaaaaacagg
ctgaaaaactgtggagtgatacggcagagcatgatccgggtatcacatttttgcagggaatcagttacggtgtgtcagatttggcttaccgac
atacattacccctgaaagatttactgactccggcgccggatgagcagcagcaagagggaatttttcctgccgaatttggcccgcataatacac
tgacttgtgggccggtgacagcggatgattatcgcaaggcattgttagatctacacagcagcgacagcctggatggtactcagcaggatga
gggggattttctgttccggagtgtgcaactggtgcgtgaaccggaaaaacagcgttatacctattggtatgatgcaaccaagagggaatata
gctttgtcaacagtgaaggggctaaagagtttaccttgcgggggaattactggttgtatctggaaccaacccgttggactcagggtaatattgc
cgctgctaccagacaactgacagaatttttgactaaaaatcgcaatattggtgaatctgtcagcaacattatctggctacaaccggttgatctg
ccactgttgctggatgttgaactggatgatgatgtaggtgcacaggatgtccccggtatttttgcggcggtgtatagcaccgcagagcagtatc
tgatgcctggagcacagcgttaccgtacggaagtactgcaaaatgctgggatgagcaatgatcaaatcttcgaaggtccattattggaacatg
gctggataccagagctgccggcagcccgtgattatactcaaaggctcactctcaatcttagccggttggtaaatagtctgcttgagattgagg
gcattaaacatgtgaatcgtcttcgtctggatgatagcttcgataaaactgctattgaacccgttaagggggatacctggtcgtggtcgatcaa
agagggctattatccacgtctttggggagaagacccacttaaccaattggcgcaacaaaatggcccgcttagggtgatagccaaaggagg
gattagcgtcagtgtgagtaaagagcaaatccaggccagtttacccagtcaatcactgattcaaaatgagccggtaatattggcttacggcca
gcaccgtgacgttggcagctattatcccgtcagtgatactttgccgccttgctatggactacaacattctttgtctgaaagtgaacacttattg
ccacttcatcaatttatgttgccatttgaacaattattggcctgtggttgtcaacagatagccatgctcccgcggttactggcttttcagcgcg
aaggttatgaggtttggggtgatcagtggccctttaagtcaggctcagtgaatgatgacgcccatcaagattatgcccctgcattaaaggattt
gttaggacagattgcgctggatagtgatcatgaattggatattattaattacttgctgggttactttggcacacagcgggcaccgcgtaccttt
acgacacaactcgatgattttcgtgcggtccaacagggttatctggcccagcaaccgacattgacttaccaccgctccaatattcgtatcgatc
aggtatcgtcgctacaaaaacgtattgctgctcgcatggggctgggcggtgagttgtttaaacctcaaccggatctgagccaactgccttttta
tttgattgaacatcgagcgttgctgccagtcaaacccaatagtcagtttgataaggaacagaaaccagcctcggtgacagaggaggggggcagc
caaacaggtcaacattatgtggtcattgaacagaagggcattgatggcaagctgacacaggggcaagtgatcaatttaattctgtatgaaggag
agcagggagaaacccaatttacgatacgcggtcagatggtattcaaaaccgagggggataagttttggttggatgtgaataatagtgcgcaa
ctggaatataatctggcgcgggtaatgacagcagccaaggcgagtaaactcttttggcaaaacagcccggtatggatggaggatatgggct
atcgtctggcctatgctagtgaccaatcctcattgcctgtgaatcaacggcgcttgacccgcacagtgcaaactccattcccgccgatggttgt
tgtaggtagcgaaatcaccctgttaaagcaggtggggatagtcaatTtaaaaaaagcggagtcagaaaaactttatgcaaaagttgttagctt
tgatcgcattgaagggaccttgattattgagcgtttgggtaattccactctggcttttcctacctcggaagaggcgtggcggtatagttggtat
ttttcgggggagaaatatgaaaggactgaccgcttttcatttgtgattagcgtagtagtgaacagtgacttaattaaattgcccggtgttgatc
cctataaattggaagaatgggtgaaagaaacgattcttaccgaatttccagctcatatttctatgattatccattggatggatcgggaagcctt
tttaaatttcgccaatacctatcagcgttggcaaaataatggtacgccactgggggatgcggcttattccattctagaaagtttgacacttggt
aaattgccatctgccttaaaaggtgttggcacaatgcgtattgccacatctagtcaaagagaagaagtggtgggtagtaatggtgatcaatgga
atacagatggaataacccagaatgaattattctatgttcctaaagagagctag
SEQ ID NO: 17-Pvc13 (tail fiber) (AA) amino acid sequence:
MNETRYNATVQEQQTLSNPKAVGPDIDKLKDKFKEGSIPLQTDFNELIDIADIGRKACGQ
APQQNGPGEGLKLADDGTLNLKIGTFSNKDFSPLILKDDVLSVDLGSGLTNETNGICVGQ
GDGITVNTSNVAVKQGNGISVTSSGGVAVKVSANKGLSVDSSGVAVKVNTDKGISVDG
NGVAVKVNTSKGISVDNTGVAVIANASKGISVDGSGVAVIANTSKGISVDGSGVAVIAN
TSKGISVDNTGVAVIANASKGISVDGSGVAVIANTSKGISVDGSGVAVIANTSKGISVDSS
GVAVKVKANGGIKVDANGVAIDPNNVLPKGVIVMFSGSTAPTGWALCDGNNGTPNLID
RFILGGKGTDINGVSTNTASGTKNSKLFDFSSDEATLTIDGKTLGRALSLQQIPNHAHFSG
IIMDTEKVNYYGSKKITTNVWGVTTGDNTSVRYIYKSSGVLDSNNNVSNSTLGGNSLQT
HDHDIKITGTGKHSHKNKVTVPYYILAFIIKL
SEQ ID NO: 18-Pvc13 (tail fiber) (DNA) nucleotide sequence:
atgaacgaaactcgttataatgcaactgtacaagaacaacaaacattatctaatccaaaagctgttggacctgacatcgataaattaaaggata
aatttaaagagggcagtattcccctgcaaaccgatttcaatgagttaattgatattgccgatattggacgtaaagcctgtggtcaagcgccaca
acaaaatggcccaggagaaggattgaaattggctgatgacggtacgcttaatttaaaaataggcactttttccaataaagacttttctccatta
atattaaaagatgatgttttatctgtagatcttggtagtggtctgactaatgaaaccaatggaatctgtgtcggtcagggcgatggtattacag
ttaacactagcaatgtagctgtaaaacaaggtaacggaattagcgttactagtagtggtggtgttgccgttaaagttagtgctaataagggact
tagcgttgatagtagtggtgttgcagttaaagttaatactgataagggaattagcgttgatggtaatggtgttgcagttaaagttaatactagt
aaaggaattagcgttgataatacaggtgttgcagttatagctaatgctagtaagggaattagcgttgatggtagtggtgttgcagttatagcta
atactagtaaaggaattagcgttgatggtagtggtgttgcagttatagctaatactagtaaaggaattagcgttgataatacaggtgttgcagt
tatagctaatgctagtaagggaattagcgttgatggtagtggtgttgcagttatagctaatactagtaaaggaattagcgttgatggtagtggt
gttgcagttatagctaatactagtaaaggaattagcgttgatagtagtggtgttgcagttaaagttaaagctaatggcggaattaaagtagatg
ctaatggtgttgcaattgatcctaataatgtactccccaagggagtgattgtaatgttctctggcagtactgcaccaactggttgggcgttatg
tgatggcaataatggtacaccaaatttaatcgatcgatttattttaggtgggaaagggactgatattaatggagtgagtactaatacagcttca
ggtactaaaaatagtaagttattcgatttcagttctgatgaagctacattaactattgatggtaaaacactggggagagcattatcgttacagc
aaatacctaatcatgcacactttagtggaataattatggatacagagaaagttaattattatggaagtaaaaaaatcacaacaaatgtgtgggg
tgtaacaacaggagataatacttcagtacgatatatttataagtcatcaggtgtacttgactctaacaataatgtctccaacagtaccttaggc
ggaaacagtctgcagacgcacgatcatgatattaagataacgggcacaggaaaacattctcacaaaaacaaagtaacagtcccttattatattc
tggctttcatcataaagctttaa
SEQ ID NO: 19-Pvc8 (spike) (AA) amino acid sequence:
MSHQLKIIADGKALSLLAAVDVDTCYRVNSIPSATLKLSVPDRPLSSFSQTDVQTELAHC
QVGKTLRLELIDGSKKWVLFNGLITRKALRIKNKQLLLTLVVKHRLQLMVDTQHSQLFK
DKSEKAILSTLLNQTGINARFGKIAALDQKHEQMVQFRCSDWHFLLCRLSATGAWLLPA
IEDVQFVQPDALKSNSAYTLKSRGDENKDIVVKDAYWQFDNQINPALLEVSGWDISKQ
QVQSGGRYGKIALGKAALSPDGLASLNKTGWDICYSSPLTTQESGYLAQGLLLNQRISG
VTGEFLLKGDGRYQLGDNIQLTGFGSQLDGTASITEVRHRLNRRIDWETTVSIGLQHEYL
PILPDAPELHIATVAKYQQDSAVLNRIPIILPVLNRPNEFLWARLGKPYASHESGFCFYPE
PGDEVIIGFFENDPRYPVILGAMHNPKNKAPFEPTQDNREKVLIVKKGEAQQQLVIDGKE
KMIRINAGENQIMLQQDKDISLSTKKELTLKAQTMNATMDKSLAMSGKNSVEIKGAKIN
LTQ
SEQ ID NO: 20-Pvc8 (spike) (DNA) nucleotide sequence:
atgagccaccaactgaaaattattgcagatggtaaggcactgtcacttttggccgcggtagatgtggacacctgttatcgggttaacagtatac
cttctgcgacattgaaactgagcgtaccggataggccactctcttctttcagtcagacggatgttcagacagaactggcccactgtcaggtag
ggaaaaccctgcgtctggaattgattgatggtagcaaaaaatgggtgctgtttaatggtcttattacccgtaaggctctgagaattaagaataa
gcaattattgctcactctggttgtcaagcatcggttgcaactgatggtggatacccagcattcacagctgtttaaagacaaaagcgaaaaagc
gatcttaagcacgctattgaatcagaccggaatcaatgctcgcttcggaaagatagcggcgttagatcaaaagcatgaacagatggtgcaat
ttcgttgttcagactggcattttctgttgtgccgactgtcggcaaccggtgcatggttgttacctgccatagaagacgttcagtttgttcaacc
tgatgctctgaaatcaaactcagcctataccttgaagagcaggggggatgagaacaaagacatcgttgtcaaggatgcttactggcagtttgac
aatcaaatcaaccccgctttgctggaagtcagtggctgggatatcagtaagcagcaggtacaatcaggcggtcgctacggaaaaatcgcgtt
gggtaaggcggcactctctcctgatggattggcatcccttaataaaacgggttgggacatttgttatagcagtccgttaacaacccaggaaag
cggttatctggcacagggattattgcttaaccagcgcatttctggggtgacaggagaatttttgctcaaaggagatgggcgttaccagttggg
agacaacattcagctgactggatttggttcacagttagatggtacggcaagcattactgaggttcgccaccgtcttaatcggcgaattgattgg
gaaaccacggtgagcattggtttacaacatgaatatttgccgatattacctgatgctcccgaactacatattgcgacagtagcgaaatatcagc
aggacagtgcggtgttaaaccgtatccccattattctgccggtactgaatcgtcccaatgaatttttgtgggccagattggggaaaccttatgc
tagccatgaaagcggtttctgtttttacccagagccaggtgacgaagttattattggtttttttgaaaatgatccgcgttatccagttatttta
ggtgctatgcataatccgaaaaataaggccccttttgaaccaacccaagataatagggaaaaagtattgatcgttaaaaaaggtgaagcgcaac
aacaattagtcattgatggcaaagagaaaatgatccgaattaatgcgggtgaaaatcaaataatgcttcagcaagataaagacatttctctgtc
aacgaaaaaagaattaacactgaaagcgcagacaatgaatgccacgatggataaatcattggcaatgtccgggaaaaacagtgttgaaatcaaa
ggcgcaaaaattaatcttacccaatga
SEQ ID NO: 21-Pvc10 (spike tip) (AA) amino acid sequence:
MSEAIVVDGDVLQFDPNFGNRQVTVPSPGKISGTGHAQVSGKKVCILGDEKQVRVSAT
YITTTHTTPGTGTITISALDAGQQALQCTSGAALIIKGQQFTAMFTPELPAMNNTVTPPQP
DVTTPSSGKGRFITQQNFATVN
SEQ ID NO: 22-Pvc10 (spike tip) (DNA) nucleotide sequence:
atgagtgaagcgattgtggtggatggtgacgtgttacagtttgatcccaactttggcaatcggcaggtgacggttcccagcccaggaaaaatt
agcggcacaggacatgcgcaggtaagtggaaaaaaagtgtgtattctgggggatgagaaacaggtcagggtttctgcaacctatattacaa
caacacatactacgccgggaacaggaaccattactatcagtgctctggatgctggccagcaggcccttcagtgtaccagtggggggcttt
aattatcaaggggcagcaatttacggcgatgtttacgcctgaattgccagccatgaataatacagtgactccgccacaaccggatgttacgac
accttcatcaggaaaaggacgttttatcactcaacaaaattttgctaccgtaaattag
SEQ ID NO: 23-Pvc16 (terminal cap-sheath/tube terminator) amino acid sequence:
MLNTQTIIDVNKAMDAMLRAYLNQDIAIRFDLPELDTMQSDAMVSIFLYDIHEDLQLRS
AESRGFDVYAGRLLPGWVNIKCNYLITYWEASKPATDASSPDSQPDNQAIQVMSQVLN
ALINNRQLAGIPGAYTQVVPPKESLNSLGNFWQSLGNRPRLSLNYSVTVPVSLNDGQDS
ATPVTAVSSTVEQTASLSQEVVSHALRELLITELGGGEDNRLVLSKVELSAVKETMTQD
SPAQMIILLSVSGITRQEYLKEIDNIFDRWVNNAEVITTIDDCGIRIESITKDNLVGI
SEQ ID NO: 24-Pvc16 (terminal cap-sheath/tube terminator) nucleotide sequence:
atgttaaacacgcaaactattattgatgtcaataaggcaatggatgccatgctgcgcgcatatctgaatcaagatattgccattcgttttgatc
tacctgaattggatactatgcaatctgatgcgatggtaagtatctttctttatgacattcatgaagatttacagcttcgctcggcagaatcaag
agggtttgatgtttatgccgggaggttattgcctggttgggtaaatattaaatgtaactatctgattacctattgggaagcttctaagccagcg
actgatgccagcagtccggatagccaacctgataaccaggcaatacaagtgatgtcacaagtattaaatgccttgattaataatcgtcaattgg
caggtattcctggtgcttatactcaggttgtaccgcctaaagagagtttaaatagcctggggaatttctggcaatcactgggtaatcgcccacg
gctttctctcaattattcagtgacagtacctgttagcctaaacgatggtcaggatagcgcgactccggttaccgcggtttcttctacagtggaa
caaacggcatcgctcagtcaagaagtggttagtcatgctttacgcgaattactcattacggaattaggaggaggagaggataaccggttggtac
tgagtaaagttgaattatccgcagtgaaagagacgatgactcaagacagtccggctcagatgattatattgttgtctgtttcaggcattacacg
acaggaatatttgaaggaaattgataatatctttgatcgttgggtaaataatgctgaagttattaccactattgatgattgtgggattagaatt
gaaagtataacgaaagataatcttgtaggaatttaa
SEQ ID NO: 25-SepC_NTD packaging domain amino acid sequence:
MPRYANYQINPKQNIKNSHGKSSSSDFSSGYLSFSNNSLDDPFIRQQVKREFIWEGHMKE
IEEASRL
SEQ ID NO: 26-SepC_NTD packaging domain nucleotide sequence:
atgcctagatatgctaattatcagataaaccccaaacagaatattaaaaattcacatgggaaatcttcatcgtcagatttttctagtgggtacc
tttcattctcaaataattcgcttgatgacccttttattcggcagcaagttaagagagaatttatttgggagggacatatgaaggagattgaaga
agcttcaagatta
SEQ ID NO: 27-Pnf_NTD packaging domain amino acid sequence:
MLKYANPQTVATQRTKNTAKKPPSSTSFDGHLELSNGENQPYEGHKIRKIKGLRQHLAD
R
SEQ ID NO: 28-Pnf_NTD packaging domain (DNA) nucleotide sequence:
atgttaaaatatgctaatcctcagaccgtagccacacaacgtactaaaaatactgcgaagaaaccgccatcatcaacctcttttgatgggcac
cttgaactttcaaatggtgaaaaccagccttacgaaggccataagattaggaaaatcaaaggattgagacaacatcttgcggatagg
SEQ ID NO: 29-3xGGSGG linker amino acid sequence:
GGSGGGGSGGGGSGG
SEQ ID NO: 30-3x GGSGG linker (SEQ ID NO: 29) (DNA) nucleotide sequence:
ggtggCtctggtggtggCggttctggCggCggtggttctggCggt
SEQ ID NO: 31-pvc15 amino acid sequence:
MNISPVFYDSLNQDNDRDLSFLFSELERIDLALQHHFYCVESQRSELLDEFLLTEAEVVT
RLDKPLGKPHWINDDYLAISQKGNVSLMAASRLMDLIERFELTDFERDVLLLGLLPHFD
SRYYRLFSLIQGGQQGRLPSFALALELFCHSALEKQVQQASFLHRAPLMGCQLLSIDTSQ
KTLAWLQTPFITDSGVYHFLLGHHYIMPALEHCAEWLTPTGIGCYPEGLKQVLGNVLLS
DNDNIRPIVLLRGMAGSARAYTITNMMASEGKQTLLVDISKLADSDEKNIILQIKHILRET
RMHGACLLLRNFCLLVEQNKQLLDSLSELLNQPELRIVCLIEPYSPLVWLKKIPVLLIEMP
LLTPAEKARLLIASLPDNCSEDIDTITLSQRYTFNPETLPLILQEAQLYQQQRDPLDILQQC
DIRQALNLRAQQNFGQLAQRIIPKRSLKDLLVSDEIAQQLREILIAIKYREQVLAGGFKDK
IAYGTGISALFYGDSGTGKTMAAEVIADHIGVDLIKVDLSTVVNKYIGETEKNLSRIFDLA
EQDAGVLFFDEADALFGKRSETKDSQDRHANIEVSYLLQRLENYPGLVILSTNNRGHLD
SAFNRRFTFITRFTYPDEKIRKKMWQEIWPRNIKISEDIDFNELAQRTSVTGANIRNIALLS
SFFASEQGNDEVSNENIEIALKRELAKVGRLTF
SEQ ID NO: 32-pvc15 nucleotide sequence:
atgaatatatcgcctgttttttatgattcattgaatcaggataacgaccgtgatctatcgtttttatttagcgaactggaacgaatagatctcg
ctcttcaacaccatttttattgtgtagaaagtcagcgaagtgagctcctggatgagtttctgctcactgaggcggaagtggtgaccaggctgga
taagccacttggtaaacctcattggataaatgatgattatctggcgatatcgcaaaagggcaatgtaagcctaatggcagcgtccagattaatg
gatctgatcgaacgctttgaactgactgattttgagcgcgatgttttactattaggcttattgccccattttgatagccgctattatcgactgt
tttcgctgattcaagggggacaacagggtcgattaccttcttttgcgctggcattggaactgttttgccactcggcgctggagaaacaggtaca
gcaagcgagttttctgcaccgggcacctttgatgggttgccagctattatccatcgatactagtcaaaaaacgctggcctggctccagactccc
tttattactgacagcggggtatatcactttttactggggcatcactacattatgccggctttagaacattgtgctgagtggttaacaccgacag
ggattggctgttatcctgaaggattaaaacaagtactgggtaacgtattgttatctgacaacgataatattagaccgattgtcttattacgggg
aatggccggcagtgccagagcttataccattactaatatgatggcttcagaagggaagcaaacactgctggtagatatatccaaacttgctgat
agcgatgaaaaaaacattattcttcagataaagcatattttgcgggaaacccgcatgcatggagcatgtttattattacggaatttttgcttgt
tagtggaacagaataaacaactattggactccctgtcagagttattgaatcaacctgaattaagaattgtttgcctgattgagccttattcccc
attggtatggctgaaaaagataccggtattactgattgagatgccacttttaacgcctgcggaaaaagccagattgttaattgccagcttaccg
gataattgttccgaggatattgatacgataactttaagccagcgttacacttttaacccagaaaccctgccattgattttgcaagaggcccagc
tttatcaacagcagcgagatccgctggatatcttgcagcaatgcgatatacgccaggcattaaatttgcgtgctcaacaaaatttcggtcaatt
ggcacagcggattattcctaagcgctcattaaaggatttattggtatccgatgagattgctcagcagttacgggaaatactcatagcaattaag
tatcgggaacaggttctggcgggagggtttaaagataaaattgcctatggcactggtatcagcgccctgttttatggtgattcaggcactggaa
aaaccatggcagcagaagtgattgctgaccacattggcgttgacttaataaaagtggatttatctacagtagtgaataaatacatcggtgaaac
agaaaaaaacttatcccgtattttcgatttggcggaacaggatgcaggggtattattctttgatgaagctgacgcactgtttggtaaacgcagt
gaaactaaagattcccaggacagacatgccaatattgaagtttcttacttattacagcgcctggagaattacccgggtctggtcattttatcca
ccaataatcgtggtcatttagacagtgcttttaatcgtcgttttactttcattacccgttttacttacccggatgaaaaaatccgtaaaaaaat
gtggcaggaaatttggcctagaaatataaaaatatcggaagatatcgattttaacgaattagctcaacgaacaagcgtgactggcgcgaatatc
cgcaatattgctttattgtcttcattctttgcttcagagcaggggaatgatgaagtcagtaatgaaaatattgaaattgcattgaagcgtgaat
tagctaaagtcggacgattaacattttaa
SEQ ID NO: 33, Protein, Artificial sequence
PKKKRKV
SEQ ID NO: 34, Protein, Artificial Sequence
PKKKRKVEAS
SEQ ID NO: 35, Protein, Artificial Sequence
KRPAATKKAGQAKKKK
SEQ ID NO: 36, Protein, Artificial Sequence
PAAKRVKLD
SEQ ID NO: 37, Protein, Artificial Sequence
RQRRNELKRSP
SEQ ID NO: 38, Protein, Artificial Sequence
NQSSNFGPMKGGNFGGRSSGPYGGGGQYFAKPRNQGGY
SEQ ID NO: 39, Protein, Artificial Sequence
RMRIZFKNKGKDTAELRRRRVEVSVELRKAKKDEQILKRRNV
SEQ ID NO: 40, Protein Artificial Sequence
VSRKRPRP
SEQ ID NO: 41, Protein, Artificial Sequence
PQPKKKPL
SEQ ID NO: 42, Protein, Artificial Sequence
SALIKKKKKMAP
SEQ ID NO: 43, Protein, Artificial Sequence
DRLRR
SEQ ID NO: 44, Protein, Artificial Sequence
PKQKKRK
SEQ ID NO: 45, Protein, Artificial Sequence
RKLKKKIKKL
SEQ ID NO: 46, Protein, Artificial Sequence
REKKKFLKRR
SEQ ID NO: 47, Protein, Artificial Sequence
KRKGDEVDGVDEVAKKKSKK
SEQ ID NO: 48, Protein, Artificial Sequence
RKCLQAGMNLEARKTKK
SEQ ID NO: 49, Protein, Artificial sequence
MSTDATLIRTTPSHAEADATDTLVATPLMPPRRVISPWPGPGEGQSLMRIPVVDIRGMAL
MPCTPAKARHLLKSGNARPKRNKLGLFYVQLSYEQEPDNQSLVAGVDPGSKFEGLSVV
GTKDTVLNLMVEAPDHVKGAVQTRRTMRRARRQRKWRRPKRFHNRLNRMQRIPPSTR
SRWEAKARIVAHLRTILPFTDVVVEDVQAVTRKGKGGTWNGSFSPVQVGKEHLYRLLR
AMGLTLHLREGWQTKELREQHGLKKTKSKSKQSFESHAVDSWVLAASISGAEHPTCTR
LWYMVPAILHRRQLHRLQASKGGVRKPYGGTRSLGVKRGTLVEHKKYGRCTVGGVDR
KRNTISLHEYRTNTRLTQAAKVETCRVLTWLSWRSWLLRGKRTSSKGKGSHSS
SEQ ID NO: 50, Protein, Artificial sequence
MQPAKQQNWVFQINGDKQPLDMINPGRCRELQNRGKLASFRRFPYVVIQQQTIENPQT
KEYILKIDPGSQWTGFAIQCGNDILFRAELNHRGEAIKFDLVKRAWFRRGRRSRNLRYR
KKRLNRAKPEGWLAPSIRHRVLTVETWIKRFMRYCPIAWIEIEQVRFDTQKLANPEIDGV
EYQQGELQGYEVREYLLQKWGRKCAYCGTENVPLEVEHIQSKSKGGSSRIGNLTLACH
VCNVKKGNLDVRDFLAKSPDILNQVLENSTKPLKDAAAVNSTRYAIVKMAKSICENVK
CSSGARTKMNRVRQGLEKTHSLDAACVGESGASIRVLTDRPLLITCKGHGSRQSIRVNA
SGFPAVKNAKTVFTHIAAGDVVRFTIGKDRKKAQAGTYTARVKTPTPKGFEVLIDGAR
ISLSTMSNVVFVHRSDGYGYEL
SEQ ID NO: 51, Protein, Artificial sequence
MAVFVIDKHKRPLMPCSEKRARLLLERGRAVVHRQVPFVIRLKDRTVQHS
AVQPLRVALDPGSRATGMALVREKNTVDTGTGEVYRERIALNLFELVHRG
HRIREQLDQRRNFRRRRRGANLRYRAPRFDNRRRPPGWLAPSLQHRVDTT
MAWVRRLCRWAPASAIGIETVRFDTQRLQNPEISGVEYQQGALAGCEVRE
YLLEKWGRKCAYCGAENVPLEIEHIVPKSRGGSDRVSNLALACRACNQAK
GNRDVRAFLADQPERLARILAQAKAPLKDAAAVNATRWALYRALVDTGLPVEAGTGG
RTKWNRTRLGLPKTHALDALCVGQVDQVRHWRVPVLGIRCAGRGSYRRTRLTRHGFP
RGYLTRNKSAFGFQTGDLIRAVVTKGKKAGTYLGRIAIRASGSFNIQTPMGVVQGIHHR
FCTLLQRADGYGYFVQPKPTEAALSSPRLKAGVSSAGN
SEQ ID NO: 52, Protein, Artificial sequence
MTTNVVFVIDTNQKPLQPCSAAVARKLLLRGKAAMERRYPAVIILKKEVDSVGKPKIEL
RIDPGSKYTGFALVDSKDNADFIIWGTELEHRGAAICKELTKRSAIRRSRRNRKTRYRKK
RFERRKPEGWLAPSLQHRVDTTLTWVKRICKFVPIMSISVEQVKFDLQKLENSDIQGIEY
QQGTLAGYTLREALLEHWGRKCAYCDVENVFLEIEHIYPKSKGGSDKFSNLTLACHKC
NINKGNKSIDEFLLSDHKRLEQIKLHQKKTLKDAAAVNATRKKLVTTLQEKTFLNVLVS
DGASTKMTRLSSSLAKRHWIDAGCVNTTLIVILKTLQPLQVKCNGHGNKQFVTMDAYG
FPRKSYEPKKVRKDWKAGDIIRVTKKDGTMLMGRVKKAAKKLVYIPFGGKEASFSS
ENAKAIHRSDGYRYSFAAIDSELLQKMAT
SEQ ID NO: 53, Protein, Artificial sequence
MPNKYAFVLDSKGKLLDPTKSKKAWYLIRKGKASLVEEYPLIIKLKREVPKDQVNSDKL
ILGIDDGTKKVGFALVQKCQTKNKVLFKAVMEQRQDVSKKMEERRGYRRYRRSHKRY
RPARFDNRSSSKRKGRIPPSILQKKQAILRVVNKLKKYIRIDKIVLEDVSIDIRKLTEGREL
YNWEYQESNRLDENLRKATLYRDDCTCQLCGTTETMLHAHHIMPRRDGGADSIYNLIT
LCKACHKDKVDNNEYQYKDQFLAIIDSKELSDLKSASHVMQGKTWLRDKLSKIAQLEIT
SGGNTANKRIDYEIEKSHSNDAICTTGLLPVDNIDDIKEYYIKPLRKKSKAKIKELKCFRQ
RDLVKYTKRNGETYTGYITSLRIKNNKYNSKVCNFSTLKGKIFRGYGFRNLTLLNRPKG
LMIV
SEQ ID NO: 54, Protein, Artificial sequence
MLFNKCIIISINLDFSNKEKCMTKPYSIGLDIGTNSVGWAVITDNYKVPSKKMKVLGNTS
KKYIKKNLLGVLLFDSGITAEGRRLKRTARRRYTRRRNRILYLQEIFSTEMATLDDAFFQ
RLDDSFLVPDDKRDSKYPIFGNLVEEKVYHDEFPTIYHLRKYLADSTKKADLRLVYLAL
AHMIKYRGHFLIEGEFNSKNNDIQKNFQDFLDTYNAIFESDLSLENSKQLEEIVKDKISKL
EKKDRILKLFPGEKNSGIFSEFLKLIVGNQADFRKCFNLDEKASLHFSKESYDEDLETLLG
YIGDDYSDVFLKAKKLYDAILLSGFLTVTDNETEAPLSSAMIKRYNEHKEDLALLKEYIR
NISLKTYNEVFKDDTKNGYAGYIDGKTNQEDFYVYLKNLLAEFEGADYFLEKIDREDFL
RKQRTFDNGSIPYQIHLQEMRAILDKQAKFYPFLAKNKERIEKILTFRIPYYVGPLARGNS
DFAWSIRKRNEKITPWNFEDVIDKESSAEAFINRMTSFDLYLPEEKVLPKHSLLYETFNV
YNELTKVRFIAESMRDYQFLDSKQKKDIVRLYFKDKRKVTDKDIIEYLHAIYGYDGIELK
GIEKQFNSSLSTYHDLLNIINDKEFLDDSSNEAIIEEIIHTLTIFEDREMIKQRLSKFENIFDK
SVLKKLSRRHYTGWGKLSAKLINGIRDEKSGNTILDYLIDDGISNRNFMQLIHDDALSFK
KKIQKAQIIGDEDKGNIKEVVKSLPGSPAIKKGILQSIKIVDELVKVMGGRKPESIVVEMA
RENQYTNQGKSNSQQRLKRLEKSLKELGSKILKENIPAKLSKIDNNALQNDRLYLYYLQ
NGKDMYTGDDLDIDRLSNYDIDHIIPQAFLKDNSIDNKVLVSSASNRGKSDDFPSLEVVK
KRKTFWYQLLKSKLISQRKFDNLTKAERGGLLPEDKAGFIQRQLVETRQITKHVARLLD
EKFNNKKDENNRAVRTVKIITLKSTLVSQFRKDFELYKVREINDFHHAHDAYLNAVIAS
ALLKKYPKLEPEFVYGDYPKYNSFRERKSATEKVYFYSNIMNIFKKSISLADGRVIERPLI
EVNEETGESVWNKESDLATVRRVLSYPQVNVVKKVEEQNHGLDRGKPKGLFNANLSS
KPKPNSNENLVGAKEYLDPKKYGGYAGISNSFAVLVKGTIEKGAKKKITNVLEFQGISIL
DRINYRKDKLNFLLEKGYKDIELIIELPKYSLFELSDGSRRMLASILSTNNKRGEIHKGNQI
FLSQKFVKLLYHAKRISNTINENHRKYVENHKKEFEELFYYILEFNENYVGAKKNGKLL
NSAFQSWQNHSIDELCSSFIGPTGSERKGLFELTSRGSAADFEFLGVKIPRYRDYTPSSLL
KDATLIHQSVTGLYETRIDLAKLGEG
SEQ ID NO: 55, Protein, Artificial sequence
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVH
NVNEVEEDTGNELSTKEQISRNSKALEEKYVAELQLERLKKDGEVRGSINRFKTSDYVK
EAKQLLKVQKAYHQLDQSFIDTYIDLLETRRTYYEGPGEGSPFGWKDIKEWYEMLMGH
CTYFPEELRSVKYAYNADLYNALNDLNNLVITRDENEKLEYYEKFQIIENVFKQKKKPT
LKQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG
SEQ ID NO: 56, Protein, Artificial sequence
KYSIGLDIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWDPK
KYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGD
SEQ ID NO: 57, Protein, Artificial sequence
MDPIRSRTPSPARELLSGPQPDGVQPTADRGVSPPAGGPLDGLPARRTMSRTRLPSPPAPS
PAFSADSFSDLLRQFDPSLFNTSLFDSLPPFGAHHTEAATGEWDEVQSGLRAADAPPPTM
RVAVTAARPPRAKPAPRRRAAQPSDASPAAQVDLRTLGYSQQQQEKIKPKVRSTVAQH
HEALVGHGFTHAHIVALSQHPAALGTVAVKYQDMIAALPEATHEAIVGVGKQWSGAR
ALEALLTVAGELRGPPLQLDTGQLLKIAKRGGVTAVEAVHAWRNALTGAPLN
SEQ ID NO: 58: Protein, Artificial sequence
RPALESIVAQLSRPDPALAALTNDHLVALACLGGRPALDAVKK
GLPHAPALIKRTNRRIPERTSHRVADHAQVVRVLGFFQCHSHP
AQAFDDAMTQFGMSRHGLLQLFRRVGVTELEARSGTLPPASQ
RWDRILQASGMKRAKPSPTSTQTPDQASLHAFADSLERDLDAP
SPMHEGDQTRAS
SEQ ID NO: 59: Protein, Artificial sequence
VITYF
SEQ ID NO: 60, Protein, Artificial sequence
VVVVV
SEQ ID NO: 61, Protein, Artificial sequence
KPWWPRR
SEQ ID NO: 62, Protein, Artificial sequence
NIVNVSLVK
SEQ ID NO: 63, Protein, Artificial sequence
Leu-Pro-Phe-Phe-Asp
SEQ ID NO: 64, Protein, Artificial sequence
KLVF
SEQ ID NO: 65, Protein, Artificial sequence
LVEALYL
SEQ ID NO: 66, Protein, Artificial sequence
RGFFYT
SEQ ID NO: 67, Protein, Artificial sequence
PPKKARED
SEQ ID NO: 68, pvc6 DNA, Species: Photorhabdus asymbiotica
Atgacagtagaaatcagagagttacttatccaggcaaaggtagtgccatcaacacgaccgactgaatcagaacggcaaaaccattctttga
tacaggaaagtctggatgaggcgacttgggtggaaacgataaaacgcgaagtgttggccgcattacgcgatgaggaagggtggcgtccat
ga
SEQ ID NO: 69, pvc6 protein, Species: Photorhabdus asymbiotica
MTVEIRELLIQAKVVPSTRPTESERQNHSLIQESLDEATWVETIKREVLAALRDEEGWRP
SEQ ID NO: 70, pvc9 DNA, Species: Photorhabdus asymbiotica
Atggaaaatcaaatactgacacaactctatggtcgtggttgggcttttcctccggtcttttcccttgaaaagggggtagagatggctgaaggg
gcggaagatgtgagacaaagtttgcagattctgtttagtactgagccgggggaacgtcttatgcgtgaaaattatggctgcggattaaatgatt
ttatgtttgaaaatatccgcaatgaacttattgctgaaattgaatcccatatccatgacaacgtattacgatatgaaccccgggctgatatgac
tgatattcaggttcgtcaatcccctggcatggggaatactttgcaagtgcaggtcatgtatcgcctgagagggagtgatatcaatcaacaaatc
cagggagtacttgcactgagtgaaggccgggtgacggaggtagtatga
SEQ ID NO: 71, pvc9 protein, Species: Photorhabdus asymbiotica
MENQILTQLYGRGWAFPPVFSLEKGVEMAEGAEDVRQSLQILFSTEPGERLMRENYGC
GLNDFMFENIRNELIAEIESHIHDNVLRYEPRADMTDIQVRQSPGMGNTLQVQVMYRLR
GSDINQQIQGVLALSEGRVTEVV
SEQ ID NO: 72, pvc14 DNA, Species: Photorhabdus asymbiotica
atgacttcggagccaaatctgttaaaccggattacaattactattgaagctaataatcaacaagtagctagaaaagtattgcatggctccttgc
ttaatcaagctaatataaataaattatttaattcatactttaatgaatatgaaattaataggggtgtttatttagaaacattaatcctgaatct
tggtacgataaatttccatgattttaattcattgtttcctactctcctaaaagctgcattgaataaagaattcagtcaatatcagataaacaac
catagggaagaaatgctatttaatgagacaatatcaaatcaagctactgataagtcttacatatttggcgataacaaattaattgatgcagaga
atttcattcactttttatatcaaaagcattccacattaaatctagtagaagcaatgggaaataatggtattgaaaaattaacaaatcagttaac
acaaatagaaaataaatttgcgttattattggcaaaaagttgtttgtctgaggaaggcttaaaacgactcttggctatcaaacaacccgattta
ttaatcgctatcaatcgcagattatctgaaagaataaatagaccacaatatcaggagaagcttgtttcctgcggacaactgatatttagtgctc
tgggatatatacaacagtacaatatacaggaaattcctaaaccggatgaaaaagttattgcacgcataacaactgaacttaataataatggttt
gcttaatacaatacctattattacactatttcgtcagagtgggattaacgattcatcactaaatgattggctaaagaaaatctggcaggtgaga
tcaatttcacagttatgcagaaagtatctttctgctaaggaataccaatatctgtcagaacattttgtttcaaagagcgtcgataaaaatagat
atgatgaagagcccgtaaatcagagcatattatcaaggttgaataataattccattaaagaaggaaataatcacagtcaactctgtactctcag
tagactatattctgaacccgttgtattacctgaacaaaccattctacgtcaggttagtaatacagtagatcagagcatattatcaaggttgaat
aatgcctccattaaagaaggaaataaccaaagtcaacttcgcactctcagtagactatattctgagcccgttgcattacctgaacaaaccattc
cacgtcaggttagtaatacaggtatattaattctatggccaatgctacctacactatttaaccagcttggtctacttgagaaaaagaaatttat
ccatcgtcaggcccagtttaatgccgttgattttcttgattacctgatttggggaaccgaagatgtgaaagtggaacgaaaggttttgaataat
gttctatgtgggttaatggctgatgaaattactgaaccaatgcctattgaaccagaaaaacaatggataataattcaatggctggacgctatta
tctcccaactttctggctggaaaaagttaagtcgtaatgacgtccgtcaattatttctacaacgaccaggagaattactgatcaatgaacagga
aattaaaatcacaatacagcaacaaccatttgatgctctgttaactgattggccgtggccaatgaatatggcttgttttagctggttgagtcaa
ccattaaccattacgtggttataa
SEQ ID NO: 73, pvc14 protein, Species: Photorhabdus asymbiotica
MTSEPNLLNRITITIEANNQQVARKVLHGSLLNQANINKLFNSYFNEYEINRGVYLETLIL
NLGTINFHDFNSLFPTLLKAALNKEFSQYQINNHREEMLFNETISNQATDKSYIFGDNKLI
DAENFIHFLYQKHSTLNLVEAMGNNGIEKLTNQLTQIENKFALLLAKSCLSEEGLKRLLA
IKQPDLLIAINRRLSERINRPQYQEKLVSCGQLIFSALGYIQQYNIQEIPKPDEKVIARITTE
LNNNGLLNTIPIITLFRQSGINDSSLNDWLKKIWQVRSISQLCRKYLSAKEYQYLSEHFVS
KSVDKNRYDEEPVNQSILSRLNNNSIKEGNNHSQLCTLSRLYSEPVVLPEQTILRQVSNT
VDQSILSRLNNASIKEGNNQSQLRTLSRLYSEPVALPEQTIPRQVSNTGILILWPMLPTLFN
QLGLLEKKKFIHRQAQFNAVDFLDYLIWGTEDVKVERKVLNNVLCGLMADEITEPMPIE
PEKQWIIIQWLDAIISQLSGWKKLSRNDVRQLFLQRPGELLINEQEIKITIQQQPFDALLTD
WPWPMNMACFSWLSQPLTITWL
SEQ ID NO: 74, pvc15 DNA, Species: Photorhabdus asymbiotica
atgaatatatcgcctgttttttatgattcattgaatcaggataacgaccgtgatctatcgtttttatttagcgaactggaacgaatagatctcg
ctcttcaacaccatttttattgtgtagaaagtcagcgaagtgagctcctggatgagtttctgctcactgaggcggaagtggtgaccaggctgga
taagccacttggtaaacctcattggataaatgatgattatctggcgatatcgcaaaagggcaatgtaagcctaatggcagcgtccagattaatg
gatctgatcgaacgctttgaactgactgattttgagcgcgatgttttactattaggcttattgccccattttgatagccgctattatcgactgt
tttcgctgattcaagggggacaacagggtcgattaccttcttttgcgctggcattggaactgttttgccactcggcgctggagaaacaggtaca
gcaagcgagttttctgcaccgggcacctttgatgggttgccagctattatccatcgatactagtcaaaaaacgctggcctggctccagactccc
tttattactgacagcggggtatatcactttttactggggcatcactacattatgccggctttagaacattgtgctgagtggttaacaccgacag
ggattggctgttatcctgaaggattaaaacaagtactgggtaacgtattgttatctgacaacgataatattagaccgattgtcttattacgggg
aatggccggcagtgccagagcttataccattactaatatgatggcttcagaagggaagcaaacactgctggtagatatatccaaacttgctgat
agcgatgaaaaaaacattattcttcagataaagcatattttgcgggaaacccgcatgcatggagcatgtttattattacggaatttttgcttgt
tagtggaacagaataaacaactattggactccctgtcagagttattgaatcaacctgaattaagaattgtttgcctgattgagccttattcccc
attggtatggctgaaaaagataccggtattactgattgagatgccacttttaacgcctgcggaaaaagccagattgttaattgccagcttaccg
gataattgttccgaggatattgatacgataactttaagccagcgttacacttttaacccagaaaccctgccattgattttgcaagaggcccagc
tttatcaacagcagcgagatccgctggatatcttgcagcaatgcgatatacgccaggcattaaatttgcgtgctcaacaaaatttcggtcaatt
ggcacagcggattattcctaagcgctcattaaaggatttattggtatccgatgagattgctcagcagttacgggaaatactcatagcaattaag
tatcgggaacaggttctggcgggagggtttaaagataaaattgcctatggcactggtatcagcgccctgttttatggtgattcaggcactggaa
aaaccatggcagcagaagtgattgctgaccacattggcgttgacttaataaaagtggatttatctacagtagtgaataaatacatcggtgaaac
agaaaaaaacttatcccgtattttcgatttggcggaacaggatgcaggggtattattctttgatgaagctgacgcactgtttggtaaacgcagt
gaaactaaagattcccaggacagacatgccaatattgaagtttcttacttattacagcgcctggagaattacccgggtctggtcattttatcca
ccaataatcgtggtcatttagacagtgcttttaatcgtcgttttactttcattacccgttttacttacccggatgaaaaaatccgtaaaaaaat
gtggcaggaaatttggcctagaaatataaaaatatcggaagatatcgattttaacgaattagctcaacgaacaagcgtgactggcgcgaatatc
cgcaatattgctttattgtcttcattctttgcttcagagcaggggaatgatgaagtcagtaatgaaaatattgaaattgcattgaagcgtgaat
tagctaaagtcggacgattaacattttaa
SEQ ID NO: 75, pvc15 protein, Species: Photorhabdus asymbiotica
MNISPVFYDSLNQDNDRDLSFLFSELERIDLALQHHFYCVESQRSELLDEFLLTEAEVVT
RLDKPLGKPHWINDDYLAISQKGNVSLMAASRLMDLIERFELTDFERDVLLLGLLPHFD
SRYYRLFSLIQGGQQGRLPSFALALELFCHSALEKQVQQASFLHRAPLMGCQLLSIDTSQ
KTLAWLQTPFITDSGVYHFLLGHHYIMPALEHCAEWLTPTGIGCYPEGLKQVLGNVLLS
DNDNIRPIVLLRGMAGSARAYTITNMMASEGKQTLLVDISKLADSDEKNIILQIKHILRET
RMHGACLLLRNFCLLVEQNKQLLDSLSELLNQPELRIVCLIEPYSPLVWLKKIPVLLIEMP
LLTPAEKARLLIASLPDNCSEDIDTITLSQRYTFNPETLPLILQEAQLYQQQRDPLDILQQC
DIRQALNLRAQQNFGQLAQRIIPKRSLKDLLVSDEIAQQLREILIAIKYREQVLAGGFKDK
IAYGTGISALFYGDSGTGKTMAAEVIADHIGVDLIKVDLSTVVNKYIGETEKNLSRIFDLA
EQDAGVLFFDEADALFGKRSETKDSQDRHANIEVSYLLQRLENYPGLVILSTNNRGHLD
SAFNRRFTFITRFTYPDEKIRKKMWQEIWPRNIKISEDIDFNELAQRTSVTGANIRNIALLS
SFFASEQGNDEVSNENIEIALKRELAKVGRLTF

1. An engineered extracellular contractile injection system (eCIS) comprising:
a structural domain forming a tubular structure;
a targeting domain engineered to target the eCIS to a eukaryotic cell of interest; and
a protein payload,
wherein the targeting domain is capable of targeting the assembled eCIS to the eukaryotic cell of interest.

2. The engineered eCIS of claim 1, wherein the structural domain comprises a tube and a sheath enclosing the tube, a baseplate located at a first end of the tube enclosed by the sheath, and a terminal cap located at a second end of the tube enclosed by the sheath.

3. The engineered eCIS of claim 2,
wherein the tube comprises about 10-40 layers of a tube protein and a plurality of tube connector proteins that connect the tube to the baseplate,
and wherein the sheath comprises about 10-40 layers of a sheath protein and a plurality of sheath connector proteins that connect the sheath to the baseplate,
and wherein the baseplate comprises a plurality of baseplate proteins, and a plurality of spike proteins,
and wherein the terminal cap comprises a terminal cap protein.

4. The engineered eCIS of claim 3, wherein the tube protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 1, or between about 80% to 100% sequence identity to SEQ ID NO: 1, or
wherein the tube protein is encoded by a nucleic acid sequence having at least about 85%, 86%, 87%, 88%, 89%, 90%, 91%, 92%, 93%, 94%, 95% 96%, 97%, 98%, 99% or 100% sequence identity to SEQ ID NO: 2, or between about 80% to 100% sequence identity to SEQ ID NO: 2, and
wherein the plurality of tube connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 3 or 5, or between about 80% to 100% sequence identity to SEQ ID NOs: 3 or 5, or
wherein the plurality of tube connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 4 or 6, or between about 80% to 100% sequence identity to SEQ ID NOs: 4 or 6;
wherein the sheath protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 7, or between about 80% to 100% sequence identity to SEQ ID NO: 7, or wherein the sheath protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 8, or between about 80% to 100% sequence identity to SEQ ID NO: 8;
wherein the plurality of sheath connector proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 9 or 11, or between about 80% to 100% sequence identity to SEQ ID NOs: 9 or 11, or
wherein the plurality of sheath connector proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 10 or 12, or between about 80% to 100% sequence identity to SEQ ID NOs: 10 or 12;
wherein the plurality of baseplate proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 13 or 15, or between about 80% to 100% sequence identity to SEQ ID NOs: 13 or 15, or
wherein the plurality of baseplate proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 14 or 16, or between about 80% to 100% sequence identity to SEQ ID NOs: 14 or 16;
wherein the plurality of spike proteins comprises a sequence having at least about 85%, 86%, 87%, 88%, 89%, 90%, 91%, 92%, 93%, 94%, 95%, 96%, 97%, 98%, 99% or 100% sequence identity to SEQ ID NOs: 19 or 21, or between about 80% to 100% sequence identity to SEQ ID NOs: 19 or 21, or
wherein the plurality of spike proteins is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NOs: 20 or 22, or between about 80% to 100% sequence identity to SEQ ID NOs: 20 or 22; and
wherein the terminal cap protein comprises a sequence having at least about 85%, 86%, 87%, 88%, 89%, 90%, 91%, 92%, 93%, 94%, 95%, 96%, 97%, 98%, 99% or 100% sequence identity to SEQ ID NO:23, or between about 80% to 100% sequence identity to SEQ ID NO:23, or wherein the terminal cap protein is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 24, or between about 80% to 100% sequence identity to SEQ ID NO: 24.

5. The engineered eCIS of claim 1, wherein the targeting domain comprises a tail fiber protein fused to a heterologous binding moiety,
wherein the tail fiber comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 17, or between about 80% to 100% sequence identity to SEQ ID NO: 17, or
wherein the tail fiber is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 18, or between about 80% to 100% sequence identity to SEQ ID NO: 18.

6. The engineered eCIS of claim 5, wherein the heterologous binding moiety is selected from the group consisting of an antibody, an antigen binding fragment, a viral receptor-binding domain, an artificial receptor, a protein tag, and a tail fiber from an orthologous eCIS.

7. The engineered eCIS of claim 6, wherein the antigen binding fragment is selected from the group consisting of a Fab, a Fab′, a F(ab′)₂, an Fd, an Fv, a domain antibody (dAb), a complementarity determining region (CDR), a single chain variable fragment antibody (scFv), a maxibody, a minibody, an intrabody, a diabody, a triabody, a tetrabody, a v-NAR and a bis-scFvs.

8. The engineered eCIS of claim 7, wherein the dAb is a shark antibody or a camelid antibody.

9. The engineered eCIS of claim 1, wherein the eukaryotic cell of interest is a yeast cell, an insect cell, a mammalian cell, a human cell, a plant cell or a fungi cell.

10-11. (canceled)

12. The engineered eCIS of claim 1, wherein the payload comprises an N-terminal packaging domain, wherein the N-terminal packaging domain is between 38 and 67 amino acids long.

13. The engineered eCIS of claim 12, wherein the N terminal packaging domain comprises a sequence having at least about 85%, 86%, 87%, 88%, 89%, 90%, 91%, 92%, 93%, 94%, 95%, 96%, 97%, 98%, 99% or 100% sequence identity to SEQ ID NO: 25, or between about 80% to 100% sequence identity to SEQ ID NO: 25, or
wherein the N terminal packaging domain is encoded by a nucleic acid sequence having at least about 85%, 86%, 87%, 88%, 89%, 90%, 91%, 92%, 93%, 94%, 95%, 96%, 97%, 98%, 99% or 100% sequence identity to SEQ ID NO: 26, or between about 80% to 100% sequence identity to SEQ ID NO: 26.

14. The engineered eCIS of claim 12, wherein the N-terminal packaging domain comprises SEQ ID NO: 26.

15. (canceled)

16. The engineered eCIS of claim 1, wherein the protein payload is a gene editor, a chemotherapy agent, a toxin, a Cre protein, a Cas protein, a Transcription activator-like effector nuclease (TALEN), a Zinc finger nuclease (ZFN), or a ribonucleoprotein.

17-18. (canceled)

19. The engineered eCIS of claim 1, wherein the eCIS is derived from Photorhabdus asymbiotica virulence cassette (PVC), Serratia entomophila anti-feeding prophage (AFP), Pseudoalteromonas luteoviolacea metamorphosis-associated contractile structure (MAC), Amoebophilus asiaticus T6SSiv, Pseudomonas aeruginosa T6SS, or Pseudomonas aeruginosa R-type bacteriocin.

20. The engineered eCIS of claim 1, wherein the eCIS is derived from a Subtype Ia eCIS locus, a Subtype Ib eCIS locus, a Subtype IIa eCIS locus, a Subtype IIb eCIS locus, a Subtype IIc eCIS locus, or a Subtype IId eCIS locus.

21. A method for protein delivery comprising administering an engineered extracellular contractile injection system (eCIS) to a cell, the eCIS comprising:
a structural domain;
a targeting domain engineered to target the eCIS to an eukaryotic cell of interest; and
a protein payload,
wherein the targeting domain is capable of targeting the assembled eCIS to the eukaryotic cell of interest.

22-40. (canceled)

41. A vector system for producing the engineered extracellular contractile injection system (eCIS) of claim 1, wherein the vector system comprises one or more vectors encoding the structural domain and the targeting domain.

42. A host cell comprising or transformed with the vector system of claim 41.

43. A host cell for producing the engineered extracellular contractile injection system (eCIS) of claim 1, wherein the host cell comprising one or more polynucleotides encoding structural domain and the targeting domain.

44. A method for producing the engineered extracellular contractile injection system (eCIS) of claim 1, comprising expressing one or more polynucleotides encoding the structural domain and the targeting domain in a host cell in the presence of the protein payload.
